(19) United States
(12) Patent Application Publication
(10) Pub. No.: US 2026/0124244 A1
(43) Pub. Date: May 7, 2026

(54) MEGAKARYOCYTE DERIVATIVES FOR TREATMENT OF DRY EYE DISEASES

(71) Applicant: Stellular Bio, Inc., Watertown, MA (US)

(72) Inventors: Bradford Dykstra, Brookline, MA (US); Ryan Carpenter, Watertown, MA (US); Sunita Patel-Hett, Watertown, MA (US); Sophia Pete, Watertown, MA (US)

(73) Assignee: Stellular Bio, Inc., Watertown, MA (US)

(21) Appl. No.: 19/258,408

(22) Filed: Jul. 2, 2025

Related U.S. Application Data
(63) Continuation of application No. PCT/US2024/010568, filed on Jan. 5, 2024.
(60) Provisional application No. 63/437,578, filed on Jan. 6, 2023.

(51) Int. Cl. A61K 35/19; A61K 38/18; A61K 38/20; A61P 27/02; C12N 5/078

(52) U.S. Cl. CPC A61K 35/19; A61K 38/1808; A61K 38/2066; A61P 27/02; C12N 5/0644; C12N 2506/45

(57) ABSTRACT

Described herein are methods for treating, repairing or ameliorating diseases, disorders, or injuries related to dry eye disease, wound healing, and osteoarthritis with megakaryocyte derivatives. Also, described herein are methods for generating megakaryocyte derivatives.

### CROSS-REFERENCED APPLICATION

This application is a bypass continuation patent application of PCT International Patent Application No. PCT/US2024/010568, filed Jan. 5, 2024, which claims the benefit of and priority to U.S. Provisional Application No. 63/437,578, filed on Jan. 6, 2023, the entire contents of each of which are incorporated herein by reference.

### BACKGROUND

Blood derivatives, such as platelet rich plasma (PRP), plasma rich in growth factors (PRGF), and autologous serum (AS) have been implicated to play a role in medical treatment of wound healing or speeding repairs of damaged tissues such as cartilage, tendons, ligaments and bone, or in treating dry eye and other diseases. However, there is a paucity of critical data regarding the beneficial effects of these blood derivatives in clinical procedures because of limited sample size of reported data from platelet donors. Moreover, every platelet donor is different, therefore, there is no consistency in the quality of PRP or other derivatives produced for medical procedures. Autologous blood derivatives, currently in use, are plagued with several other disadvantages, such as batch to batch variability because every patient provides his or her own blood as a source of PRP, PRGF, or AS. Other disadvantages include the short life-span of donor platelets, impurities or contaminations, or lack of availability or shortages in supply. As a result of a lack of standardization of these products, clinical effectiveness of blood derivatives is still under debate. Hence, the field is in dire need of a safe, standardized, defined, off-the-shelf product that could be developed and approved for use in humans by regulatory bodies, instead of having to make separate, unreliable preparations for each patient, which has raised skepticism regarding its potential efficacy and use.

Dry eye disease (DED) is an example of a disease for which there is a significant unmet need, and a treatment is urgently desired. In the current DED treatment, patients are prescribed one of a number of treatments, for example Restasis®, which the active ingredient is Cyclosporin A (CsA). Unfortunately, current medications are not effective for many dry eye patients, and their disease often progresses. Dry eye diseases fall into two categories, which are (i) tear-deficient and (ii) evaporative. The evaporative causes of dry eye disease are due to oil deficit, lid changes, use of contact lenses, or ocular surface diseases (OSD), as allergic conjunctivitis, and some of the iatrogenic dry eye that occurs after the use of systemic or topical medications or after surgeries or nonsurgical procedures. If symptoms continue to progress, patients are deemed to require a surgery and are referred to an ophthalmologist. Hence, novel, less aggressive and better therapeutic options for the treatment of dry eye disease are needed and are described in this application.

### SUMMARY

Provided herein are methods of treating, repairing or ameliorating dry eye disease in a subject in need of such treatment. In some embodiments, the methods comprise administering to the subject an effective amount of a composition comprising a megakaryocyte derivative, wherein an administration route is ophthalmic or intraglandular, wherein the megakaryocyte derivative includes megakaryocyte-like cell (MLC) lysates, platelet-like cells (PLC), or combinations thereof.

Provided herein are methods of treating, repairing or ameliorating a condition in a subject in need of such treatment comprising administering to the subject more than one dose of an effective amount of a composition comprising a megakaryocyte derivative, wherein the condition is selected from dry eye disease, ocular surface disease, osteoarthritis, and wound healing, and wherein the megakaryocyte derivative includes MLC lysates, PLCs, exosomes, megakaryocytes, or combinations thereof, and wherein the megakaryocyte derivative comprises one or more biomarkers selected from fibroblast growth factor-2 (FGF-2); hepatocyte growth factor (HGF); insulin-like growth factor 1 (IGF-1), regulated upon activation, normal T cell expressed and secreted (RANTES); nerve growth factor (NGF); vascular endothelial growth factor (VEGF-A); vascular endothelial growth factor (VEGF-C); epidermal growth factor (EGF); transforming growth factor-β1 (TGF-β1); transforming growth factor-β2 (TGF-β2); platelet-derived growth factor-AA (PDGF-AA); platelet-derived growth factor-BB (PDGF-BB); platelet-derived growth factor-AA/BB (PDGF-AA/BB); interleukin 2 (IL-2); interleukin 4 (IL-4); interleukin 12p40 (IL-12p40); interleukin 12p70 (IL-12p70); tissue inhibitors of metalloproteinases-1 (TIMP-1); tissue inhibitors of metalloproteinases-2 (TIMP-2); and tissue inhibitors of metalloproteinases-3 (TIMP-3).

Provided herein are method for producing megakaryocyte derivatives, the methods comprise: an ex-vivo culturing of a population of progenitor cells for a duration to differentiate the progenitor cells to mature megakaryocytes; isolating a population of MLCs, PLCs or derivatives thereof by separating from the megakaryocytes; concentrating the MLCs, PLCs or derivatives thereof; optionally lysing the MLCs, PLCs or derivatives thereof; and optionally mixing with a PRP derived from a donor, wherein the megakaryocyte derivative includes MLC lysates, PLCs, exosomes, megakaryocytes, or combinations thereof.

In some aspects, the techniques described herein relate to a method of treating, repairing or ameliorating a condition in a subject in need of such treatment including administering to the subject an effective amount of a composition including a megakaryocyte derivative, wherein the condition is a dry eye disease, wherein an administration route is ophthalmic or intraglandular, wherein the megakaryocyte derivative includes megakaryocyte-like cell (MLC) lysates, platelet-like cells (PLC), or combinations thereof.

In some aspects, the techniques described herein relate to a method wherein the composition further includes a wound healing agent, a tissue regeneration agent, an antiapoptotic agent, an anti-inflammatory agent, a neurotropic agent, an anti-hormonal agent or an immunomodulatory agent or a combination thereof.

In some aspects, the techniques described herein relate to a method, wherein the tissue regeneration agent is one or more of (a) a growth factor selected from one or more of transforming growth factors (TGF), fibroblast growth factors (FGF), platelet-derived growth factors (PDGF), epidermal growth factors (EGF), vascular endothelial growth factors (VEGF), insulin-like growth factors (IGF), platelet-derived endothelial growth factors (PDEGF), platelet-derived angiogenesis factors (PDAF), platelet factors 4 (PF-4), hepatocyte growth factors (HGF) or combinations thereof; and (b) a cytokine selected from one or more of IL-1B, IL-5, IL-6, IL-8, IL-10, IL-12, IL-13, IL-17A, IL-23, TNF alpha or combinations thereof.

In some aspects, the techniques described herein relate to a method, wherein the megakaryocyte derivative includes one or more biomarkers selected from fibroblast growth factor-2 (FGF-2); hepatocyte growth factor (HGF); insulin-like growth factor 1 (IGF-1), regulated upon activation, normal T cell expressed and secreted (RANTES); nerve growth factor (NGF); vascular endothelial growth factor (VEGF-A); vascular endothelial growth factor (VEGF-C); epidermal growth factor (EGF); transforming growth factor-β1 (TGF-β1); transforming growth factor-β2 (TGF-β1); platelet-derived growth factor-AA (PDGF-AA); platelet-derived growth factor-BB (PDGF-BB); platelet-derived growth factor-AA/BB (PDGF-AA/BB); interleukin 2 (IL-2); interleukin 4 (IL-4); interleukin 12p40 (IL-12p40); interleukin 12p70 (IL-12p70); tissue inhibitors of metalloproteinases-1 (TIMP-1); tissue inhibitors of metalloproteinases-2 (TIMP-2); and tissue inhibitors of metalloproteinases-3 (TIMP-3).

In some aspects, the techniques described herein relate to a method, wherein the megakaryocyte derivative includes EGF concentration per unit total protein that is higher than an average EGF concentration per unit total protein measured in human platelets, platelet-rich plasma (PRP), or plasma rich in growth factors (PRGF).

In some aspects, the techniques described herein relate to a method, wherein the megakaryocyte derivative is produced from cells derived from reprogramming and/or differentiation of a somatic cell, progenitor cell or stem cell.

In some aspects, the techniques described herein relate to a method, wherein the megakaryocyte derivative is not a cancerous cell.

In some aspects, the techniques described herein relate to a method, wherein the megakaryocyte derivative does not exhibit uncontrolled growth or tumor formation in vivo.

In some aspects, the techniques described herein relate to a method, wherein the method reduces injury recovery time.

In some aspects, the techniques described herein relate to a method, wherein the method improves one or more signs and/or symptoms of dry eye disease.

In some aspects, the techniques described herein relate to a method, wherein the composition further includes lysates, serum, plasma, plasma rich in growth factors (PRGF), platelet rich plasma (PRP), or any other blood derivative derived from the subject.

In some aspects, the techniques described herein relate to a method, wherein the ophthalmic administration route is subconjunctival or topical.

In some aspects, the techniques described herein relate to a method, wherein the ophthalmic administration route is sub-Tenon's.

In some aspects, the techniques described herein relate to a method, wherein the ophthalmic administration route is intravitreal or intracameral.

In some aspects, the techniques described herein relate to a method, wherein the dry eye disease is caused by Sjogren's syndrome or non-Sjogren's syndrome.

In some aspects, the techniques described herein relate to a method, wherein the megakaryocyte derivative is free of red blood cells or hemoglobin content or white blood cells.

In some aspects, the techniques described herein relate to a method, wherein the composition further includes extracellular vesicles (EV).

In some aspects, the techniques described herein relate to a method, wherein the composition is formulated for application to a site of injury or tissue damage for therapeutic use.

In some aspects, the techniques described herein relate to a method, wherein the composition in formulated in a buffer, diluent, or excipient or a combination thereof.

In some aspects, the techniques described herein relate to a method, wherein the composition includes: a) between 0.01 and 100 wt % of the megakaryocyte derivative, b) between 0 and 90 wt. % of a bulking agent, and/or c) between 0 and 90 wt. % of at least one excipient or carrier and optionally d) platelet-rich plasma (PRP), PRGF, plasma, serum, or other blood derivative derived from the subject.

In some aspects, the techniques described herein relate to a method, wherein the composition is lyophilized.

In some aspects, the techniques described herein relate to a method, wherein the composition is implanted on an implantable device.

In some aspects, the techniques described herein relate to a method, wherein the composition is cryopreserved.

In some aspects, the techniques described herein relate to a method, wherein the composition is locally administered at one or more of a site of or near an injury or a disease.

In some aspects, the techniques described herein relate to a method, wherein the composition further includes another therapeutic agent.

In some aspects, the techniques described herein relate to a method of treating, repairing or ameliorating a condition in a subject in need of such treatment including administering to the subject more than one dose of an effective amount of a composition including a megakaryocyte derivative, wherein the condition is selected from dry eye disease, ocular surface disease, osteoarthritis, and wound healing, wherein the megakaryocyte derivative includes MLC lysates, PLCs, exosomes, megakaryocytes, or combinations thereof, and wherein the megakaryocyte derivative includes one or more biomarkers selected from fibroblast growth factor-2 (FGF-2); hepatocyte growth factor (HGF); insulin-like growth factor 1 (IGF-1), regulated upon activation, normal T cell expressed and secreted (RANTES); nerve growth factor (NGF); vascular endothelial growth factor (VEGF-A); vascular endothelial growth factor (VEGF-C); epidermal growth factor (EGF); transforming growth factor-β1 (TGF-β1); transforming growth factor-β2 (TGF-β1); platelet-derived growth factor-AA (PDGF-AA); platelet-derived growth factor-BB (PDGF-BB); platelet-derived growth factor-AA/BB (PDGF-AA/BB); interleukin 2 (IL-2); interleukin 4 (IL-4); interleukin 12p40 (IL-12p40); interleukin 12p70 (IL-12p70); tissue inhibitors of metalloproteinases-1 (TIMP-1); tissue inhibitors of metalloproteinases-2 (TIMP-2); and tissue inhibitors of metalloproteinases-3 (TIMP-3).

In some aspects, the techniques described herein relate to a method, wherein the composition further includes a wound healing agent, a tissue regeneration agent, an antiapoptotic agent, an anti-inflammatory agent, a neurotropic agent, an anti-hormonal agent or an immunomodulatory agent or a combination thereof.

In some aspects, the techniques described herein relate to a method, wherein the more than one dose is administered, daily, weekly, biweekly, triweekly or monthly.

In some aspects, the techniques described herein relate to any one of the methods provided herein, wherein the composition is administered by any one of route of administrations selected from topical, transdermal, or systemic route of administration.

In some aspects, the techniques described herein relate to any one of the methods provided herein, wherein the composition is administered by any one of route of administrations selected from subconjunctival, sub-Tenon's, intravitreal, intracameral, intravenous, intraarterial, intramuscular, subcutaneous, inhalation, rectal, buccal, vaginal, intraperitoneal, intraarticular, intraglandular into a lacrimal gland, topical, otic, or oral route of administration.

In some aspects, the techniques described herein relate to a method, wherein: the conditions is a dry eye disease, the treatment is not for cancer, and an administration route is ophthalmic, intraglandular, subconjunctival, topical, sub-Tenon's, intravitreal, or intracameral.

In some aspects, the techniques described herein relate to a method, wherein the composition is diluted to a physiological concentration in a carrier, wherein the carrier includes a diluent or an excipient.

In some aspects, the techniques described herein relate to a method, wherein the carrier is a plasma or a plasma substitute or a plasmalyte or saline.

In some aspects, the techniques described herein relate to a method for producing a megakaryocyte derivative, the method including: an ex-vivo culturing of a population of progenitor cells for a duration to differentiate the progenitor cells to mature megakaryocytes; isolating a population of MLCs, PLCs or derivatives thereof by separating from the megakaryocytes; concentrating the MLCs, PLCs or derivatives thereof; optionally lysing the MLCs, PLCs or derivatives thereof; and optionally mixing with a PRP derived from a donor, wherein the megakaryocyte derivative includes MLC lysates, PLCs, exosomes, megakaryocytes, or combinations thereof.

In some aspects, the techniques described herein relate to a method, wherein the progenitor cells are selected from one or more of human induced pluripotent stem cells (iPSCs), hematopoietic stem cells, embryonic stem cells (ESCs), immortalized megakaryocytic progenitor cells, CD34+ umbilical cord blood stem cells (UCB cells), CD34+ mobilized peripheral blood cells (MPB cells) or CD34+ bone marrow cells.

In some aspects, the techniques described herein relate to a composition for treating a dry eye disease in a subject, the composition made according to any one of the methods provided herein.

In some aspects, the techniques described herein relate to a composition, wherein the megakaryocyte derivative includes one or more biomarkers selected from fibroblast growth factor-2 (FGF-2); hepatocyte growth factor (HGF); insulin-like growth factor 1 (IGF-1), regulated upon activation, normal T cell expressed and secreted (RANTES); nerve growth factor (NGF); vascular endothelial growth factor (VEGF-A); vascular endothelial growth factor (VEGF-C); epidermal growth factor (EGF); transforming growth factor-β1 (TGF-β1); transforming growth factor-β2 (TGF-β1); platelet-derived growth factor-AA (PDGF-AA); platelet-derived growth factor-BB (PDGF-BB); platelet-derived growth factor-AA/BB (PDGF-AA/BB); interleukin 2 (IL-2); interleukin 4 (IL-4); interleukin 12p40 (IL-12p40); interleukin 12p70 (IL-12p70); tissue inhibitors of metalloproteinases-1 (TIMP-1); tissue inhibitors of metalloproteinases-2 (TIMP-2); and tissue inhibitors of metalloproteinases-3 (TIMP-3).

In some aspects, the techniques described herein relate to a composition, wherein the megakaryocyte derivative includes EGF concentration per unit total protein that is higher than an average EGF concentration per unit total protein measured in human platelets, platelet-rich plasma (PRP), or plasma rich in growth factors (PRGF).

In some aspects, the techniques described herein relate to a composition, wherein the composition further includes a wound healing agent, a tissue regeneration agent, an antiapoptotic agent, an anti-inflammatory agent, a neurotropic agent, an anti-hormonal agent or an immunomodulatory agent or a combination thereof.

In some aspects, the techniques described herein relate to a composition, wherein the megakaryocyte derivative is free of red blood cells or hemoglobin content or white blood cells.

In some aspects, the techniques described herein relate to a composition, wherein the composition further includes extracellular vesicles (EV).

In some aspects, the techniques described herein relate to a composition, wherein the composition is formulated for application to a site of injury or tissue damage for therapeutic use.

In some aspects, the techniques described herein relate to a composition, wherein the composition further includes another therapeutic agent.

In some aspects, the techniques described herein relate to any one of the methods provided herein, wherein the megakaryocyte derivative is made in a fluidic device or a bioreactor.

### BRIEF DESCRIPTION OF FIGURES

The present disclosure will be described in the detailed description which follows, in reference to the noted plurality of drawings by way of non-limiting examples of exemplary embodiments, in which like reference numerals represent similar parts throughout the several views of the drawings, and wherein:

FIGS. 1A-1C show growth factor and cytokine profile in PLCs as compared to donor PRP and washed donor platelets.

FIG. 2A shows a schematic design of a PLC treatment study for dry eye disease in mice. FIGS. 2B-2C show corneal fluorescein staining and phenol red thread tests results, respectively, to evaluate dry eye disease-induced mice when treated with PLCs as compared to Cyclosporin A (CsA) as a positive control. FIG. 2D illustrates PLC-mediated amelioration of goblet cell loss in the DS-induced DED model, as measured by differences in goblet cell density in the conjunctiva of vehicle-treated versus PLC-treated mice. FIG. 2E shows representative histological images of PAS stained conjunctiva of a vehicle-treated versus a PLC-treated mouse. Goblet cells stain dark along the margins of the conjunctiva.

FIG. 3 illustrates combined corneal fluorescein staining data from the study presented in FIG. 2 and a follow-up study in the same DED model.

FIG. 4A shows a schematic design of a PLC treatment study for Sjogren's Syndrome using in vivo TSP1 knockout mouse model. FIG. 4B summarizes the results for each group pooled from several studies as measured by the average change in corneal fluorescein score for each eye, by comparing each eye after 2 weeks of treatment compared to the same eye before treatment, at baseline (i.e., before treatment) and at endpoint (i.e., after treatment). FIG. 4C illustrates representative images with marked areas of corneal fluorescein staining. FIG. 4D illustrates further time course results from four arms of the study, in which the same eyes were again measured 2 weeks after cessation of treatment.

FIGS. 5A-5B are heat map representations of profiled cytokines and growth factors, illustrating a relative concentration of the cytokines and growth factors as measured by the profiling in MLC and a variety of MLC derivatives, compared to human blood derived PRGF, and in in PLC and a variety of MLC derivatives, compared to PRGF derived from human blood, respectively.

FIGS. 6A-6D provides comparative analysis of the measured concentrations of key cytokines and other factors in MLC and a variety of MLC derivatives, compared to PRGF derived from human blood. FIG. 6A shows Key Platelet/Plasma Associated Factors. FIG. 6B shows putative Corneal Epitheliotrophic factors. FIG. 6C shows putative Pro-Resolutory T-cell factors. FIG. 6D shows Matrix Metalloproteinase Inhibitors.

FIGS. 7A-7D provides comparative analysis of the measured concentrations of key cytokines and other factors in MLC and a variety of MLC derivatives, compared to PRGF derived from human blood. FIG. 7A shows Key Platelet/Plasma Associated Factors. FIG. 7B shows putative Corneal Epitheliotrophic factors. FIG. 7C shows putative Pro-Resolutory T-cell factors. FIG. 7D shows Matrix Metalloproteinase Inhibitors.

FIG. 8A illustrates a total protein analysis (BCA). FIGS. 8B and 8C provide ELISA-based quantification of EGF and PDGF-BB in various MLC derivatives, respectively.

FIGS. 9A-9B provide a particle distribution and particle concentration (1750-10000 nm) in various megakaryocyte derivative preparations.

FIG. 10A shows results of effects of EGF on proliferation of HCE-T cells in a 72-hour time course study at doses ranging from 30.4 pg/ml to 31.2 ng/ml. FIG. 10B quantifies the data from 10A using area under the curve normalized to vehicle.

FIG. 11A shows results of effects of MLC lysate on proliferation of HCE-T cells in a 72-hour time course study at a dose ranging from 0.025% (v/v) to 0.75% (v/v), and FIG. 11B quantifies the data from 11A using area under the curve normalized to vehicle. FIG. 11C shows results of effects of MLC lysate prepared by 0.22 μm filtration on proliferation of HCE-T cells in a 72-hour time course study at doses ranging from 0.0128% (v/v) to 1.0% (v/v). FIG. 11D quantifies the data from 11C using area under the curve normalized to vehicle.

FIGS. 12A-12B illustrate results of carboxyfluorescein diacetate succinimidyl ester (CFSE)-based flow cytometry assay. FIG. 12A is raw MFI (mean fluorescence intensity), with lower MFI indicating higher proliferative index. FIG. 12B is the same data shown as a normalized ratio, with higher ratio indicating higher proliferative index.

FIG. 13A shows a schematic design of a study to evaluate in vivo effects of megakaryocyte derivative treatment in rat medial meniscal tear (MMT) model of osteoarthritis (OA). FIGS. 13B-13G illustrate efficacy results of megakaryocyte derivatives, MLC lysate and PLC, on dynamic weight bearing test (FIG. 13B), electronic von frey filament analysis (FIG. 13C), histology of MMT joint (FIG. 13D), substantial cartilage degeneration width (FIG. 13E), synovitis score (FIG. 13F), and medial tibial osteophyte measurement results (FIG. 13G).

FIGS. 14A-14B illustrate the effect of PLCs on HUVEC migration in scratch assays. FIG. 14A illustrates results of the time course data (120 hours) of the resulting cell index, and FIG. 14B illustrates AUC data normalized to the vehicle.

FIG. 15A shows a schematic design of a study for evaluation in vivo effects of megakaryocyte derivatives, MLC lysate and PLC, treatment in a db/db diabetic mouse model of wound healing. FIGS. 15B-15F illustrate efficacy results of PLC and MLC lysate, including wound closure kinetics (FIG. 15B), histology results of diabetic mouse wounds (FIG. 15C), histological analysis of wounds for the quantification of wound closure (FIG. 15D), granulation tissue area (FIG. 15E) and epidermis tissue area (FIG. 15F), endomucin-based histological visualization of vasculature (FIG. 15G), and quantification of the endomucin fluorescent signal (FIG. 15H).

FIG. 16 illustrates a schematic of the differentiation process.

FIG. 17A illustrates a schematic of the PLC production process. FIG. 17B illustrates an exemplary microfluidic bioreactor (BioR) device for processing the MLCs.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R Rieger et al. (eds.), Springer Verlag (1991); and Hale & Marham, The Harper Collins Dictionary of Biology (1991). As used herein, the following terms have the meanings ascribed to them below, unless specified otherwise.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, phosphate buffered saline (PBS), citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; plasma or plasma substitutes, dextran and hydroxyethyl starch, perfluorocarbons and stroma-free hemoglobin; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

The term “derivatives”, as used herein, refer to composition produced from cells, either by differentiation into different types of cells, genetically engineering or genetically modifying such cells, or compositions produced from such cells (for example, by lysing such cells). In other words, the term derivative is inclusive of any modification, genetic, chemical or a combination thereof otherwise of the cells.

The term “megakaryocyte derivatives,” as used herein, includes megakaryocytes or MLC derivatives. In some embodiments, megakaryocyte derivatives include any composition produced from megakaryocytes or MLCs, (for example, lysates, EVs) or other cells differentiated from megakaryocytes or MLCs. In some embodiments, megakaryocyte derivatives include MLCs, PLCs, derivatives thereof or lysates thereof or PRP, PRGF, or AS derived therefrom. In some embodiments, megakaryocyte derivatives include compositions produced from genetically engineered MLCs, (eMLCs) or PLCs (ePLCs).

As used herein “progenitor cells” refers to IPSC-derived cells, such as preMKs, MKs, proplatelets, preplatelets. It is also inclusive of “pluripotent stem cells”, which includes embryonic stem cells, embryo-derived stem cells, and induced pluripotent stem cells and other stem cells having the capacity to form cells from all three germ layers of the body, regardless of the method by which the pluripotent stem cells are derived. Pluripotent stem cells are defined functionally as stem cells that can have one or more of the following characteristics: (a) be capable of inducing teratomas when transplanted in immunodeficient (SCID) rats; (b) capable of differentiating to cell types of all three germ layers (e.g., can differentiate to ectodermal, mesodermal, and endodermal cell types); or (c) express one or more markers of embryonic stem cells (e.g., express Oct 4, alkaline phosphatase, SSEA-3 surface antigen, SSEA-4 surface antigen, SSEA-5 surface antigen, Nanog, TRA-1-60, TRA-1-81, SOX2, or REX1). Progenitor cells also include “megakaryocytic progenitor” (preMK), which refers to a mononuclear hematopoietic cell that is committed to the megakaryocyte lineage and is a precursor to mature megakaryocytes. Megakaryocytic progenitors are normally found in (but not limited to) bone marrow and other hematopoietic locations, but can also be generated from pluripotent stem cells, such as by further differentiation of hemogenic endothelial cells that were themselves derived from pluripotent stem cells.

The term “megakaryocytic progenitor” (preMK), as used herein, refers to a mononuclear hematopoietic cell that is committed to the megakaryocyte lineage and is a precursor to mature megakaryocytes. Megakaryocytic progenitors are normally found in (but not limited to) bone marrow and other hematopoietic locations, but can also be generated from pluripotent stem cells, such as by further differentiation of hemogenic endothelial cells that were themselves derived from pluripotent stem cells.

The term “induced pluripotent stem cells” (iPS cells or iPSCs) refers to a type of pluripotent stem cell generated by reprogramming a somatic cell by expressing a combination of reprogramming factors. The iPSCs can be generated using fetal, postnatal, newborn, juvenile, or adult somatic cells. Factors that can be used to reprogram somatic cells to pluripotent stem cells include, for example, a combination of Oct 4 (sometimes referred to as Oct 3/4), Sox2, c-Myc, and Klf4. In some embodiments, factors that can be used to reprogram somatic cells to pluripotent stem cells include, for example, a combination of Oct 4, Sox2, Nanog, and Lin28. In some embodiments, at least two, three, or four reprogramming factors are expressed in a somatic cell to reprogram the somatic cell.

“Agonist Activated” cell receptor or ligand activation induced by a receptor specific agonist. Agonists activate cells by binding to their respective receptors or ligands on a cell.

“Variant” as used herein refers to manifesting structural variety, structural deviation, or structural differences. Variants also include PLCs or derivatives thereof or the PLCs or the microsome, exosomes, vesicles or any other product generated by culturing the megakaryocytes in a bioreactor.

“PLC” or “PLCs” or artificial platelets as interchangeably used herein, refer to non-naturally existing, novel, anucleated platelets or platelet-like cells that structurally differ from naturally existing bone marrow derived platelets (i.e., natural counterpart). PLC or PLCs are also inclusive of platelet variants, defined elsewhere.

“MLC” or “MLCs” or artificial megakaryocytes as interchangeably used herein, refer to non-naturally existing, novel, megakaryocytes or megakaryocytes-like cells that structurally differ from naturally existing bone marrow derived megakaryocytes (i.e., natural counterpart).

As used herein, “Variant” or “Variants” as interchangeably used herein refers to manifesting structural variety, structural deviation, or structural differences between PLCs and donor platelets. As non-limited examples, variant comprises greater than an average of 2% CD63 receptors (i.e., CD63^{>average2%}) as compared to the reference resting bone marrow derived platelet cells with less than an average 2% CD63 receptor i.e., (CD63^{<average 2%}). In some embodiments, a variant comprises less than 10% on an average of CD36 receptor (i.e., CD36^{<average80%}) as compared to the reference resting bone marrow derived platelet cells with greater than an average 80% CD36 receptor i.e., (CD36^{>average 80%}); or a variant comprising less than an average of 95% CD42b receptor (i.e., CD42^{<average95%}) as compared to the reference resting bone marrow derived platelet cells with greater than an average 95% CD42b receptor i.e., (CD42b^{>average 95%}); or a variant comprising less than an average of 90% glycoprotein VI receptor or less i.e., (GPVI^{<average90%}) as compared to the reference resting bone marrow derived platelet cells with greater than an average 90% GPVI receptor i.e., (GPVI^{>average 90%}). The term variant is also inclusive of a structural makeup of the PLCs that is comparable to the structural make-up of naturally existing bone marrow derived platelets, either in resting or in their activated stages. For example, the PLCs and the donor platelets may have m % CD36, or n % CD42a, or o % CD42a-b-d, or p % CD61, or q % CD62p, or x % CD63 receptors, where the m %, n %, o %, p %, q % x % are the same (i.e., have equal values) between the PLCs and the and bone marrow derived platelets. In other words, structurally PLCs may be identical to donor platelets, yet manifest the advantages of the PLC variants disclosed herein in the present disclosure.

By, “comprises,” “comprising,” “containing” and “having” and the like can have the meaning ascribed to them in U.S. patent law and can mean “includes,” “including,” and the like (.g., a composition “comprising” X may consist exclusively of X or may include something additional, e.g., X+Y); “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

“Average” as used herein is a number expressing the central or typical value in a set of data, in particular the mode, median, or (most commonly) the mean, which is calculated by dividing the sum of the values in the set by their number. It also refers to a single value (such as a mean, mode, or median) that summarizes or represents the general significance of a set of unequal values.

“Non-natural” as used herein refers to manufactured, created, or constructed by human beings, artificial, or mimicking something that exists in nature.

Routes of administration for the various embodiments include, but are not limited to, local at a site in need of treatment (e.g., a surface of an eye in dry eye treatment, a knee in osteoarthritis), intraglandular, topical, transdermal, nasal, systemic administration (such as, intravenous, intraarterial, intramuscular, subcutaneous, inhalation, rectal, buccal, vaginal, intraperitoneal, intraarticular, ophthalmic, otic, or oral administration), and ophthalmic administration (such as subconjunctival, topical, sub-Tenon's, intravitreal, or intracameral). As used herein, “systemic administration” refers to all nondermal routes of administration, and specifically excludes topical and transdermal routes of administration. Additional ways for administering MLCs, PLCs or derivatives thereof of the present disclosure include, intrathecal, intracapsular, intraorbital, intracardiac, intradermal, intraperitoneal, transtracheal, subcutaneous, subcuticular, intraarticular, subcapsular, subarachnoid, intraspinal and intersternal injection and infusion.

The term “antagonist” as used herein refers to a substance which initiates a physiological response when combined with a receptor. Agonists activate cells by binding to their respective receptors or ligands on a cell. It can be any agent or entity capable of inhibiting the expression or activity of a protein, polypeptide portion thereof, or polynucleotide. Thus, the antagonist may operate to prevent transcription, translation, post-transcriptional or post-translational processing or otherwise inhibit the activity of the protein, polypeptide, or polynucleotide in any way, via either direct or indirect action. The antagonist may for example be a nucleic acid, peptide, or any other suitable chemical compound or molecule or any combination of these. Additionally, it will be understood that in indirectly impairing the activity of a protein, polypeptide of polynucleotide, the antagonist may affect the activity of the cellular molecules which may in turn act as regulators or the protein, polypeptide, or polynucleotide itself. Similarly, the antagonist may affect the activity of molecules which are themselves subject to the regulation or modulation by the protein, polypeptide of polynucleotide. The term “Agonist Activated”, as used herein, refers to cell receptor or ligand activation induced by a receptor specific agonist.

“Donor platelets” refer to bone marrow derived platelets physiologically generated in a mammalian (e.g., human) body. Donor PRP refers to PRP prepared from a donor's blood.

The term “subject’ as used herein refers to a mammal, such as but not limited to human, porcine, equine, dogs or cats or any other animal capable of suffering from viral infection. It is inclusive of a healthy population, a population potentially infected with a pathogen or a patient suffering from a viral infection.

The term “drug”, “agent” or “compound” as used herein refers to a biological product or a chemical entity or combination of biological product and chemical entities, administered in a therapeutic amount to a person to treat or prevent or control a disease or condition. The biological product or the chemical entity is inclusive of but not limited to, antibodies or fragments thereof, a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “agent” “therapeutic composition,” or “therapeutic agent” can be used interchangeably and refers to a therapeutic agent. Agent may be selected from one or more of proteins; peptides; aptamers; antibodies; or fragments thereof; chemicals; small molecules; nucleic acid sequences; nucleic acid analogues. A nucleic acid sequence may be RNA or DNA, and may be single or double stranded, and can be selected from; nucleic acid encoding a protein of interest, oligonucleotides, nucleic acid analogues, for example peptide-nucleic acid (PNA), pseudo-complementary PNA (pc-PNA), locked nucleic acid (LNA), etc. Such nucleic acid sequences include, for example, but not limited to, nucleic acid sequence encoding proteins, for example that act as transcriptional repressors, antisense molecules, ribozymes, small inhibitory nucleic acid sequences, for example but not limited to RNAi, shRNAi, siRNA, micro-RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins, and fragments thereof. The agent may be applied to the media, where it contacts the cell and induces its effects. Alternatively, the agent may be intracellular within the cell because of introduction of the nucleic acid sequence into the cell and its transcription resulting in the production of the nucleic acid and/or protein environmental stimuli within the cell. In some embodiments, the agent is any chemical, entity or moiety, including without limitation synthetic and naturally occurring non-proteinaceous entities. In some embodiments the agent is a small molecule having a chemical moiety. For example, chemical moieties included unsubstituted or substituted alkyl, aromatic, or heterocyclyl moieties including macrolides, leptomycin and related natural products or analogues thereof. Agents can be known to have a desired activity and/or property or can be selected from a library of diverse compounds.

The term “antibody,” as used herein, refers to an immunoglobulin molecule which specifically binds with an antigen. The term “antibody fragment” refers to a portion of an intact antibody and refers to the antigenic determining variable regions of an intact antibody.

The term “culture conditions” or a “culture medium” or “medium” can be used interchangeably and refers to a medium for culturing cells containing nutrients that maintain cell viability and support cell expansion and maintenance stage or the cell differentiation stage. The cell culture medium, in addition to the embodiments disclosed herein, may contain any of the following in an appropriate combination: salt(s), buffer(s), amino acids, glucose or other sugar(s), antibiotics, serum or serum replacement, and other components such as peptide growth factors, etc. The appropriate cell culture media, for a cell type, is known to those skilled in the art.

As used herein, the terms “treat,” “treating,” “treatment,” and the like refer to reducing or ameliorating a disorder and/or symptom associated therewith. It will be appreciated that, although not precluded, treating a disorder or condition does not require that the disorder, condition, or symptoms associated therewith be eliminated.

The term “PLC-enriched plasma” as used herein refers to the enriched PRP or enriched plasma provided by a combination of intact PLCs or derivatives thereof or precursor cells for making the PLCs or derivatives, lysates extracted therefrom to enrich other source of plasma sources, such as a donor-derived PRP. Optionally, it could include growth factors, cytokines or other agents from other sources, which complement the therapeutic applications of the PLCs or derivatives thereof.

Specifically, stated, or obvious from context, as used herein, the term “or” is understood to be inclusive. Unless specifically stated or obvious from context, as used herein, the terms “a”, “an”, and “the” are understood to be singular or plural.

The word “substantially” does not exclude “completely” e.g., a composition which is “substantially free” from Y may be completely free from Y. Where necessary, the word “substantially” may be omitted from the definition of the disclosure.

The term “extracellular vesicles (EVs or EV)” as used herein collectively refers to microvesicles and exosomes and generally are very small (generally around 1 micron or less in diameter; microvesicles, generally about 200-1500 nm or less in diameter; exosomes generally about 20-200 nm or less in diameter) phospholipid vesicle shed from a megakaryocyte or other cell. Extracellular vesicles (EVs) may contain or may transport materials such as but not limited to nucleic acids (e.g., siRNA), growth factors, proteins or exogenous genetic materials (e.g., for gene therapy) and express the extracellular markers of their parental cells. Megakaryocyte-derived extracellular vesicles (EVs) may have a role in multiple pathways, including hemostasis and inflammation, and in treating various disorders, such as but not limited to, malignancies (e.g., neoplasia), Alzheimer, and tumor progression and development.

The term “cryopreservation medium” refers to a liquid medium (solution or suspension), capable of preserving structure and metabolism of isolated cells against injury associated with freezing events either within or exterior to the cells and that is safe for infusion or injection into humans. The term further refers to a medium (solution or suspension) containing components, including cryopreservation agents, also determined or known to be safe for human infusion or injection. Preferably, the medium (solution or suspension), and agents, components or elements of the medium are approved by a United States regulatory agency for infusion or injection into humans, e.g., histidine (25-50 mM). “Cryoprotective agents” are agents that are capable of conferring a degree of cryoprotection to cell structure and metabolism upon freezing. Cryoprotective agents within the scope of the present disclosure include arabinogalactan and biological and functional equivalents thereof, glycerol, propylene glycol, and albumin, e.g., human serum albumin, plasma or serum.

The term “therapy” is intended to encompass any form of treatment, prevention, or diagnosis, and includes treatments to both cure and prevent disease. Thus, treatment of a healthy patient is to be considered as therapy. Therapy also covers the alleviation of symptoms, in addition to curative treatments for a disease.

The compositions and methods of the present disclosure take advantage of composition of megakaryocyte derivatives. The composition comprises growth factors, cytokines and other megakaryocyte-derived components, proteinaceous or otherwise, offering a unique advantage to maximize therapeutic outcomes as well as minimizing side effects in treating, repairing or ameliorating diseases, disorders, or injuries related to dry eye, osteoarthritis, tendon, ligament, bone repair, wound healing or wound-healing related disorders, alopecia or skin rejuvenation or regeneration for which no adequate or consistent treatments are available by convention means. In some embodiments, the megakaryocyte derivatives comprise megakaryocyte-like cells (MLCs) (including that of engineered MLCs). In some embodiments, the megakaryocyte derivatives comprise MLC lysate. In some embodiments, the megakaryocyte derivatives comprise PLCs or derivative thereof (including that of engineered PLCs).

In some embodiments, the megakaryocyte derivatives are derived from reprogramming of a somatic cell, progenitor cell or stem cell, products of which passage through a bioreactor. In some embodiments, the megakaryocyte derivatives do not comprise a cancerous cell. In some embodiments, the megakaryocyte derivatives do not exhibit uncontrolled growth or tumor formation in vivo.

In some embodiments, the megakaryocyte derivatives ameliorate goblet cell loss in dry eye disease. In some embodiments, the megakaryocyte derivatives are used for treating or ameliorating symptoms of Sjogren's Syndrome. In some embodiments, topical treatment of an eye of a subject with the megakaryocyte derivatives improve corneal barrier integrity relative to an eye of a subject treated with vehicle. In some embodiments, the megakaryocyte derivatives comprise high concentrations of epitheliotrophic and platelet associated factors, TIMPS that inhibit MMP tissue degradation, Interleukins or growth factors that polarize T-cells to secrete pro-resolutory cytokines, or combinations thereof compared to PRGF from human blood. In some embodiments, the megakaryocyte derivatives comprise high concentrations of epidermal growth factor (EGF) compared to PRGF from human blood. In some embodiments, the megakaryocyte derivatives promote corneal epithelial cell proliferation. In some embodiments, the growth factors are able to stimulate corneal epithelial cell migration and growth. In some embodiments, the megakaryocyte derivatives contain immune modulators which mitigate immune cell-mediated cell loss. In some embodiments, the megakaryocyte derivatives contain neurotrophic factors which protect corneal neural cells and/or stimulate corneal neural cell regeneration. In some embodiments, treatment with the megakaryocyte derivatives helps preserving intact articular cartilage and/or improves/reverses cartilage degeneration condition in osteoarthritis subjects in a subject having osteoarthritis relative a subject treated with vehicle only. In some embodiments, treatment with the megakaryocyte derivatives reduces rate of cartilage degeneration or reverses cartilage degeneration in a subject having osteoarthritis relative a subject treated with vehicle only. In some embodiments, treatment with the megakaryocyte derivatives promotes wound healing in a subject relative a subject treated with vehicle only. In some embodiments, treatment with the megakaryocyte derivatives promotes wound healing in a diabetic subject relative a diabetic subject treated with vehicle only.

The compositions and methods of the present disclosure take advantage of properties of PLCs or derivatives thereof (including that of engineered PLCs) such as but not limited to providing growth factors, cytokines and other agents to offer a unique opportunity to maximize therapeutic outcomes as well as minimizing side effects in treating, repairing or ameliorating diseases, disorders, or injuries related to dry eye, osteoarthritis, tendon, ligament, bone repair, wound healing or wound-healing related disorders, alopecia or skin rejuvenation or regeneration for which no adequate or consistent treatments are available by convention means.

Prior art protocol for platelet rich plasma (PRP) enrichment for its use is essentially performed in multiples steps thereby increasing the risk of contamination and leading to inconsistencies an unreliability of the product. For example, typical preparation off PRP includes multiple steps: (1) a small amount of venous blood (15-50 mL) is drawn from the patient's arm in anticoagulant-containing tubes; (2) the recommended temperature during processing is 21° C.-24° C. to prevent platelet activation during centrifugation of the blood; (3) the blood is centrifuged at 1,200 rpm for 12 minutes; (4) the blood separates into three layers: an upper layer that contains platelets and white blood cells, an intermediate thin layer (the buffy coat) that is rich in white blood cells, and a bottom layer that contains red blood cells; (5) the upper and intermediate buffy layers are transferred to an empty sterile tube. The plasma is centrifuged again at 3,300 rpm for 7 minutes to help with the formation of soft pellets (erythrocytes and platelets) at the bottom of the tube; (6) the upper two-thirds of the plasma is discarded because it is platelet-poor plasma; (7) pellets are homogenized in the lower third (5 mL) of the plasma to create the PRP ready for injection.

The present disclosure eliminates many of the prior art steps involved in PRP preparation and serum preparation thereby minimizing risk of contamination or impurities. Most importantly, it eliminates the need to draw venous blood, often a source of contamination (e.g., viruses, such as HIV) unless analyzed critically. In other words, the present disclosure, at the very least, eliminates the need of drawing blood or the separation of blood into different components before platelets can isolated. This is accomplished by culturing a population of progenitor stem cells until conditions such that they substantially differentiate into matured megakaryocytes. Next, the matured megakaryocytes are cultured in a bioreactor or a fluidic device, wherein a bioreactor gradient in the bioreactor mimics endogenous platelet producing environment to generate PLCs (PLCs) or derivatives thereof. The MLCs, PLCs or derivatives thereof are then collected from the bioreactor in an amount sufficient to use the MLCs, the PLCs or their derivatives. In some embodiments, the MLCs, the PLCs or their derivatives or lysates thereof can be used on their own or in combination with the EVs. In some embodiments, MLCs, PLCs or derivatives thereof or lysates thereof can be used to enrich the donor platelets in a platelet rich plasma (i.e., PLCs or genetically engineered PLCs are mixed with donor-based PRP). In some embodiments, MLCs, PLCs or derivatives thereof can be used in combination with the EVS or with other drugs disclosed herein.

The megakaryocyte derivatives (e.g., MLCs, PLCs or derivatives thereof or lysates thereof or PRP, PRGF, or AS derived therefrom) or precursor cells for making megakaryocyte derivatives (MLCs, PLCs or derivatives thereof or lysates thereof or PRP, plasma rich in growth factors (PRGF), or autologous serum (AS) derived therefrom), which are rich in growth factor and cytokines, can be used in their intact form or in the form of a liquid, a paste, or can be admixed with other agents, such as but not limited to gel, ointment, creams or other emulsifying agents, acceptable diluent, carrier or excipient.

In some embodiments, the present disclosure further provides pharmaceutical composition comprising megakaryocyte derivatives (e.g., MLCs, PLCs or derivatives thereof or lysates thereof or PRP, PRGF, or AS derived therefrom) or precursor cells for making the megakaryocyte derivatives (e.g., MLCs or PLCs or derivatives thereof or lysates thereof or PRP, PRGF, or AS derived therefrom) of the present disclosure. In some embodiments, a pharmaceutical composition comprises (1) megakaryocyte derivatives (e.g., MLCs, PLCs or derivatives thereof or lysates thereof or PRP, PRGF, or AS derived therefrom) or precursor cells for making megakaryocyte derivatives (e.g., MLCs, PLCs or derivatives thereof or lysates thereof or PRP, PRGF, or AS derived therefrom), and (2) a pharmaceutically acceptable bulking agent, carrier or excipient. In some embodiments, a pharmaceutical composition comprises (1) megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom), (2) a pharmaceutically acceptable bulking agent, carrier or excipient, and optionally, (3) at least one additional therapeutic agent. In some embodiments, a pharmaceutical composition comprises (1) the megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom), (2) a pharmaceutically acceptable bulking agent, carrier or excipient, and optionally, (3) at least one additional therapeutic agent and an enriching agent.

The composition comprising the megakaryocyte derivatives (e.g., MLCs, PLCs or derivatives thereof or lysates thereof or PRP, PRGF, or AS derived therefrom) could advantageously be enriched with or formulated with one or more of a polysaccharide, e.g., a natural polysaccharide (such as hyaluronic acid, hydroxypropylcellulose, karya gum (KG), guar gum (GUG), or gellan gum (GEG)), dermal fillers (such as, Juvéderm®, Juvéderm® Ultra Plus, Perlane®, Belotero®, Restylane®), a semi-synthetic polysaccharide or a synthetic polysaccharide, and a synthetic polymer, (e.g., poly(7-oxanorbornene-2-carboxylate), F127 or poly(lactic-co-glycolic acid) (PLGA)), sodium citrate, calcium chloride, proteoglycans, adenine, guanine, cytosine, thiamine, progenitor stem cells or their derivatives, vitamins, retinols, retinoic acid, retinol palmitate, acetate (e.g.) tocoferil acetate, a phosphate (e.g., sodium ascorbyl phosphate), D-panthenol, peptides, recombinant growth factors, micronized human-identical hormones, amino acids, phyto-extracts, anti-oxidants, lipoic acid, DMAE, collagen, GAG, trace elements, minerals, proteases, ceramides, polysaccharides, algae, marine extracts, monocytes or a combination thereof.

Additional therapeutic agents include, but are not limited to, those described elsewhere in the present disclosure. The composition comprising the megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom) can be administered locally (e.g., osteoarthritis knee), parenterally, i.e., infusion, subcutaneous, intramuscular, intravenous, intradermal, intrathecal, and epidural, or topically, optically, or by inhalation, or rectally, or vaginal, or sub-lingually.

Therapeutic compositions and formulation thereof comprising megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom), used in accordance with the present disclosure, are prepared for storage by mixing the megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or PRP, PRGF, or AS derived therefrom), having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG), Other pharmaceutically acceptable carriers can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.), water, salt solutions, alcohols, polyethylene glycols, gelatins, amyloses, magnesium stearates, talcs, silicic acids, viscous paraffins, hydroxymethylcelluloses, polyvinylpyrrolidones and the like. Pharmaceutical formulations to be used for in vivo administration are generally sterile. This is readily accomplished by filtration through sterile filtration membranes.

The formulations comprising the compositions described herein comprise granules, tablet, suspension in a liquid carrier, capsule or powder. The formulations herein may also contain more than one active compound as necessary for the particular indication being treated, preferably those with complementary activities that do not adversely affect each other. For example, in addition to the megakaryocyte derivatives (e.g., MLCs or PLCs or derivatives thereof or lysates thereof or PRP, PRGF, or AS derived therefrom) or precursor cells for making the megakaryocyte derivatives (e.g., MLCs, PLCs or derivatives thereof or PRP, PRGF, or AS derived therefrom), it may be desirable to include in the one formulation, an additional injury healing agent, e.g., an anti-inflammatory agent or an opioid drug. Alternatively, or additionally, the composition may further comprise a cytotoxic agent, cytokine, growth inhibitory agent, anti-hormonal agent, and/or cardioprotectant. Such molecules are suitably present in combination in amounts that are effective for the purpose intended.

The active ingredients (i.e., MLCs or PLCs or derivatives thereof or lysates thereof, or PRP, PRGF, or AS derived therefrom) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

The active ingredients (i.e., MLCs, PLCs or derivatives thereof or lysates thereof or PRP, PRGF, or AS derived therefrom) may also be delivered in bioactive scaffolds made from natural (e.g., protein based) or synthetic (polymer or ceramic based) biomaterials. The bioactive scaffolds, in addition to the megakaryocyte derivatives (e.g., MLCs, PLCs or derivatives thereof or lysates thereof or the PRP, PRGF, or AS derived therefrom) may optionally contain growth factors and other bioactive molecules. Among these are epidermal growth factor. TGF-alpha, TGF-beta, fibroblast growth factor, platelet derived growth factor, vascular endothelial growth factor, insulin-like growth factor, keratinocyte growth factor, and bone morphogenic protein to name a few. Growth factors may also be introduced into the scaffolds before or after the introduction of the megakaryocyte derivatives (e.g., MLCs, PLCs or derivatives thereof or lysates thereof or the PRP, PRGF, or AS derived therefrom).

Sustained-release preparations may be prepared. Suitable examples of sustained-release preparations include semi-permeable matrices of solid hydrophobic polymers containing the megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. Biocompatible materials that may be present in a hydrogel include, e.g., permeable configurations or morphologies, such as polyvinyl alcohol, polyvinylpyrrolidone and polyacrylamide, polyethylene oxide, poly(2-hydroxyethyl methacrylate); natural polymers such as polysaccharides, gums and starches; and include poly[alpha (4-aminobutyl)]-1-glycolic acid, polyethylene oxide, polyorthoesters, silk-elastin-like polymers, alginate, EVAc (poly(ethylene-co-vinyl acetate), microspheres such as poly (D, L-lactide-co-glycolide) copolymer and poly (L-lactide), poly(N-isopropylacrylamide)-b-poly(D,L-lactide), a soy matrix such as one cross-linked with glyoxal and reinforced with a bioactive filler, e.g., hydroxylapatite, poly(epsilon-caprolactone)-poly(ethylene glycol) copolymers, poly(acryloyl hydroxyethyl) starch, polylysine-polyethylene glycol, or agarose. In one embodiment, the hydrogel includes poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof. In some embodiments, the hydrogel includes collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxideypoly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS). The following polymers may be employed, e.g., natural polymers such as alginate, agarose, starch, fibrin, collagen, gelatin, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone. While polymers such as ethylene-vinyl acetate and lactic acid-glycolic acid enable release of molecules for over 100 days, certain hydrogels release proteins for shorter time periods. When encapsulated megakaryocyte derivatives (e.g., MLCs or PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom) remain in the body for a long time, they may denature or aggregate as a result of exposure to moisture at 37 degree c., resulting in a loss of biological activity and possible changes in activity.

Megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom) may be formulated in any suitable form for delivery to a target cell/tissue. For example, megakaryocyte derivatives (e.g., MLCs or MLC derivatives or PLCs or derivatives thereof or lysates thereof or PRP, PRGF, or AS derived therefrom) or precursor cells for making the megakaryocyte derivatives (e.g., MLCs, PLCs or derivatives thereof or PRP, PRGF, or AS derived therefrom) may be formulated as immunoliposomes. A “liposome” is a small vesicle composed of various types of lipids, phospholipids and/or surfactant which is useful for delivery of a drug to a mammal. The components of the liposome are commonly arranged in a bilayer formation, like the lipid arrangement of biological membranes. Liposomes containing the megakaryocyte derivatives (e.g., MLCs, PLCs or derivatives thereof or lysates thereof or PRP, PRGF, or AS derived therefrom) are prepared by methods known in the art, such as described in Epstein et al., Proc. Natl. Acad. Sci. USA 82:3688 (1985); Hwang et al., Proc. Natl. Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Another therapeutic agent is optionally contained within the liposome. See Gabizon et al., J. National Cancer Inst. 81(19):1484 (1989).

The formulations to be used for in vivo administration must be sterile. This is readily accomplished by filtration through sterile filtration membranes.

For treating injuries (e.g., OA, regenerating bone tissue to influence the process of bone regeneration and repair) or to regenerate aging skin or hair, in one embodiment, the megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom) are administered via any of the routes disclosed herein. The dosage administered via any of these routes is in the range of about 0.1 microgram/m² to about 10,000 microgram/m² per dose, generally one dose per day or per week for a total of one, two, three or four doses or multiple doses depending on the need. Alternatively, the dosage range is of about 0.1 microgram/m² to about 1000 microgram/m², about 0.1 microgram/m² to about 800 microgram/m², about 0.1 microgram/m² to about 600 microgram/m², about 0.1 microgram/m² to about 400 microgram/m², about 0.1 microgram/m² to about 500 microgram/m², about 0.1 microgram/m² to about 300 microgram/m², about 0.1 microgram/m² to about 200 microgram/m², and about 0.1 microgram/m² to about 200 microgram/m². The dose may be administered once per day, once per week, multiple times per week, less than once per day or more than once per day, 2-3 times per day, multiple times per month, once per day, once per week or once per month or intermittently on a daily, weekly or a monthly basis (e.g., one or more than one dose is administered, daily, weekly, biweekly, triweekly or monthly) to relieve or alleviate symptoms of the injury or aging skin or disease related to the injury or aging skin. Administration may continue at any of the disclosed intervals until remission of the injury or aging of skin or symptoms related to the disease are being treated. Administration may continue after remission or relief of symptoms is achieved where such remission or relief is prolonged by such continued administration.

Emulsifying agents can natural or synthetic and include, but are not limited to, Cationic, e.g., benzalkonium chloride, benzethonium chloride. Anionic, e.g., alkali soaps (sodium or potassium oleate); amine soaps (triethanolamine stearate); detergents (sodium lauryl sulfate, sodium dioctyl sulfosuccinate, sodium docusate). Nonionic, e.g., sorbitan esters (Spans®), polyoxyethylene derivatives of sorbita esters (Tweens®), or glyceryl esters

Other agents that can be used as emulsifiers may include desoxycholic acid, diacetyl tartaric acid esters, egg yolk, glycerol, Gums, Irish Moss (carrageenan), Lecithin, Mono- and diglycerides, Monosodium phosphate, Monostearate, Ox bile extract, Propylene glycol, Soaps, Taurocholic acid (or its sodium salt).

In some embodiments of the present disclosure, megakaryocyte derivatives (e.g., MLCs, PLCs or derivatives thereof or lysates thereof or the PRP, PRGF, or AS derived therefrom) or precursor cells for making the megakaryocyte derivatives (e.g., MLCs, PLCs or derivatives thereof or PRP, PRGF, or AS derived therefrom) are in a plasmalyte. Plasmalyte is a family of balanced crystalloid solutions. It closely mimics human plasma in its content of electrolytes, osmolality, and pH. These solutions also have additional buffer capacity and contain anions such as acetate, gluconate, and even lactate that are converted to bicarbonate, CO₂, and water.

In some embodiments, MKs and platelets can also be derived from human induced pluripotent stem cells (iPSCs), embryonic stem cells (ESCs), immortalized megakaryocytic progenitor cells, hematopoietic stem cells, including but not limited, to CD34⁺ umbilical cord blood stem cells (UCB cells) (e.g., human CD34⁺ umbilical cord blood stem cells) such as those disclosed in a co-pending U.S. patent application Ser. No. 16/975,918, filed Jun. 25, 2020 and PCT Application based on U.S. Provisional application No. 63/025,209, filed May 15, 2020, incorporated herein in their entireties by reference, CD34⁺ mobilized peripheral blood cells (MPB cells) (e.g., CD34⁺ human mobilized peripheral blood), or CD34⁺ bone marrow cells. UCB cells are multipotent stem cells derived from blood that remains in the placenta and the attached umbilical cord after childbirth. MPB cells are multipotent stem cells derived from volunteers whose stem cells are mobilized into the bloodstream by administration of G-CSF or similar agent.

In some embodiments, MKs and platelets can be derived from other stem cell types, including but not limited to mesenchymal stem cells (MSC) (such as, adipose-derived mesenchymal stem cells (AdMSC)) or mesenchymal stem from other sources.

AdMSCs are derived from white adipose tissue, which is derived from the mesoderm during embryonic development and is present in every mammalian species, located throughout the body. Due to their wide availability and ability to differentiate into other tissue types of the mesoderm-including bone, cartilage, muscle, and adipose-ASCs may serve a wide variety of applications.

In the present disclosure, the stem cell cultures can be maintained independently of embryonic fibroblast feeder cells and/or animal serum. In some embodiments, serum-free, feeder-cell free alternatives can be utilized in the instant methods.

### Extracellular Vesicles (EVs)

In some embodiments, the present disclosure comprises microvesicles and exosomes (collectively referred to as extracellular vesicles (EVs)) or derivatives thereof, which are produced admixtures of the MLCs or PLCs or derivatives thereof or lysates thereof. Given that EVs or derivatives thereof carry growth factors, receptors, bioactive lipids, nucleic acids, such as mRNA and microRNA (miRNA) or siRNA, proteins, they are able to deliver important payloads to recipient cells (e.g., an osteoarthritis knee or to skin or a damaged organ or tissue or for use in regenerative medicine) making it even a richer resource to complement the MLCs or PLCs or derivatives thereof or lysates thereof with additional growth factors or other molecules for treating or ameliorating diseases, disorders, or injuries related to dry eye, osteoarthritis, tendon, ligament, bone repair, wound healing or wound-healing related disorders, alopecia or in skin rejuvenation or regeneration, for example.

EVs or derivatives thereof of the present disclosure can be isolated and purified, essentially separating them from an admixture comprising the MLCs or PLCs or lysates thereof of the present disclosure. Isolated or purified extracellular vesicles (EVs) or derivatives thereof, because of their ability to extensively travel throughout the body, can exert remarkable therapeutic effects when administered to a patient on their own. EVs have a fundamental immunomodulatory potential for treating or inhibiting diseases or disorders, or injuries related to dry eye, osteoarthritis, tendon, ligament, bone repair, wound healing or wound-healing related disorders, alopecia or in skin rejuvenation or regeneration. EVs could also be used as drug delivery system; they are able to cross biological barriers, including the blood-brain barrier and synovial membrane.

Advantageously, EVs or derivatives thereof can be internalized by recipient cells following receptor-ligand interactions and the varied assortment of bioactive molecules, derived from the cell of origin, such as proteins, bioactive lipids, and nucleic acids, can be transferred along with the proteins expressed on the EV surface.

In some embodiments, EVs or derivatives thereof may directly activate a recipient cell (e.g., donor platelets) by acting as signaling complexes. For example, EVs or derivatives thereof may bind to platelets by means of the P-selectin glycoprotein ligand-1 expressed on their surface and EVs or derivatives thereof from neutrophils expressing Mac-1 may induce donor platelet activation in a patient in need thereof. Such activations are advantageous because they facilitate activation of exogenous mechanisms that can enhance treatments related to the treating or ameliorating diseases, disorders, or injuries related to dry eye, osteoarthritis, tendon, ligament, bone repair, wound healing or wound-healing related disorders, alopecia or in skin rejuvenation or regeneration.

Compositions and methods comprising the extracellular vesicles (EV) or derivatives thereof of the present disclosure can be used in several therapies or co-therapies, such as for treating or ameliorating diseases, disorders, or injuries related to dry eye, osteoarthritis, tendon, ligament, bone repair, wound healing or wound-healing related disorders, alopecia or in skin rejuvenation or regeneration, delivery of genes, proteins or peptides, nucleic acids for the use in cellular or gene therapies, for example using vectors, e.g. adenovirus, lentivirus, to obtain novel microvesicles or exosomal gene (e.g., for gene therapy), peptide (for growth factors) or nucleic acid (e.g., siRNA or microRNA) delivery vehicles. Packaging within extracellular vesicles (EV) provides several advantages such as shielding the molecules from adverse cellular event that may neutralize the naked gene. Engineered extracellular vesicles (EV) could be used to carry drugs to specific sites of tissue damage, including but not limited to dry eye, osteoarthritis, tendon, ligament, bone repair, wound healing or wound-healing related disorders, alopecia or in skin rejuvenation or regeneration.

In some embodiments, the isolated extracellular vesicles (EV) derivatives thereof may then be stored until use by freezing at very low temperature, e.g., at −80° C. in presence of cryopreserving agents, such dimethylsulphoxide (DMSO) and glycerol used at optimal concentrations.

In some embodiments, an average diameter of extracellular vesicles (EV) derived from a population of iPSC derived platelets is less than 50% the diameter of the extracellular vesicles (EV) derived from a population of donor derived platelets having about the same number of platelets as the population of iPSC derived platelets. In some embodiments, the megakaryocyte or platelet is genetically modified to comprise a nucleic acid molecule encoding a therapeutic agent.

Extracellular vesicles (EV) are subcellular sized particles consisting of a membrane lipid bilayer and cellular content. Extracellular vesicles (EV) isolated or purified from an admixture comprising MLCs, PLCs, lysates thereof or combination thereof may exert both anti-inflammatory and pro-inflammatory function and have potential as vehicles for drug delivery.

In some embodiments, the diameter of the instant extracellular vesicles (EV) is 0.1 and 4 μm. In some embodiments, the diameter of the instant extracellular vesicles (EV) is 0.1 and 3 μm. In some embodiments, the diameter of the instant extracellular vesicles (EV) is 0.1 and 2.5 μm. In some embodiments, the diameter of the instant extracellular vesicles (EV) is 0.1 and 2 μm. In some embodiments, the diameter of the instant extracellular vesicles (EV) is 0.1 and 1.5 μm. In some embodiments, the diameter of the instant extracellular vesicles (EV) is 0.1 and 1.0 μm. In some embodiments, the diameter of the instant extracellular vesicles (EV) is 0.1 and 0.9 μm. In some embodiments, the diameter of the instant extracellular vesicles (EV) is 0.1 and 0.8 μm. In some embodiments, the diameter of the instant extracellular vesicles (EV) is 0.1 and 0.7 μm. In some embodiments, the diameter of the instant extracellular vesicles (EV) is 0.1 and 0.6 μm. In some embodiments, the diameter of the instant extracellular vesicles (EV) is 0.1 and 0.5 μm. In some embodiments, the diameter of the instant extracellular vesicles (EV) is 0.1 and 0.4 μm. In some embodiments, the diameter of the instant extracellular vesicles (EV) is 0.1 and 0.3 μm. In some embodiments, the diameter of the instant extracellular vesicles (EV) is 0.1 and 0.2 μm. In some embodiments, the diameter of the instant extracellular vesicles (EV) is 0.2 and 1 μm. In some embodiments, the diameter of the instant extracellular vesicles (EV) is 0.3 and 1 μm. In some embodiments, the diameter of the instant extracellular vesicles (EV) is 0.4 and 1 μm. In some embodiments, the diameter of the instant extracellular vesicles (EV) is 0.5 and 1 μm. In some embodiments, the diameter of the instant extracellular vesicles (EV) is 0.6 and 1 μm. In some embodiments, the diameter of the instant extracellular vesicles (EV) is 0.7 and 1 μm. In some embodiments, the diameter of the instant extracellular vesicles (EV) is 0.8 and 1 μm. In some embodiments, the diameter of the instant extracellular vesicles (EV) is 0.9 and 1 μm. In some embodiments, the diameter of the instant extracellular vesicles (EV) is 0.2 and 2 μm. In some embodiments, the diameter of the instant extracellular vesicles (EV) is 0.3 and 2 μm. In some embodiments, the diameter of the instant extracellular vesicles (EV) is 0.4 and 2 μm. In some embodiments, the diameter of the instant extracellular vesicles (EV) is 0.5 and 2 μm. In some embodiments, the diameter of the instant extracellular vesicles (EV) is 0.6 and 2 μm. In some embodiments, the diameter of the instant extracellular vesicles (EV) is 0.7 and 2 μm. In some embodiments, the diameter of the instant extracellular vesicles (EV) is 0.8 and 2 μm. In some embodiments, the diameter of the instant extracellular vesicles (EV) is 0.9 and 2 μm. In some embodiments, the diameter of the instant extracellular vesicles (EV) is 1.0 and 2 μm. In some embodiments, the diameter of the instant extracellular vesicles (EV) is 1.5 and 2 μm. In some embodiments, the diameter of the instant extracellular vesicles (EV) is 2.0 and 2.5 μm.

Extracellular vesicles (EV) derivatives thereof may be conjugated to one or more cytotoxic agents by mechanisms disclosed in the foregoing. One or more cytotoxic agents may be imbibed into the extracellular vesicles (EV) derivatives thereof by mechanisms also disclosed in the foregoing. Cytotoxic agents are also disclosed in the foregoing. Diseases and disorders that can be cured or mitigated by the use of EVs derivatives thereof alone or in combination with the MLCs, PLCs, lysates thereof or derivatives thereof of the present disclosure are also disclosed below.

In some embodiments, EVs, whether modified or not (e.g., bioengineered or conjugated) may be developed for therapeutic use independent of the MLCs, PLCs, lysates therefrom or derivatives therefrom. For example, a patient in need of a treatment predominantly involving microvesicles or derivatives thereof will be administered microvesicle-based treatment or exosome-based treatment or a combination of both. For example, MVs or exosomes incorporated with exogenous growth factors, cytokines, or siRNAs can be used for efficient silencing of a target MAPK gene in monocytes and lymphocytes or deliver growth factors siRNAs (e.g., VEGF-siRNA) targeting, for example, regenerating bone tissue to influence the process of bone regeneration and repair and alopecia. Advantageously, MVs could be used as more efficient delivery vehicles to direct specific targeting of novel therapeutics without immunogenicity and adverse effects.

In some embodiments, the EV-based treatment may be administered prior to treatment with the megakaryocyte derivatives (e.g., MLC, MLC lysate, PLC or PLC lysate). In some embodiments, treatment with the megakaryocyte derivatives (e.g., MLC, MLC lysate, PLC or PLC lysate) may be administered prior to EV-based treatment. In some embodiments, megakaryocyte derivatives (e.g., MLCs, PLCs, lysates thereof, or combinations thereof) and EVs are administered as admixtures. Also contemplated are treatments in which admixtures comprising megakaryocyte derivatives (e.g., MLCs, PLCs, lysates thereof, or combinations thereof) and EVs are administered followed by treatment regiments comprising essentially of EV or derivatives thereof or comprising essentially of megakaryocyte derivatives (e.g., MLCs or PLCs or derivatives thereof or lysate thereof-based treatment) based treatment depending on a patient's need.

Some embodiments of the present disclosure provide a composition suitable for cryopreservation including the megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or PRP, PRGF, or AS derived therefrom) and a cryopreservation agent. In some embodiments, the cryopreservation agent is a sugar, alcohol, polymer, protein, or a combination thereof. In some embodiments, the cryopreservation agent is DMSO, glycerol, trehalose, cellulose, or a combination thereof. In some embodiments, the cryopreservation agent is DMSO. In some embodiments, the cryopreservation agent is glycerol or arabinogalactan. In some embodiments, the cryopreservation agent is DMSO and glycerol. In some embodiments, the cryopreservation agent is trehalose or propylene glycol or albumin or a combination thereof. In some embodiments, the cryopreservation agent is cellulose. A combination comprising one or more of the cryopreservation agents is also encompassed in the present disclosure. In some embodiments, the composition is frozen, i.e., the composition is stored at a temperature between about −80° C. and −200° C. Cryoprotective agents can be classified as penetrating and non-penetrating. Non-penetrating cryoprotective agents alter only the freezing characteristics of the extracellular medium whereas penetrating cryoprotective agents can modify both the intracellular and the extracellular medium composition.

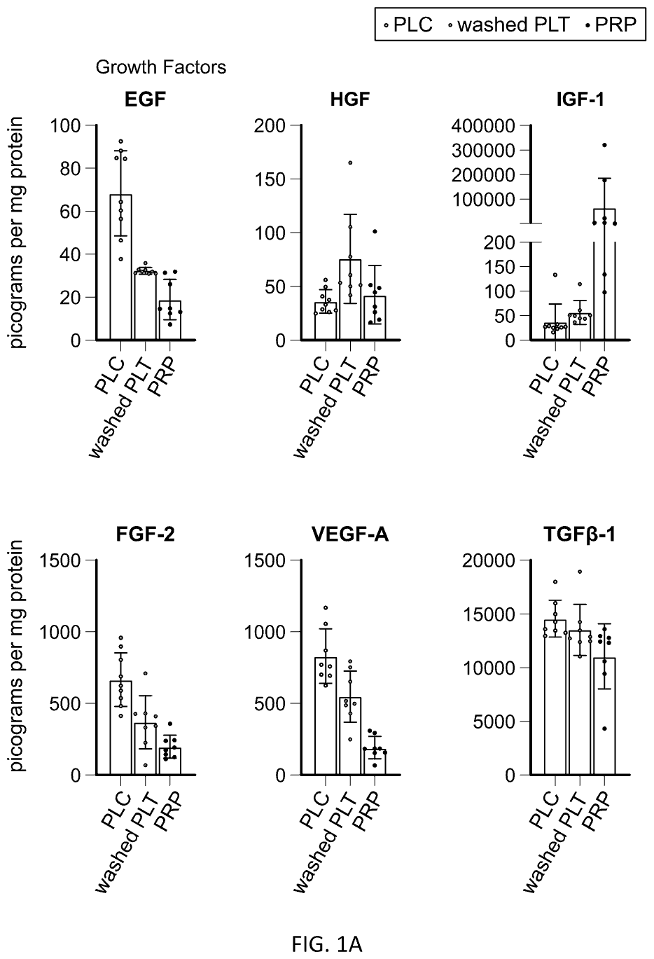

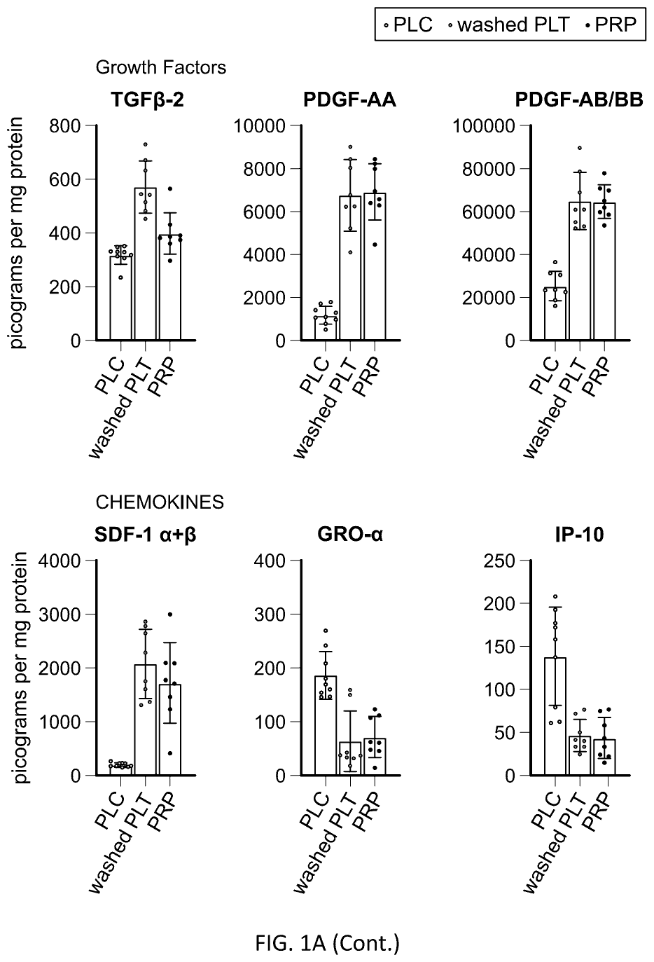

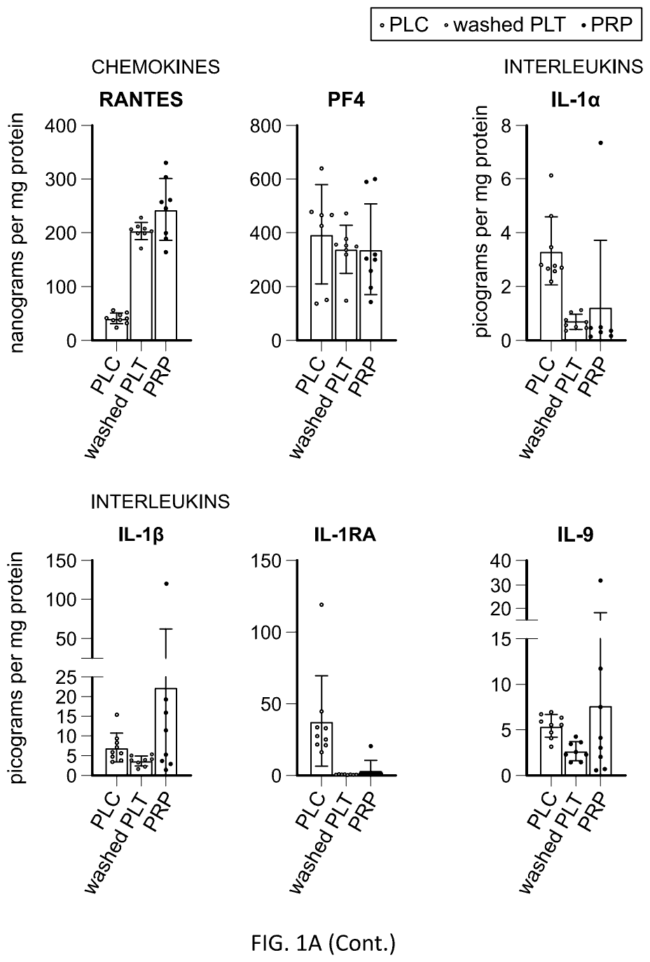

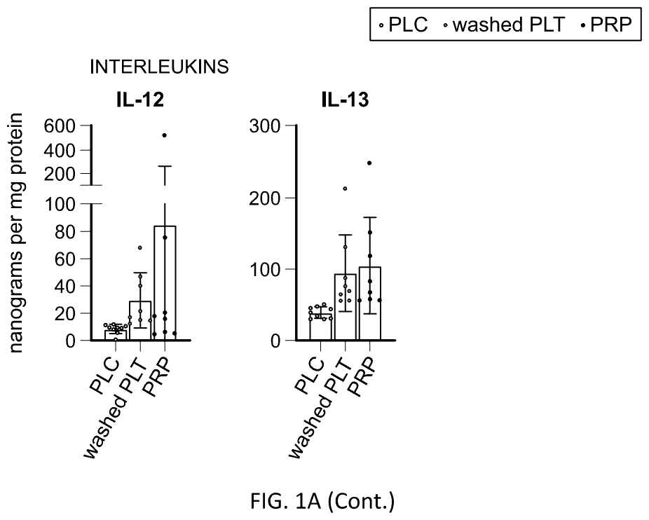

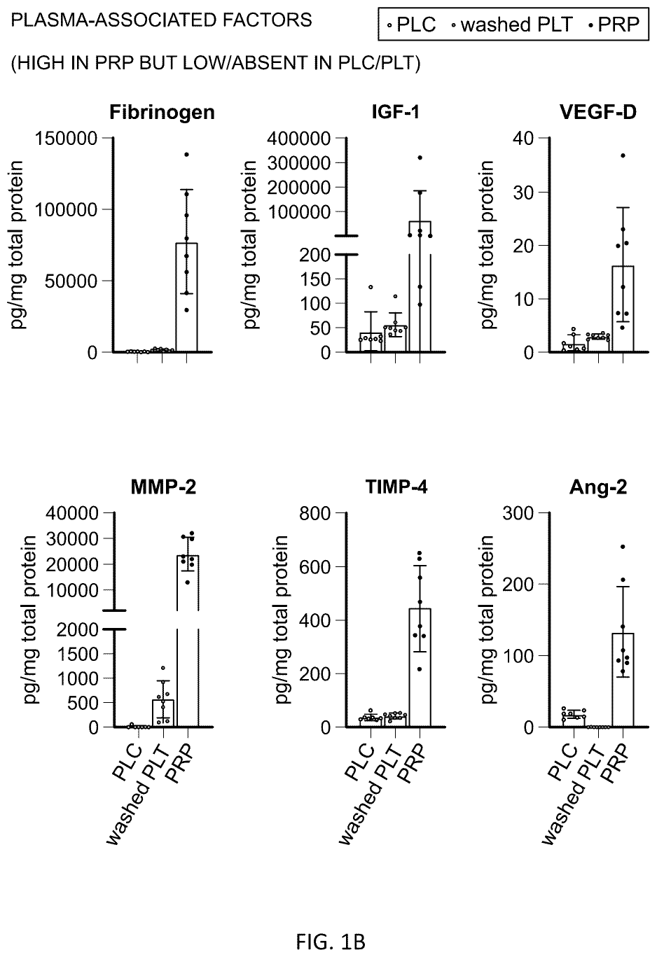

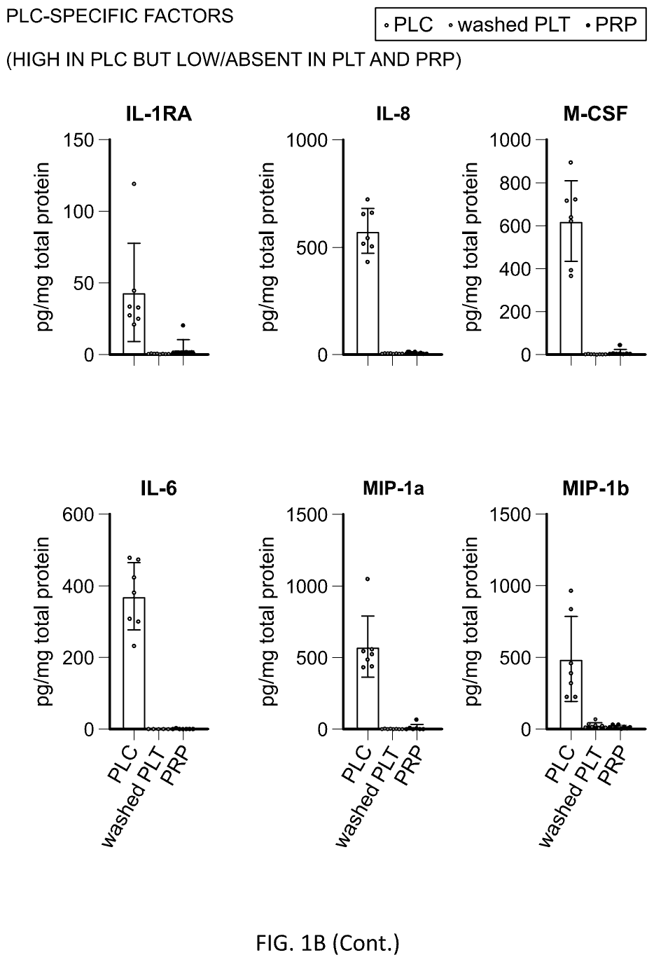

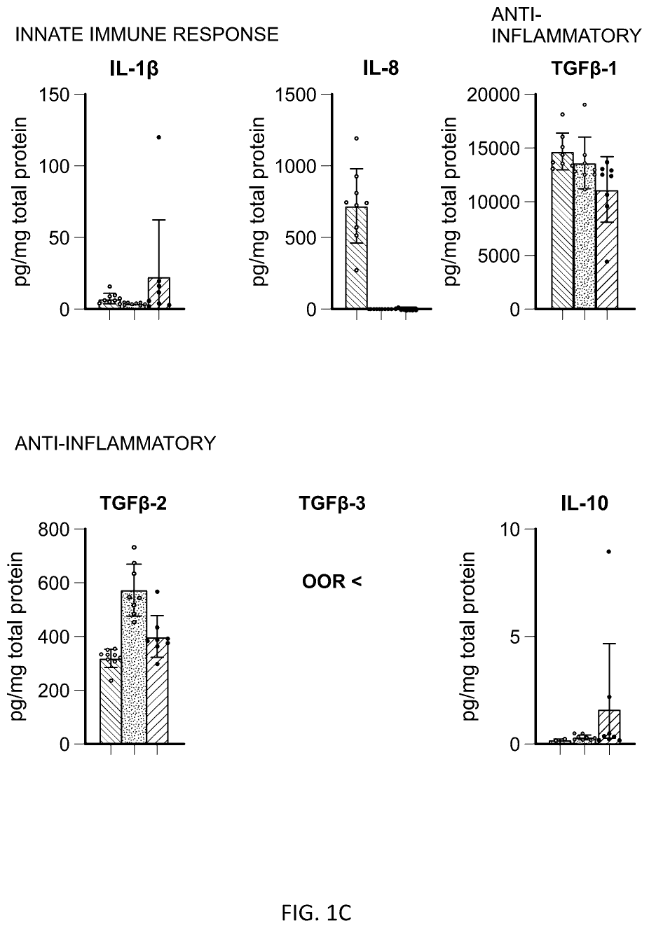

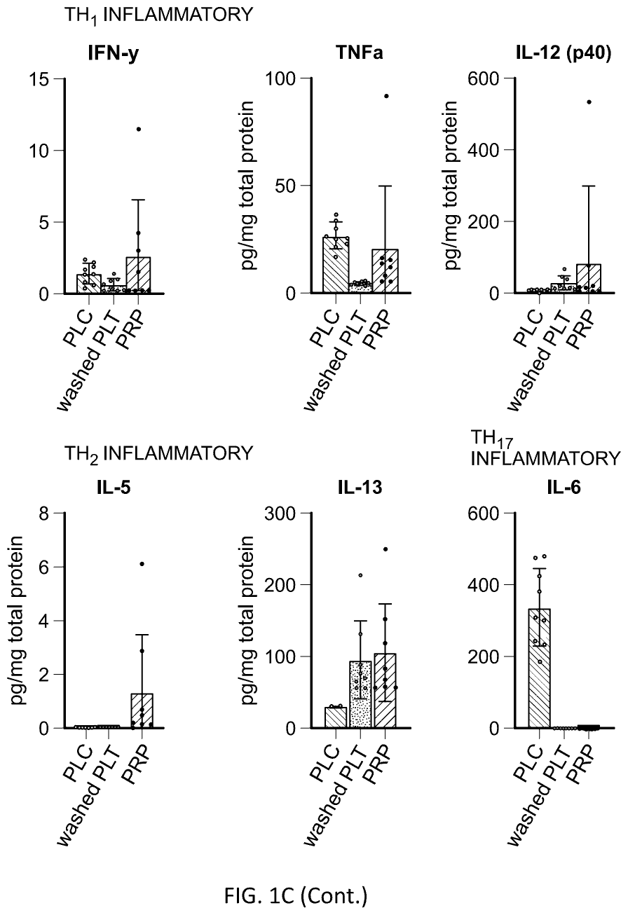

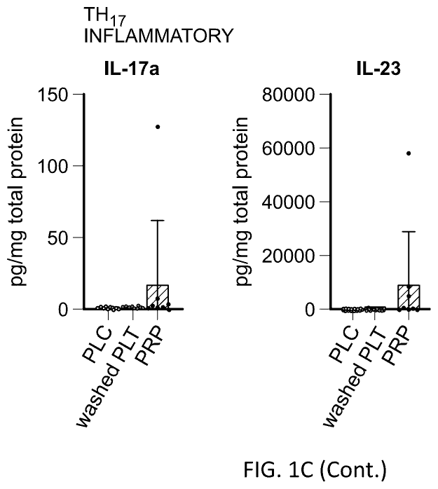

The megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or PRP, PRGF, or AS derived therefrom), in a non-limiting embodiment, are rich in growth factors or could be supplemented with growth factors, such as but not limited to Angiopoietin-1, bFGF, EGF, FGF (e.g., FGF-2), HGF, IGF-I, IGF-II, PDAF, PDEGF, PDGF (e.g., PDGF-AA, PDGF-BB, PDGF-AA/BB), TGF-beta (e.g., TGF-β1, TGF-β2, and TGF-β3), VEGF (e.g., VEGF-A, VEGF-C), IFN-γ, cytokines (e.g., IL-1B, IL-2, IL-4, IL-5, IL-6, IL-8, IL-10, IL-12, IL12p40, IL-12p70, IL-13, IL-17A, IL-23, TNF-A, MIG, MCP-1, IP-10), chemokines (e.g., ENA-78 (CXCL5), IL-8 (CXCL8), monocyte chemotactic protein (e.g., MCP-3 (CCL7), MIP-1A (CCL3), NAP-2 (CXCL7), PF4 (CXCL4), or inflammatory mediators (e.g., PGE2), macrophage inflammatory protein-1 (MIP-1) or regulated on activation, normal T cell expressed and secreted (RANTES), their concentrations often higher than that obtained from donor platelets. The concentrations of the growth factors or cytokines in PRP, PRGF, or AS enriched from MLCs, PLCs or derivatives thereof or lysates thereof or PRP, PRGF, or AS derived therefrom or MLCs, PLCs or derivatives thereof (e.g., lysates from MLCs, PLCs or derivatives therefrom) or precursor cells for making the MLCs, PLCs or derivatives thereof or PRP, PRGF, or AS derived therefrom may be diluted as desired by a subject's need. In some embodiments, MLCs, PLCs can be genetically engineered to express one or more of the growth factors discussed herein. Non-limiting examples of endogenous PLC-based growth factors are shown in FIGS. 1A-1C (n=8 each). FIG. 1A provides growth factor and cytokine profile in PLCs as compared to donor PRP and washed donor platelets. FIG. 1B provides the factors that are most different between PLCs as compared to donor PRP and washed donor platelets. FIG. 1C provides a summary of pro/anti-inflammatory markers relevant for dry eye disease.

megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or PRP, PRGF, or AS derived therefrom) or a composition thereof can be administered or applied to damaged tissues of the musculoskeletal system, such as sprained ligaments (which attach bone to bone), strained or torn muscles can be torn, tendon ruptures, osteoarthritis, fractured bones can be cracked or broken or the surrounding injured tissues, bone dislocations, i.e., where the bones in a joint may become completely separated from each other (called dislocation) or only partly out of position (called subluxation), sprains, strains, or other musculoskeletal injuries, completely or partially tom tendons, muscle spasms, nerve pains, whiplash, athletic injuries, myocardial infarction or ischemia-related disorders (e.g., limb ischemia, lower extremity ischemia, myocardial ischemia, organ ischemia, ischemic heart disease or the like), surgical procedures, which are difficult to heal, lung diseases, cardiac diseases,

The site of a tissue damage or injury may be determined by well-established techniques, such as but not limited to, imaging studies which include, but are not limited, to MRI, X-ray, CT scan, X-rays which may be done to check for fractures and dislocations, which may also be present. In addition, x-rays can show abnormalities in the position of bones that may suggest a sprain or other soft-tissue injury. Magnetic resonance imaging (MRI), MRI can show soft tissues, which are not usually visible on x-rays. MRI thus helps detect injury to tendons, ligaments, cartilage, and muscle. Other techniques, such as but not limited to, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging NOGA mapping and ultrasound techniques may also be applied to determine a site of injury. A physician may consult a subject before one or more of the above testing is performed.

The megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or PRP, PRGF, or AS derived therefrom or a composition thereof) may be administered through different routes, such as but not limited to, systemic, topical, with the use of an implantable device, such as but not limited to stents, mesh (e.g., a polymer mesh or a bioabsorbable mesh), adhesive biomaterials (e.g., naturally derived or synthetic biopolymers) or other devices know to one of skill in the art. For example, variables such as proper timing, treatment periodicity, location and technique for knee injection (e.g., an intravenous or intradermal injection) in patients suffering from knee osteoarthritis treatment may vary from a single or multiple megakaryocyte derivatives (e.g., MLC, PLC, derivative thereof or lysate thereof) applications. megakaryocyte derivatives (MLCs or PLCs or derivatives therefrom or lysate therefrom) can be applied on a daily, weekly (3-weekly PLC injections) or monthly basis (two monthly injections, three injections at 15-day intervals or 21-day intervals. Treatment regimens may vary depending on the need of a patient). Location and technique for the application of the megakaryocyte derivatives (e.g., MLCs, PLCs or derivatives thereof or lysates thereof or PRP, PRGF, or AS derived therefrom) (e.g., local injection to a knee) could be lateral, supero-lateral, para-patellar, and lateral mid-patellar, among others. In each such treatment, optionally one or more of other therapeutic agents can be co-administered simultaneously, or at periodic intervals from one another.

If stents are used, the stents could be closed cell or open cell stents, which are well known to one of skill in the art. megakaryocyte derivatives (e.g., MLCs, PLCs or derivatives thereof or lysates thereof or PRP, PRGF, or AS derived therefrom) may be bonded directly to a metal stent or be bonded to a matrix polymer, which acts as a drug reservoir to ensure source retention during deployment and a uniform distribution on the stent. The types, compositions, and designs of the polymers coated on the stent generally will dictate the eluting kinetic of the sustain time release of the megakaryocyte derivatives (e.g., MLCs, PLCs or derivatives thereof or lysates thereof or PRP, PRGF, or AS derived therefrom) over a period of weeks or months following the implantation in situ. The coating materials can be categorized as organic vs inorganic, bioerodable vs nonbioerodable, and synthetic vs naturally occurring substances.

In some embodiments, the composition comprising the megakaryocyte derivatives (e.g., MLCs or PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom) comprises between 0.01 and 1 wt. %, 1 and 25 wt. %, 25 and 50 wt. %, 50 and 75 wt. %, 75 and 100 wt. % of megakaryocyte derivatives (e.g., MLCs or PLCs or derivatives thereof or lysates therefrom) or platelet-rich plasma (PRP), PRGF, plasma, serum, or other blood derivative derived therefrom.

In some embodiments, the composition comprises between 1 and 25 wt. %, 25 and 50 wt. %, 50 and 75 wt. %, 75 and 98 wt. % of a bulking agent. In some embodiments, the composition comprises between 1 and 25 wt. %, 25 and 50 wt. %, 50 and 75 wt. %, 75 and 98 wt. % of at least one excipient or carrier. Once the wt. % of megakaryocyte derivatives (e.g., MLCs or PLCs or derivatives thereof or lysates thereof) or platelet-rich plasma (PRP), PRGF, plasma, serum, or other blood derivative derived therefrom, bulking agent, excipient or carrier can be adjusted accordingly. For example, in an aspect of the present disclosure the composition may comprise a) between 1 and 50 wt. % of megakaryocyte derivatives (e.g., MLCs or PLCs or derivatives thereof or lysates thereof) or platelet-rich plasma (PRP), PRGF, plasma, serum, or other blood derivative derived therefrom, b) between 1 and 25 wt. % of a bulking agent, and/or c) between 50 and 98 wt. % of at least one excipient or carrier.

In some embodiments, the adhesive biopolymer materials may include, but are not limited to, polycarbophil (PCP), Xanthan gum, pectin, Hydroxypropyl methylcellulose (HPMC) or hypromellose Carbopol 1342P, Carbopol 974P, Chitosan, Carbopol 971P, hydroxypropylmethyl-cellulose (Methacel K100M), CMC-Na, hydroxypropylmethyl-cellulose (Methacel K15M), gelatin, Acacia gum or a combination thereof. When the MLCs, PLCs or derivatives thereof or lysates thereof or PRP, PRGF, or AS derived therefrom is contained in adhesive biomaterials, the adhesive bioagents adhere to the target sites in order to extend the retention time of the MLCs, PLCs or derivatives thereof or lysates thereof or PRP, PRGF, or AS derived therefrom in a lesion (e.g., ocular lesion), and improve the treatment effect of local disease (e.g., dry eye disease). Higher local drug concentration and the close contact with the site of absorption can not only promote absorption of the drug, but also increase concentration gradient. The adhesive biopolymers may modulate transport pathways by opening epithelial tight junctions to promote the diffusion of the megakaryocyte derivatives (e.g., MLCs, PLCs or derivatives thereof or lysates thereof or PRP, PRGF, or AS derived therefrom). In addition, megakaryocyte derivatives (e.g., MLCs, PLCs or derivatives thereof or lysates thereof or PRP, PRGF, or AS derived therefrom) may adhere to the mucosa directly and is absorbed by the mucosal capillaries to increase the bioavailability. Furthermore, the use of adhesive biomaterials permit in the preparation of controlled release formulations for the megakaryocyte derivatives (e.g., MLCs, PLCs or derivatives thereof or lysates thereof or PRP, PRGF, or AS derived therefrom), which can reduce the frequency of administration to improve a patient's health in need of therapy with the megakaryocyte derivatives (e.g., MLC, PLC, derivative thereof, and/or lysate thereof).

In some embodiments, the megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom) may be applied to dry eye disease. Dry eye diseases fall into two categories, which are (i) tear-deficient and (ii) evaporative. Sjogren's syndrome or non-Sjogren's syndrome causes are inclusive of exocrinopathy in which lacrimal secretion deficiency occurs due to an autoimmune process that affects the lacrimal glands, salivary glands, and other organs of the body (Sjogren's syndrome) or inclusive of Non-Sjogren's syndrome caused by lacrimal diseases or lacrimal obstruction and by reflex alterations, without autoimmune factor role. Some causes of Sjogren's syndrome or non-Sjogren's syndrome are age-related dry eye, congenital alacrima, familial dysautonomia, sarcoidosis, lymphoma, AIDS (acquired immunodeficiency syndrome), gland denervation, lacrimal obstruction as in pemphigus, trigeminal injury, diabetes, neurotrophic keratopathy, use of contact lenses, and motor reflex block due to VII pair injury. The evaporative causes of dry eye disease are due to oil deficit, lid changes, use of contact lenses, or ocular surface diseases, as allergic conjunctivitis, and some of the iatrogenic dry eye that occurs after the use of systemic or topical medications or after surgeries or nonsurgical procedures.

The megakaryocyte derivatives (e.g., MLCs, PLCs or derivatives thereof or lysates thereof PRP, PRGF, or AS derived therefrom) or precursor cells for making the megakaryocyte derivatives (e.g., MLCs, PLCs or derivatives thereof or PRP, PRGF, or AS derived therefrom) may be applied in eyedrops. For example, two to three milliliters of this concentrate may be placed in sterile eyedrops. The eyedrops can kept at −20° C. for long term storage. When the use of the megakaryocyte derivatives (e.g., MLCs, PLCs or derivatives thereof or lysates thereof or PRP, PRGF, or AS derived therefrom) containing eye drop is desired, a patient may thaw the eye drops and keep it at +4° C. The patients may use these eyedrops one to six times a day for 1 month, 2 months, 3 months or more. In some embodiments, the patient may use the eye drops once a day for 1 weak, 2 weeks, 3 weeks, or four weeks or more.

In some embodiments, the megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or PRP, PRGF, or AS derived therefrom) may be applied via an injection or topically to areas of skin such as aging skins in the face, scalp, neck, chest, hands, arms, legs, abdomen, or buttocks. It is envisioned that the megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or PRP, PRGF, or AS derived therefrom) may be applied to scalp to prolong the life of hair follicles and increase hair growth. For use on skin or the scalp, megakaryocyte derivatives (e.g., MLCs or PLCs or derivatives thereof or lysates thereof or PRP, PRGF, or AS derived therefrom) or the precursor cells for making the megakaryocyte derivatives (e.g., MLCs, PLCs, lysates thereof or derivatives thereof or PRP, PRGF, or AS derived therefrom) are provided in a composition comprising cosmetically acceptable carrier. For example, in an aspect of the present disclosure is provided a method for generating a hair follicle in a scalp or hair-loss region thereof of a subject comprising contacting the scalp or a hair-loss region therein with the megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or PRP, PRGF, or AS derived therefrom). Another precursor cell that is capable of differentiation into a hair follicle cell may also be included therewith such that the precursor cell is an inductive cell that is capable of inducing differentiation of an uncommitted epidermal cell into a hair follicle cell when combined with the megakaryocyte derivatives (e.g., MLCs, PLCs or derivatives thereof or lysates thereof or PRP, PRGF, or AS derived therefrom) or the precursor cells for making the megakaryocyte derivatives (e.g., MLCs, PLCs, lysates thereof or derivatives thereof) of the present disclosure. In some embodiments, megakaryocyte derivatives (e.g., MLCs or PLCs or derivatives thereof or lysates thereof or PRP, PRGF, or AS derived therefrom) or precursor cells for making the megakaryocyte derivatives (e.g., MLCs, PLCs, lysates thereof or derivatives thereof or PRP, PRGF, or AS derived therefrom) (i.e., sources for the PRP, PRGF, or AS) is applied first, followed by an inductive cell, or the PRP, PRGF, or AS or the source thereof and the inductive cell are applied simultaneously, or the inductive cell is applied first followed by PRP, PRGF, or AS or the PRP, PRGF, or AS source application. Except when applied simultaneously, one component may be applied within few hours, few days or few weeks of the other depending on the desired application needs of a subject.

In some embodiments, the megakaryocyte derivatives (e.g., MLCs, PLCs, derivatives thereof or lysates thereof) advantageously comprises one or more biomarkers selected from matrix metalloproteinase 1 (MMP-1), matrix metalloproteinase 2 (MMP-2), matrix metalloproteinase 3 (MMP-3), matrix metalloproteinase 7 (MMP-7), matrix metalloproteinase 9 (MMP-9), matrix metalloproteinase 10 (MMP-10), matrix metalloproteinase 12 (MMP-12), matrix metalloproteinase 13 (MMP-13), tissue inhibitor matrix metalloproteinase 1 (TIMP-1), tissue inhibitor matrix metalloproteinase 2 (TIMP-2), tissue inhibitor matrix metalloproteinase 3 (TIMP-3), tissue inhibitor matrix metalloproteinase 4 (TIMP-4), transforming growth factor beta-1 (TGF β1), transforming growth factor beta-2 (TGF β2), transforming growth factor beta-3 (TGF β3), angiopoietin-2 (Ang-2), bone morphogenetic protein 9 (BMP-9), epidermal growth factor (EGF), endoglin, endothelin-1, fibroblast growth factors 1 (FGF-1), fibroblast growth factors 2 (FGF-2), follistatin, granulocyte colony stimulating factor (G-CSF), heparin-binding EGF-like growth factor (HB-EGF), hepatocyte growth factor (HGF), interleukin 8 (IL-8), leptin, placental growth factor (PLGF), vascular endothelial growth factor A (VEGF-A), vascular endothelial growth factor C (VEGF-C), vascular endothelial growth factor D (VEGF-D), soluble CD40L (sCD40L), eotaxin, Fms-like tyrosine kinase receptor 3 ligand (FLT-3L), Fractalkine, growth-regulated oncogene α (GROα), interferon alpha 2 (IFN-α2), interferon gamma (IFN-γ), interleukin 1 alpha (IL-1a), interleukin 1 beta (IL-1β), interleukin-1 receptor antagonist (IL-1RA), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 12p40 (IL-12p40), interleukin 12p70 (IL12p70), interleukin 13 (IL-13), interleukin 15 (IL-15), interleukin 17A (IL-17A), interleukin 17E (IL-17E (or IL-25)), interleukin 17F (IL-17F), interleukin 18 (IL-18), interleukin 22 (IL-22), interleukin 27 (IL-27), interferon gamma-induced protein 10 (IP-10), monocyte chemoattractant protein-1 (MCP-1), monocyte chemoattractant protein-3 (MCP-3), macrophage-derived chemokine (MDC), monokine induced by gamma (MIG (or CXCL9)), macrophage inflammatory protein-1 alpha (MIP-1a), macrophage inflammatory protein-1 beta (MIP-10), platelet-derived growth factor AA (PDGF-AA), platelet-derived growth factor AB (PDGF-AB), regulated upon activation, normal T cell expressed and secreted (RANTES), transforming growth factor alpha (TGFα), tumor necrosis factor α (TNFα), tumor necrosis factor β (TNFβ), vascular endothelial growth factor A1 (VEGF-A1), insulin-like growth factor 1 (IGF-1), insulin-like growth factor 2 (IGF-2), angiopoietin-1, a proliferation-inducing ligand (APRIL), and B-cell activating factor (BAFF). In some embodiments, the megakaryocyte derivatives (e.g., MLCs, PLCs, derivatives thereof or lysates thereof) advantageously comprises one or more biomarkers selected from fibroblast growth factor-2 (FGF-2); hepatocyte growth factor (HGF); insulin-like growth factor 1 (IGF-1), regulated upon activation, normal T cell expressed and secreted (RANTES); nerve growth factor (NGF); vascular endothelial growth factor (VEGF-A); vascular endothelial growth factor (VEGF-C); epidermal growth factor (EGF); transforming growth factor-β1 (TGF-β1); transforming growth factor-β2 (TGF-β1); platelet-derived growth factor-AA (PDGF-AA); platelet-derived growth factor-BB (PDGF-BB); platelet-derived growth factor-AA/BB (PDGF-AA/BB); interleukin 2 (IL-2); interleukin 4 (IL-4); interleukin 12p40 (IL-12p40); interleukin 12p70 (IL-12p70); tissue inhibitors of metalloproteinases-1 (TIMP-1); tissue inhibitors of metalloproteinases-2 (TIMP-2); and tissue inhibitors of metalloproteinases-3 (TIMP-3). In some embodiments, ratio of biomarker concentration and total protein concentration of the megakaryocyte derivative is advantageously different than ratio of concentration of the same biomarker and total protein concentration of PRGF. For example, in some embodiments, ratio of EGF concentration and total protein concentration of the megakaryocyte derivatives (e.g., MLCs, PLCs, derivatives thereof or lysates thereof) is advantageously higher than ratio of concentration of EGF and total protein concentration of PRGF. In some embodiments, ratio of PDGF-BB concentration and total protein concentration of the megakaryocyte derivatives (e.g., MLCs, PLCs, derivatives thereof or lysates thereof) is advantageously higher than ratio of concentration of PDGF-BB and total protein concentration of PRGF.

Provided herein are compositions comprising megakaryocyte derivatives described herein. In some embodiments, the compositions further comprise one or more additional therapeutic agents. In some embodiments, the one or more additional therapeutic agents comprise a wound healing agent, a tissue regeneration agent, an antiapoptotic agent, an anti-inflammatory agent, a neurotropic agent, an anti-hormonal agent or an immunomodulatory agent or combinations thereof. In some embodiments, the composition is free of red blood cells or hemoglobin content or white blood cells. In some embodiments, the tissue regeneration agent is one or more of (a) a growth factor selected from one or more of transforming growth factors (TGF), fibroblast growth factors (FGF), platelet-derived growth factors (PDGF), epidermal growth factors (EGF), vascular endothelial growth factors (VEGF), insulin-like growth factors (IGF), platelet-derived endothelial growth factors (PDEGF), platelet-derived angiogenesis factors (PDAF), platelet factors 4 (PF-4), hepatocyte growth factors (HGF) or combinations thereof; and (b) a cytokine selected from one or more of IL-1B, IL-5, IL-6, IL-8, IL-10, IL-12, IL-13, IL-17A, IL-23, TNF alpha or combinations thereof. In some embodiments, administration of the composition in a therapeutic amount results in a statistically significant reduction in injury recovery as measured after administration of the composition relative to administration of vehicle only. In some embodiments, administration of the composition in a therapeutic amount results in a statistically significant improvement in one or more of a pain, stiffness and function as measured after administration of the composition relative to administration of vehicle only.

Second therapeutic agents for use with the megakaryocyte derivatives (e.g., MLCs, PLCs or derivatives thereof or lysates thereof or PRP, PRGF, or AS derived therefrom) could also be selected from one or more of nonsteroidal anti-inflammatory drugs (NSAIDs), cox 2 inhibitors, Suitable corticosteroids include 11-alpha, 17-alpha,21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosonecypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone famesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin. Other compounds that may be used as a substitute for or in addition to a corticosteroid in the methods, compositions, and kits of the present disclosure include, without limitation, A-348441 (Karo Bio), adrenal cortex extract (GlaxoSmithKIine), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495.times.(GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

It may be desirable to administer to the patient other compounds, such as a corticosteroid, tetra-substituted pyrimidopyrimidine, glucocorticoid, beta-catenin protein or polypeptide or agonist thereof, NSAID (e.g., naproxen sodium, diclofenac sodium, diclofenac potassium, aspirin, sulindac, diflunisal, piroxican, indomethacin, ibuprofen, nabunetone, choline magnesium trisalicylate, sodium salicylate, salicylsalicylic acid, fenoprofen, flurbiprofen, ketoprofen, meclofenamate sodium, meloxican, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies of the present disclosure are especially useful for the treatment of immunoinflammatory disorders in combination with other agents—either biologics or small molecules—that modulate the immune response to positively affect disease. Such agents include those that deplete key inflammatory cells, influence cell adhesion, or influence cytokines involved in immune response. This last category includes both agents that mimic or increase the action of anti-inflammatory cytokines such as IL-10, as well as agents inhibit the activity of pro-inflammatory cytokines such as IL-1B, IL-5, IL-6, IL-8, IL-10, IL-12, IL-13, IL-17A, IL-23, MIG, MCP-1, IP-10, or TNF alpha. Agents that inhibit TNF alpha include etanercept, adelimumab, infliximab, and CDP-870. In this example (that of agents blocking the effect of TNF alpha), the combination therapy reduces the production of cytokines, etanercept or infliximab act on the remaining fraction of inflammatory cytokines, providing enhanced treatment. Small molecule immunomodulators include, e.g., p38 MAP kinase inhibitors such as VX 702, SCIO 469, doramapimod, RO 30201195, SCIO 323, TACE inhibitors such as DPC 333, ICE inhibitors such as pranalcasan, and IMPDH inhibitors such as mycophenolate and merimepodib.

Other therapeutic agents that can be administered, prior to, at the same time or after a gap, with the megakaryocyte derivatives (e.g., MLCs or PLCs or derivatives thereof or lysates thereof or PRP, PRGF, or AS derived therefrom) include, thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL™); beta-lapachone; lapachol; colchicines; betulinic acid; a camptothecin (including the synthetic analogue topotecan (HYCAMTIN™), CPT-11 (irinotecan, CAMPTOSAR™), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammal I and calicheamicin omegall; CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN™, morpholino-doxorubicin, cyanomorpholino-doxorubicin, 2-pyrrolino-doxorubicin, doxorubicin HCl liposome injection (DOXIL™), liposomal doxorubicin TLC D-99 (MYOCET™), peglylated liposomal doxorubicin (CAELYX™), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR™), tegafur (UFTORAL™), capecitabine (XELODA™), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE™, FILDESIN™); dacarbazine; mannomustine; mitobronitol; mitolactol; pipobroman; gacytosine; arabinoside (“Ara-C”); thiotepa; taxoid, e.g., paclitaxel (TAXOL™, Bristol-Myers Squibb Oncology, Princeton, N.J.), albumin-engineered nanoparticle formulation of paclitaxel (ABRAXANE™), and docetaxel (TAXOTERE™, Rhome-Poulene Rorer, Antony, France); chloranbucil; 6-thioguanine; mercaptopurine; methotrexate; platinum agents such as cisplatin, oxaliplatin (e.g., ELOXATIN™), and carboplatin; vincas, which prevent tubulin polymerization from forming microtubules, including vinblastine (VELBAN™), vincristine (ONCOVIN™), vindesine (ELDISINE™, FILDESIN™), and vinorelbine (NAVELBINE™); etoposide (VP-16); ifosfamide; mitoxantrone; leucovorin; novantrone; edatrexate; daunomycin; aminopterin; ibandronate; topoisomerase inhibitor RFS 2000; difluoromethylomithine (DMFO); retinoids such as retinoic acid, including bexarotene (TARGRETIN™); bisphosphonates such as clodronate (for example, BONEFOS™ or OSTAC™), etidronate (DIDROCAL™), NE-58095, zoledronic acid/zoledronate (ZOMETA™), alendronate (FOSAMAX™), pamidronate (AREDIA™), tiludronate (SKELID™), or risedronate (ACTONEL™); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE™ vaccine and gene therapy vaccines, for example, ALLOVECTIN™ vaccine, LEUVECTIN™ vaccine, and VAXID™ vaccine; topoisomerase 1 inhibitor (e.g., LURTOTECAN™); rmRH (e.g., ABARELIX™); BAY439006 (sorafenib; Bayer); SU-11248 (sunitinib, SUTENT™, Pfizer); perifosine, COX-2 inhibitor (e.g., celecoxib or etoricoxib), proteosome inhibitor (e.g., PS341); bortezomib (VELCADE™); CCI-779; tipifamib (R11577); orafenib, ABT510; Bcl-2 inhibitor such as oblimersen sodium (GENASENSE™); pixantrone; EGFR inhibitors; tyrosine kinase inhibitors; serine-threonine kinase inhibitors such as rapamycin (sirolimus, RAPAMUNE™); famesyltransferase inhibitors such as lonafamib (SCH 6636, SARASAR); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above.

Therapeutic agents as defined herein also include “anti-hormonal agents” or “endocrine therapeutics” which act to regulate, reduce, block, or inhibit the effects of hormones that can promote the growth of cancer. They may be hormones themselves, including, but not limited to: anti-estrogens and selective estrogen receptor modulators (SERMs), including, for example, tamoxifen (including NOLVADEX™ tamoxifen), raloxifene, droloxifene, 4-hydroxytamoxifen, trioxifene, keoxifene, LY117018, onapristone, toremifene; aromatase inhibitors that inhibit the enzyme aromatase, which regulates estrogen production in the adrenal glands, such as, for example, 4(5)-imidazoles, aminoglutethimide, MEGASE™ megestrol acetate, AROMASIN™ exemestane, formestanie, fadrozole, RIVISOR™ vorozole, FEMARA™ letrozole, and ARIMIDEX™ anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME™ ribozyme) and a HER2 expression inhibitor; vaccines such as gene therapy vaccines, for example, ALLOVECTIN™ vaccine, LEUVECTIN™ vaccine, and VAXID™ vaccine; PROLEUKIN™ rlL-2; LURTOTECAN™ topoisomerase 1 inhibitor; ABARELIX™ rmRH; Vinorelbine and Esperamicins, and pharmaceutically acceptable salts, acids or derivatives of any of the above; as well as combinations of two or more of the above.

### Immunomodulatory Drug

In some embodiments, the present disclosure also encompasses immunomodulatory drugs for use with the megakaryocyte derivatives (e.g., MLCs, PLCs, derivatives thereof or lysates thereof) or precursor cells for making the megakaryocyte derivatives (e.g., MLCs, PLCs, derivatives thereof or lysates thereof or PRP, PRGF, or AS derived therefrom) of the present disclosure. The term “immunomodulatory drug” refers to a class of drugs that modifies the immune system response or the functioning of the immune system, such as by the stimulation of antibody formation and/or the inhibition of peripheral blood cell activity, and include, but are not limited to, thalidomide (a-N-phthalimido-glutarimide) and its analogues, REVLIMID™ (lenalidomide), ACTI-MID™ (pomalidomide), OTEZLA™ (apremilast), and pharmaceutically acceptable salts or acids thereof.

Therapy, according to the present disclosure, may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment. Optionally, a person having a greater risk of developing an inflammatory disease (e.g., a person who is undergoing age-related hormonal changes) may receive treatment to inhibit or delay the onset of symptoms.

In combination therapy, the dosage and frequency of administration of each component of the combination can be controlled independently. For example, one compound may be administered once, twice, or three times per day, or week or month, while the second compound may be administered once per day, week or month. Combination therapy may be given in on-and-off cycles that include rest periods so that the patient's body has a chance to recover from any as yet unforeseen side effects. The compounds may also be formulated together such that one administration delivers both compounds. Except when administered simultaneously, one component in the combination therapy may be administered within few hours, few days or few weeks of the other depending on the desired administration needs of a subject.

In some embodiments, less than 10⁶ PLCs or about 10⁶ to 10⁷ PLCs or about 10⁷ to 10⁸ PLC or about 10′ to 10⁹ PLCs or about 10⁹ to 10¹⁰ PLCs or greater than 10¹⁰ PLCs are administered to the subject. For example it could be about (1, 2, 3, 4, 5, 6, 7, 8, or 9)×10⁶ to (1, 2, 3, 4, 5, 6, 7, 8, or 9)×10⁷ PLCs, about (1, 2, 3, 4, 5, 6, 7, 8, or 9)×10⁷ PLCs to (1, 2, 3, 4, 5, 6, 7, 8, or 9)×10⁸ PLCs, or about (1, 2, 3, 4, 5, 6, 7, 8, or 9)×10⁸ PLCs to (1, 2, 3, 4, 5, 6, 7, 8, or 9)×10⁹ PLCs or about (1, 2, 3, 4, 5, 6, 7, 8, or 9)×10⁹ PLCs to (1, 2, 3, 4, 5, 6, 7, 8, or 9)×10¹⁰ PLCs may be administered to the subject.

In some embodiments, less than 10⁶ MLCs or about 10⁶ to 10⁷ MLCs or about 10⁷ to 10⁸ MLC or about 10⁸ to 10⁹ MLCs or about 10⁹ to 10¹⁰ MLCs or greater than 10¹⁰ MLCs are administered to the subject. For example it could be about (1, 2, 3, 4, 5, 6, 7, 8, or 9)×10⁶ to (1, 2, 3, 4, 5, 6, 7, 8, or 9)×10⁷ MLCs, about (1, 2, 3, 4, 5, 6, 7, 8, or 9)×10⁷ MLCs to (1, 2, 3, 4, 5, 6, 7, 8, or 9)×10⁸ MLCs, or about (1, 2, 3, 4, 5, 6, 7, 8, or 9)×10⁸ MLCs to (1, 2, 3, 4, 5, 6, 7, 8, or 9)×10⁹ MLCs or about (1, 2, 3, 4, 5, 6, 7, 8, or 9)×10⁹ MLCs to (1, 2, 3, 4, 5, 6, 7, 8, or 9)×10¹⁰ MLCs may be administered to the subject.

The present disclosure provides a kit comprising the megakaryocyte derivatives (e.g., MLCs, PLCs or derivatives thereof or lysates thereof or PRP, PRGF, or AS derived therefrom) (i.e., lysates derived from PLCs or derivatives thereof) or precursor cells for making the megakaryocyte derivatives (e.g., MLCs, PLCs or derivatives thereof or PRP, PRGF, or AS derived therefrom) for use according to the present disclosure, and optionally carriers, buffers, emulsifiers or excipients, the kit further comprising instructions for administration to a subject. The kit may optionally contain additionally one or more drugs in one or more containers. The kit may further comprise a label or package insert. The package insert will provide instructions for use, dosage indications, administration, contraindications and/or warnings concerning the use of such contents of the kit. containers may include vials, bottles, syringes, blister pack, and the like. The active agents (e.g., MLCs or PLCs or derivatives thereof or lysates thereof or combinations thereof or any additional drug contained in the kit) ordinarily can be stored as a solid composition, a lyophilized formulation or as an aqueous solution.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### Manufacturing of Megakaryocyte Derivatives

Described herein are methods of making megakaryocyte derivatives. In some embodiments, the methods comprise: (a) an ex-vivo culturing of a population of progenitor cells for a duration to differentiate the progenitor cells to mature megakaryocytes; (b) isolating a population of MLCs, PLCs or derivatives thereof from the megakaryocytes; and (c) concentrating the MLCs, PLCs or derivatives thereof. In some embodiments, the methods further comprise lysing the MLCs, PLCs or derivatives thereof. In some embodiments, the methods further comprise concentrating the lysed MLCs, PLCs or derivatives thereof. In some embodiments, the methods further comprise mixing with a PRP derived from a donor. In some embodiments, the progenitor cells are selected from one or more of human induced pluripotent stem cells (iPSCs), embryonic stem cells (ESCs), mesenchymal stem/stromal cells (MSCs), hematopoietic stem cells, immortalized megakaryocytic progenitor cells, CD34⁺ umbilical cord blood stem cells (UCB cells), CD34⁺ mobilized peripheral blood cells (MPB cells) or CD34+ bone marrow cells. In some embodiments, the ex-vivo culturing of a population of progenitor cells is performed in a bioreactor. In some embodiments, the MLCs, the PLCs or the derivative thereof are isolated from megakaryocytes through a pressure gradient. In some embodiments, the pressure gradient is generated in a bioreactor. In some embodiments, the megakaryocyte derivates, the PLCs, the MLCs, the MLC lysates, and/or derivatives thereof are made using any of the methods described in U.S. Pat. Nos. 11,400,118 and 10,426,799, and/or U.S. patent application Ser. No. 16/957,918, the entire contents of which are incorporated herein by their entirety.

Alternatively, in some embodiments, the methods comprise: (a) an ex-vivo culturing of a population of somatic cells, progenitor cells, stem cells or combinations thereof for a duration to differentiate to mature megakaryocytes; (b) isolating a population of MLCs, PLCs or derivatives thereof from the megakaryocytes through a pressure gradient in the bioreactor; and (c) concentrating the MLCs, PLCs or derivatives thereof. In some embodiments, the methods further comprise lysing the MLCs, PLCs or derivatives thereof. In some embodiments, the methods further comprise concentrating the lysed MLCs, PLCs or derivatives thereof. In some embodiments, the methods further comprise mixing with a PRP derived from a donor. In some embodiments, the progenitor cells are selected from one or more of human induced pluripotent stem cells (iPSCs), hematopoietic stem cells, embryonic stem cells (ESCs), immortalized megakaryocytic progenitor cells, CD34⁺ umbilical cord blood stem cells (UCB cells), CD34⁺ mobilized peripheral blood cells (MPB cells) or CD34+ bone marrow cells. In some embodiments, the ex-vivo culturing of a population of progenitor cells is performed in a bioreactor. In some embodiments, the MLCs, the PLCs or the derivative thereof are isolated from megakaryocytes through a pressure gradient. In some embodiments, the pressure gradient is generated in a bioreactor.

### Exemplary Embodiments

The methods and compositions of the present disclosure advantageously utilize stem cell derived megakaryocyte derivatives (e.g., megakaryocyte-like cell (MLC) derivatives such as the novel anucleated platelets or platelet-like cells or platelet variants (collectively referred to as “PLCs” (or in its singular form: “PLC”)) or derivatives thereof or lysates thereof or platelet rich plasma derived therefrom) to meet unmet needs of treating, repairing or ameliorating diseases, disorders, or injuries related to dry eye for which no adequate or consistent treatments are available by conventional means.

Advantageously, the megakaryocyte derivatives (e.g., MLCs, PLCs, lysates thereof or derivatives thereof) are consistent in quality and composition and can be made at a relatively lower cost, as compared to that obtained from blood from a human donor. More importantly, it eliminates the need of puncturing a patient to withdraw blood. Moreover, the megakaryocyte derivatives (e.g., MLCs or PLCs) are easy to prepare as they come out of a bioreactor or a fluidic device and can be administered in a less aggressive manner than other therapeutic options (e.g., no surgeries or complex medical procedures are involved). The megakaryocyte derivatives (e.g., MLCs, PLCs, lysates thereof or derivatives thereof), produced by the methods of the present disclosure, can be easily scaled up, can be continuous in supply (unhindered by batch to batch variability), are relatively free of contaminants, can be used as supplement a donor derived PRP, PRGF, or AS, can be locally administered at an injury site or in the vicinity thereof, to treat, repair or alleviate dry eye diseases.

Thus, in some embodiments there are provided compositions comprising the megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or PRP, PRGF, or AS derived therefrom) that can be administered directly or near the site of the eye. In some embodiments, the composition comprising the megakaryocyte derivatives (e.g., MLCs, PLCs, lysates thereof or derivatives thereof or PRP, PRGF, or AS derived therefrom) or precursor cells for making the megakaryocyte derivatives (e.g., MLCs, PLCs, lysates thereof or derivatives thereof or PRP, PRGF, or AS derived therefrom) are administered locally or topically in, around the vicinity of, and under the eye. In some embodiments, megakaryocyte derivatives (e.g., MLCs, PLCs, lysates thereof or derivatives thereof) or precursor cells for making the megakaryocyte derivatives (e.g., MLCs, PLCs or derivatives thereof or PRP, PRGF, or AS derived therefrom) are in a lysate form i.e., the megakaryocyte derivatives (e.g., MLCs, PLCs or derivatives thereof) or precursor cells for making the megakaryocyte derivatives (e.g., MLCs, PLCs or derivatives thereof or PRP, PRGF, or AS derived therefrom) are concentrated, and lysates are prepared from the concentrates. In some embodiments, the PLCs or the lysates are concentrated and are diluted prior to use in a carrier, a diluent, or a buffer, or a donor-derived PRP, PRGF, or AS as disclosed herein.

In some embodiments, megakaryocyte derivatives (e.g., MLCs, PLCs or derivatives thereof or lysates thereof) are enriched with growth factors or with agents that stimulate the release of growth factors from the megakaryocyte derivatives (e.g., MLCs, PLCs, lysates thereof or derivatives thereof) or are mixed with the platelet-rich plasma from a subject in a combination that best suits the subject.

The megakaryocyte derivatives (e.g., MLCs, PLCs, lysates thereof or derivatives thereof) are generated by one or more tools and technologies, such as, bioreactors or fluidic devices. Bioreactors or fluidic devices could include, but is not limited to, shear stress, mechanical strain and pulsed electromagnetic field bioreactors, large-scale stirred tank bioreactors, automated bioreactors, rotating wall bioreactors (RWBs), and rocking motions as seen with wave bioreactors, organ-on-chip bioreactors. Other bioreactor configurations that enable continuous, perfusion operation such as packed bed bioreactors (PBBs), fluidized bed bioreactors (FBBs), or PBBs or FBBs including the use of microcarriers, CultiBag bioreactors, and membrane bioreactors such as hollow fiber bioreactors (HFBs) are also contemplated for generating the PLCs/EVs or derivatives thereof of the present disclosure. Operation of the bioreactors may require coupling with an internal or external cell retention device on a recycle line, by centrifugation, sedimentation, ultrasonic separation or microfiltration with spin-filters, alternating tangential flow (ATF) filtration or tangential flow filtration (TFF) or in vivo bioreactors, which are a pocket within the body into which biomaterials (e.g., PLCs or their derivatives or the progenitor cells form which they are derived from) are implanted at a site in need thereof and incubated for an extended period of time. Within these pockets (for example, eyes, glands), the grafts harness the regenerative capacity of the body to recover from a disease or an injury. Non-limiting examples of bioreactors are described, for example, in the co-filed application titled: Simultaneous Welding of Three Components To Form a Bioreactor or Filter Structure (U.S. Application No. 62/981,373) or elsewhere, for example tools and technologies (e.g., bioreactors or fluidic devices) disclosed in U.S. Pat. Nos. 9,795,965; 10,343,163; 9,763,984; 9,993,503; and 10,426,799; US Publication No. 20180334652; PCT Applications PCT/US2018/021354; PCT/US2019/012437, PCT/US19/040021 and U.S. application Ser. No. 16/730,603, each of which is incorporated herein in their entirety by reference. Bioreactors or microfluidic devices known or unknown that can routinely generate MLCs, PLCs or derivatives are also contemplated for use in the present disclosure.

In some embodiments, the source of megakaryocyte derivatives (e.g., MLCs, PLCs or derivatives thereof or lysates derived thereof or PRP, PRGF, or AS derived therefrom) or precursor cells for making the megakaryocyte derivatives (e.g., MLCs, PLCs, lysates thereof or derivatives thereof or PRP, PRGF, or AS derived therefrom) are autologous, i.e., that they are produced utilizing, for example, the CD34+progenitor cells of an individual in need of the megakaryocyte derivative based treatment (e.g., MLC-based treatment, PLC-based treatment), wherein the progenitor cells are cultured through a bioreactor to produce the megakaryocyte derivatives (e.g., MLCs, PLCs, lysates thereof or derivatives thereof), which can be used as such, i.e., as megakaryocyte derivatives (e.g., MLCs, PLCs or derivatives thereof or lysates thereof or PRP, PRGF, or AS derived therefrom (i.e., PRP, PRGF, or AS enriched with the MLCs, PLCs, lysates thereof or derivatives thereof)). In some embodiments, the source of megakaryocyte derivatives (e.g., MLCs, PLCs or derivatives thereof or lysates thereof or PRP, PRGF, or AS derived therefrom) are iPSCs or the megakaryocytes derived from the iPSCs.

Several other advantages provided by the methods and compositions comprising megakaryocyte derivatives (e.g., MLCs, PLCs or derivatives thereof or lysates thereof or PRP, PRGF, or AS derived therefrom) are that they are essentially allogeneic, which minimizes the risk of immune responses, are not cancerous, i.e., do not exhibit uncontrolled growth or tumor formation in vivo, and are enriched in growth factors, such as but not limited to, fibronectin, vitronectin, sphingosine 1-phosphate, which facilitate the healing process. megakaryocyte derivatives (e.g., MLCs or PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom) may also reduce or eliminate detrimental side-effects elicited by anti-inflammatory agents, opioids, or other drugs, for example.

In some embodiments, the present disclosure provides a method of treating a subject suffering from an injury, the method comprising administering to the subject in therapeutic amounts a composition comprising megakaryocyte derivatives (e.g., MLCs, PLCs or derivatives thereof or lysates thereof or the PRP, PRGF, or AS derived therefrom) of the present disclosure thereby causing amelioration of or treatment or repair of the injuries (e.g., dry eye diseases). In some embodiments, the method comprises administering a second or a third therapeutic agent.

In some embodiments, the present disclosure provides a pharmaceutical composition comprising megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs or derivatives thereof or lysates thereof or PRP, PRGF, or AS derived therefrom) and one or more pharmaceutically acceptable bulking agent, a carrier or excipient. In some embodiments, the pharmaceutical composition further comprises a second or a third therapeutic agent.

In some embodiments, the present disclosure provides non-natural extracellular vesicles (EVs) that are made in vitro as admixtures with the megakaryocyte derivatives (e.g., MLCs, PLCs, lysates thereof or derivatives thereof). EVs comprise microvesicles (MV) and exosomes, 200-1000 nm in diameter, carrying biologically active multifarious molecules such as proteins, lipids, and RNAs either on their surface or within their lumen. Each component in the admixture, i.e., megakaryocyte derivatives (e.g., MLCs, PLCs, lysates thereof, derivatives thereof), microvesicles and exosomes can substantially be isolated into individual components from the admixture, for example based on their size. The extracellular vesicles (EVs) are implicated to play a role in stimulating eye healings, indicating that they can confer, for example, eye healing, antiapoptotic or anti-inflammatory actions through transporting RNA and protein cargos. They also function as a transport and delivery system for bioactive molecules, play a role in hemostasis and thrombosis, inflammation, malignancy infection transfer, angiogenesis, and immunity. Thus, in some embodiments, EVs may complement megakaryocyte derivatives (e.g., MLCs, PLCs, lysates thereof or their derivatives) and their combinational use is an even richer resource for megakaryocyte derivative based therapeutic applications (e.g., MLC-based therapeutic applications, PLC-based therapeutic applications).

In some embodiments, the EVs of the present disclosure comprise exosomes, approximately ranging between 65 nm to about 10 μm in diameter carrying multifarious molecules such as proteins, lipids, and RNAs either on their surface or within their lumen. Exosomes play a role in stimulating tissue regeneration, in many in vitro and in vivo models, demonstrating that they can confer proangiogenic, proliferative, antiapoptotic and anti-inflammatory actions through transporting RNA and protein cargos. Thus, in some embodiments, exosomes make it even a richer resource for megakaryocyte derivative based therapeutic applications (e.g., MLC-based therapeutic applications, PLC-based therapeutic applications). In some embodiments, megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs, lysates thereof or derivatives thereof or PRP, PRGF, or AS derived therefrom) are administered in combination with the extracellular vesicles (EVs) that are produced as admixtures with the megakaryocyte derivatives (e.g., MLCs, PLCs, lysates thereof or derivatives thereof) but can substantially be isolated from the megakaryocyte derivatives (e.g., MLCs, PLCs) based on their smaller size. Thus, in an aspect, the microvesicles or the exosomes, alone or in combination with megakaryocyte derivatives (e.g., MLCs, PLCs, lysates thereof or derivatives thereof), can be used for stimulating healing dry eye diseases, for example, through actions such as transporting RNA or transporting agents (e.g., eye healing agents, antiapoptotic agents, anti-inflammatory agents or the like) or protein cargos, thereby making them even a richer resource for megakaryocyte derivative based therapeutic applications (e.g., MLC-based therapeutic applications, PLC-based therapeutic applications) to confer proangiogenic, proliferative, antiapoptotic or anti-inflammatory effects that may be desired in a patient undergoing PLC-based treatment as disclosed herein.

In some embodiments, the megakaryocytic progenitors, megakaryocytes, proplatelets, preplatelets derived from induced pluripotent stem cells (iPSCs), which produce the megakaryocyte-like cells (MLCs), platelet like cells (PLCs) and EVs (i.e., microvesicles or exosomes or a combination thereof), prior to passaging through a bioreactor or a fluidic device, can be genetically engineered to express a nucleic acid encoding a protein of interest (for example, eye healing agents, antiapoptotic agents, anti-inflammatory agents or the like). In some embodiments, MLC, PLC and/or EVs can be genetically engineered once such cells were subjected to a passage through the bioreactor or a fluidic device. Thus, in some embodiments, genetic modifications can take place at the stem cell level, in megakaryocytes or in some embodiments in the PLCs and/or the EVs or at any other level during the generation of MLCs, PLCs and/or the EVs that accompany the MLC, PLC and/or EV production. Genetic engineering of megakaryocytes or megakaryocytic progenitors differentiated from a genetically engineered human pluripotent stem cells (hPSCs) cell or cell lines, where the genetic manipulation leads to megakaryocytes or megakaryocytic progenitor cells to express a protein or a polypeptide of interest are also contemplated by the present disclosure. In some embodiments, the MLCs, PLCs and/or EVs or derivatives thereof, differentiated from the genetically engineered progenitor cells (e.g., megakaryocytes or megakaryocytic progenitor cells), deliver a protein of interest (e.g., eye healing agents, antiapoptotic agents, anti-inflammatory agents or the like) systemically or at first diseased location, generally the site of a disease where the MLCs, PLCs and/or the EVs (or genetically engineered versions thereof) are administered, or to a second diseased location, different from the site where the MLCs, PLCs and/or EVs or derivatives thereof are administered. Examples of such genetically engineered induced pluripotent stem cells or PSC-derived megakaryocytes that produce the PLCs and/or EVs (i.e., genetically engineered PLCs/EVs or derivatives thereof) are disclosed in co-pending U.S. patent application Ser. Nos. 17/213,552 and 17/213,796, respectively, incorporated herein in their entireties by reference. Thus, in some embodiments, genetically engineered PLCs (ePLCs) may be produced by genetically engineered PLC-producing progenitor cells such that ePLCs express an exogenous gene of interest for example for eye healing agents, antiapoptotic agents, anti-inflammatory agents, anti-hormonal agents or immunomodulatory agents or the like, enrichment of which will broadly complement the PLC-based treatments.

It may be desirable to administer to the patient other compounds, such as but not limited to a corticosteroid, tetra-substituted pyrimidopyrimidine, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies of the present disclosure are especially useful for the treatment of immunoinflammatory disorders in combination with other agents—either biologics or small molecules—that modulate the immune response to positively affect disease treatments. Such agents include those that deplete key inflammatory cells, influence cell adhesion, or influence cytokines involved in immune response. Ibis last category includes both agents that mimic or increase the action of anti-inflammatory cytokines such as IL-10, as well as agents inhibit the activity of pro-inflammatory cytokines such as IL-1B, IL-5, IL-6, IL-8, IL-10, IL-12, IL-13, IL-17A, IL-23, MIG, MCP-1, IP-10 or TNF alpha. Agents that inhibit TNF alpha include etanercept, adelimumab, infliximab, and CDP-870. In this example (that of agents blocking the effect of TNF alpha), the combination therapy reduces the production of cytokines, etanercept or infliximab act on the remaining fraction of inflammatory cytokines, providing enhanced treatment. Small molecule immunomodulators include, e.g., p38 MAP kinase inhibitors such as VX 702, SCIO 469, doramapimod, RO 30201195, SCIO 323, TACE inhibitors such as DPC 333, ICE inhibitors such as pranalcasan, and IMPDH inhibitors such as mycophenolate and merimepodib. Advantageously, when one or more of these compounds are administered with megakaryocyte derivatives (e.g., MLCs, PLCs, lysates thereof or derivatives therefrom), it is expected that the concentration of doses of co-administered compounds, if included in a co-therapy, will be reduced or lowered, thereby reducing any harmful known and unknown side-effect induced by the co-administered compounds if they were administered without the megakaryocyte derivatives (e.g., MLCs, PLCs, lysates thereof or derivatives thereof).

In some embodiments, the present disclosure provides kit comprising megakaryocyte derivatives (e.g., MLCs, PLCs or derivatives thereof or lysates thereof or PRP, PRGF, or AS derived therefrom) or precursor cells for making the megakaryocyte derivatives (e.g., MLCs, PLCs, lysates thereof or derivatives thereof or PRP, PRGF, or AS derived therefrom) of the present disclosure.

In some aspects, the techniques described herein relate to a method of treating, repairing or ameliorating a condition in a subject in need of such treatment including administering to the subject an effective amount of a treating composition including megakaryocyte derivatives (e.g., megakaryocyte-like cells (MLCs), platelet-like cells (PLCs) or derivatives thereof or lysates or PRP, PRGF, or AS derived therefrom), wherein the condition is a dry eye disease, wherein an administration route is ophthalmic or intraglandular.

In some aspects, the techniques described herein relate to a method for producing megakaryocyte derivatives (e.g., MLC or PLC) including PRP, the method including: an ex-vivo culturing in a bioreactor a population of progenitor cells for a duration to differentiate the progenitor cells to mature megakaryocytes; isolating a population of MLCs, PLCs or derivatives thereof or lysates or PRP, PRGF, or AS derived therefrom subjected to a separation from the megakaryocytes through a pressure gradient in the bioreactor; concentrating the MLCs, PLCs or derivatives thereof or lysates or PRP, PRGF, or AS derived therefrom; optionally lysing the MLCs, PLCs or derivatives thereof or lysates or PRP, PRGF, or AS derived therefrom; and, optionally mixing with a PRP derived from a donor.

In some aspects, the techniques described herein relate to a method of treating a dry eye disease in a patient with megakaryocyte derivatives (e.g., an MLC derivative, such as MLC lysate, an in vitro anucleated population of platelet like cell (PLCs) or derivatives thereof or lysates or PRP, PRGF, or AS derived therefrom) possessing the following characteristics: i) is derived from reprogramming of a somatic cell, progenitor cell or stem cell, products of which passage through a bioreactor ii) is not a cancerous cell; iii) does not exhibit uncontrolled growth or tumor formation in vivo, wherein the megakaryocyte derivatives (e.g., MLCs, PLCs or derivatives thereof or lysates or PRP, PRGF, or AS derived therefrom) are administered in a therapeutic amount in the patient in need of such treatment, wherein an administration route is ophthalmic, intraglandular, subconjunctival, topical, sub-Tenon's, intravitreal, or intracameral.

In some aspects, the techniques described herein relate to a method of treating a dry eye disease in a subject in need thereof including administering an effective amount of a composition including megakaryocyte derivatives (e.g., MLCs, PLCs or derivatives thereof or lysates or PRP, PRGF, or AS derived therefrom) to the subject, wherein the composition is administered in a therapeutic amount and wherein a statistically significant reduction in injury recovery is measured after administration of the composition relative to administration of vehicle only and/or a statistically significant improvement in one or more of a pain, stiffness and function as measured after administration of the composition relative to administration of vehicle only, wherein an administration route is ophthalmic, intraglandular, subconjunctival, topical, sub-Tenon's, intravitreal, or intracameral.

In some aspects, the techniques described herein relate to a method of treating, repairing or ameliorating a dry eye disease in a subject in need of such treatment including administering to the subject more than one dose of an effective amount of a treating composition including megakaryocyte derivatives (e.g., megakaryocyte-like cells (MLCs), platelet-like cells (PLCs) or derivatives thereof or lysates or PRP, PRGF, or AS derived therefrom), wherein an administration route is ophthalmic or intraglandular.

In some aspects, the techniques described herein relate to a method of treating, repairing or ameliorating a dry eye disease in a subject in need of such treatment including administering to the subject more than one dose of an effective amount of a treating composition including megakaryocyte derivatives (e.g., megakaryocyte-like cells (MLCs), platelet-like cells (PLCs) or derivatives thereof or lysates or PRP, PRGF, or AS derived therefrom), wherein the disease is not cancer or the treatment is not for cancer, and wherein an administration route is ophthalmic, intraglandular, subconjunctival, topical, sub-Tenon's, intravitreal, or intracameral.

In some aspects, the techniques described herein relate to a composition for treating a dry eye disease in a subject, the composition including megakaryocyte derivatives (e.g., MLCs, PLCs or derivatives thereof or lysates or PRP, PRGF, or AS derived therefrom), wherein an administration route is ophthalmic, intraglandular, subconjunctival, topical, sub-Tenon's, intravitreal, or intracameral.

Provided herein are methods of treating, repairing or ameliorating dry eye disease in a subject in need of such treatment, the methods comprise administering to the subject an effective amount of a composition comprising a megakaryocyte derivative, wherein an administration route is ophthalmic or intraglandular, wherein the megakaryocyte derivative includes MLC lysates, PLCs, exosomes, megakaryocytes, or combinations thereof. Further provided herein are methods of treating, repairing or ameliorating dry eye disease in a subject in need of such treatment, wherein the composition further comprises a wound healing agent, a tissue regeneration agent, an antiapoptotic agent, an anti-inflammatory agent, a neurotropic agent, an anti-hormonal agent or an immunomodulatory agent or a combination thereof. Further provided herein are methods of treating, repairing or ameliorating dry eye disease in a subject in need of such treatment, wherein the tissue regeneration agent is one or more of (a) a growth factor selected from one or more of transforming growth factors (TGF), fibroblast growth factors (FGF), platelet-derived growth factors (PDGF), epidermal growth factors (EGF), vascular endothelial growth factors (VEGF), insulin-like growth factors (IGF), platelet-derived endothelial growth factors (PDEGF), platelet-derived angiogenesis factors (PDAF), platelet factors 4 (PF-4), hepatocyte growth factors (HGF) or combinations thereof; and (b) a cytokine selected from one or more of IL-1B, IL-5, IL-6, IL-8, IL-10, IL-12, IL-13, IL-17A, IL-23, TNF alpha or combinations thereof. Further provided herein are methods of treating, repairing or ameliorating dry eye disease in a subject in need of such treatment, wherein the megakaryocyte derivative comprises one or more biomarkers selected from fibroblast growth factor-2 (FGF-2); hepatocyte growth factor (HGF); insulin-like growth factor 1 (IGF-1), regulated upon activation, normal T cell expressed and secreted (RANTES); nerve growth factor (NGF); vascular endothelial growth factor (VEGF-A); vascular endothelial growth factor (VEGF-C); epidermal growth factor (EGF); transforming growth factor-β1 (TGF-β1); transforming growth factor-β2 (TGF-β1); platelet-derived growth factor-AA (PDGF-AA); platelet-derived growth factor-BB (PDGF-BB); platelet-derived growth factor-AA/BB (PDGF-AA/BB); interleukin 2 (IL-2); interleukin 4 (IL-4); interleukin 12p40 (IL-12p40); interleukin 12p70 (IL-12p70); tissue inhibitors of metalloproteinases-1 (TIMP-1); tissue inhibitors of metalloproteinases-2 (TIMP-2); and tissue inhibitors of metalloproteinases-3 (TIMP-3). Further provided herein are methods of treating, repairing or ameliorating dry eye disease in a subject in need of such treatment, wherein the megakaryocyte derivative comprises EGF concentration per unit total protein that is higher than an average EGF concentration per unit total protein measured in human platelets, platelet-rich plasma (PRP), or plasma rich in growth factors (PRGF), or in a range that is higher than corresponding EGF concentration in total protein concentration of PRGF. Further provided herein are methods of treating, repairing or ameliorating dry eye disease in a subject in need of such treatment, wherein the composition further comprises lysates, serum, plasma, plasma rich in growth factors (PRGF), platelet rich plasma (PRP), or any other blood derivative derived from the subject. Further provided herein are methods of treating, repairing or ameliorating dry eye disease in a subject in need of such treatment, wherein the ophthalmic administration route is subconjunctival or topical. Further provided herein are methods of treating, repairing or ameliorating dry eye disease in a subject in need of such treatment, wherein the ophthalmic administration route is sub-Tenon's. Further provided herein are methods of treating, repairing or ameliorating dry eye disease in a subject in need of such treatment, wherein the ophthalmic administration route is intravitreal or intracameral. Further provided herein are methods of treating, repairing or ameliorating dry eye disease in a subject in need of such treatment, wherein the dry eye disease is caused by Sjogren's syndrome or non-Sjogren's syndrome. Further provided herein are methods of treating, repairing or ameliorating dry eye disease in a subject in need of such treatment, wherein the megakaryocyte derivative is free of red blood cells or hemoglobin content or white blood cells. Further provided herein are methods of treating, repairing or ameliorating dry eye disease in a subject in need of such treatment, wherein the composition further comprises extracellular vesicles (EV). Further provided herein are methods of treating, repairing or ameliorating dry eye disease in a subject in need of such treatment, wherein the composition is formulated for application to a site of injury or tissue damage for therapeutic use. Further provided herein are methods of treating, repairing or ameliorating dry eye disease in a subject in need of such treatment, wherein the composition in formulated in a buffer, diluent, or excipient or a combination thereof. Further provided herein are methods of treating, repairing or ameliorating dry eye disease in a subject in need of such treatment, wherein the composition comprises: a) between 0.01 and 100 wt % of the megakaryocyte derivative, b) between 0 and 90 wt. % of a bulking agent, and/or c) between 0 and 90 wt. % of at least one excipient or carrier and optionally d) platelet-rich plasma (PRP), PRGF, plasma, serum, or other blood derivative derived from the subject. Further provided herein are methods of treating, repairing or ameliorating dry eye disease in a subject in need of such treatment, wherein the composition is lyophilized. Further provided herein are methods of treating, repairing or ameliorating dry eye disease in a subject in need of such treatment, wherein the composition is implanted on an implantable device. Further provided herein are methods of treating, repairing or ameliorating dry eye disease in a subject in need of such treatment, wherein the composition is cryopreserved. Further provided herein are methods of treating, repairing or ameliorating dry eye disease in a subject in need of such treatment, wherein the composition is locally administered at one or more of a site of or near an injury or a disease. Further provided herein are methods of treating, repairing or ameliorating dry eye disease in a subject in need of such treatment, wherein the composition further comprises another therapeutic agent. Further provided herein are methods of treating, repairing or ameliorating dry eye disease in a subject in need of such treatment, wherein the megakaryocyte derivative is derived from reprogramming of a somatic cell, progenitor cell or stem cell. Further provided herein are methods of treating, repairing or ameliorating dry eye disease in a subject in need of such treatment, wherein the megakaryocyte derivative is not a cancerous cell. Further provided herein are methods of treating, repairing or ameliorating dry eye disease in a subject in need of such treatment, wherein the megakaryocyte derivative does not exhibit uncontrolled growth or tumor formation in vivo. Further provided herein are methods of treating, repairing or ameliorating dry eye disease in a subject in need of such treatment, wherein the method reduces injury recovery time. Further provided herein are methods of treating, repairing or ameliorating dry eye disease in a subject in need of such treatment, wherein the method improves one or more of pain, stiffness and function of eye, or one or more signs and/or symptoms of dry eye disease, or one or more of pain, discomfort, visual function, ocular surface disease index, corneal fluorescein score, tear production, tear quality, tear break-up-time, immune cell infiltration, goblet cell numbers, mucin levels, etc. Further provided herein are methods of treating, repairing or ameliorating dry eye disease in a subject in need of such treatment, wherein the megakaryocyte derivative is made in a fluidic device or a bioreactor.

Provided herein are methods of treating, repairing or ameliorating a condition in a subject in need of such treatment comprising administering to the subject more than one dose of an effective amount of a composition comprising a megakaryocyte derivative, wherein the condition is selected from dry eye disease, ocular surface disease, osteoarthritis, and wound healing. Further provided herein are methods of treating, repairing or ameliorating a condition in a subject in need of such treatment, wherein the megakaryocyte derivative includes MLC lysates, PLCs, exosomes, megakaryocytes, or combinations thereof. Further provided herein are methods of treating, repairing or ameliorating a condition in a subject in need of such treatment, wherein the megakaryocyte derivative comprises one or more biomarkers selected from fibroblast growth factor-2 (FGF-2); hepatocyte growth factor (HGF); insulin-like growth factor 1 (IGF-1), regulated upon activation, normal T cell expressed and secreted (RANTES); nerve growth factor (NGF); vascular endothelial growth factor (VEGF-A); vascular endothelial growth factor (VEGF-C); epidermal growth factor (EGF); transforming growth factor-β1 (TGF-β1); transforming growth factor-β2 (TGF-β1); platelet-derived growth factor-AA (PDGF-AA); platelet-derived growth factor-BB (PDGF-BB); platelet-derived growth factor-AA/BB (PDGF-AA/BB); interleukin 2 (IL-2); interleukin 4 (IL-4); interleukin 12p40 (IL-12p40); interleukin 12p70 (IL-12p70); tissue inhibitors of metalloproteinases-1 (TIMP-1); tissue inhibitors of metalloproteinases-2 (TIMP-2); and tissue inhibitors of metalloproteinases-3 (TIMP-3). Further provided herein are methods of treating, repairing or ameliorating a condition in a subject in need of such treatment, wherein the condition is selected from dry eye disease, ocular surface disease, osteoarthritis, and wound healing. Further provided herein are methods of treating, repairing or ameliorating a condition in a subject in need of such treatment, wherein the composition further comprises a wound healing agent, a tissue regeneration agent, an antiapoptotic agent, an anti-inflammatory agent, a neurotropic agent, an anti-hormonal agent or an immunomodulatory agent or a combination thereof. Further provided herein are methods of treating, repairing or ameliorating a condition in a subject in need of such treatment, wherein the more than one dose is administered, daily, weekly, biweekly, triweekly or monthly. Further provided herein are methods of treating, repairing or ameliorating a condition in a subject in need of such treatment, wherein the composition is administered by any one of route of administrations selected from topical, transdermal, or systemic route of administration. Further provided herein are methods of treating, repairing or ameliorating a condition in a subject in need of such treatment, wherein the composition is administered by any one of route of administrations selected from subconjunctival, topical, sub-Tenon's, intravitreal, intracameral, intravenous, intraarterial, intramuscular, subcutaneous, inhalation, rectal, buccal, vaginal, intraperitoneal, intraarticular, intraglandular into a lacrimal gland, topical, otic, or oral route of administration. Further provided herein are methods of treating, repairing or ameliorating a condition in a subject in need of such treatment, wherein (a) the conditions is a dry eye disease, (b) the treatment is not for cancer, and (c) an administration route is ophthalmic, intraglandular, subconjunctival, topical, sub-Tenon's, intravitreal, or intracameral. Further provided herein are methods of treating, repairing or ameliorating a condition in a subject in need of such treatment, wherein the composition is diluted to a physiological concentration in a carrier, wherein the carrier comprises a diluent or an excipient. Further provided herein are methods of treating, repairing or ameliorating a condition in a subject in need of such treatment, wherein the carrier is a plasma or a plasma substitute or a plasmalyte or saline. Further provided herein are methods of treating, repairing or ameliorating a condition in a subject in need of such treatment, wherein the megakaryocyte derivative is made in a fluidic device or a bioreactor.

Provided herein are method for producing megakaryocyte derivatives, the methods comprise: an ex-vivo culturing of a population of progenitor cells for a duration to differentiate the progenitor cells to mature megakaryocytes; isolating a population of MLCs, PLCs or derivatives thereof by separating from the megakaryocytes; concentrating the MLCs, PLCs or derivatives thereof; optionally lysing the MLCs, PLCs or derivatives thereof; and optionally mixing with a PRP derived from a donor, wherein the megakaryocyte derivative includes MLC lysates, PLCs, exosomes, megakaryocytes, or combinations thereof. Further provided herein are method for producing megakaryocyte derivatives, wherein the progenitor cells are selected from one or more of human induced pluripotent stem cells (iPSCs), hematopoietic stem cells, embryonic stem cells (ESCs), immortalized megakaryocytic progenitor cells, CD34+ umbilical cord blood stem cells (UCB cells), CD34+ mobilized peripheral blood cells (MPB cells) or CD34+ bone marrow cells. Also provided herein are composition for treating a dry eye disease in a subject, the composition is made according to any one of the methods described herein. Further provided herein are method for producing megakaryocyte derivatives, wherein the megakaryocyte derivative comprises one or more biomarkers selected from fibroblast growth factor-2 (FGF-2); hepatocyte growth factor (HGF); insulin-like growth factor 1 (IGF-1), regulated upon activation, normal T cell expressed and secreted (RANTES); nerve growth factor (NGF); vascular endothelial growth factor (VEGF-A); vascular endothelial growth factor (VEGF-C); epidermal growth factor (EGF); transforming growth factor-β1 (TGF-β1); transforming growth factor-β2 (TGF-β1); platelet-derived growth factor-AA (PDGF-AA); platelet-derived growth factor-BB (PDGF-BB); platelet-derived growth factor-AA/BB (PDGF-AA/BB); interleukin 2 (IL-2); interleukin 4 (IL-4); interleukin 12p40 (IL-12p40); interleukin 12p70 (IL-12p70); tissue inhibitors of metalloproteinases-1 (TIMP-1); tissue inhibitors of metalloproteinases-2 (TIMP-2); and tissue inhibitors of metalloproteinases-3 (TIMP-3). Further provided herein are method for producing megakaryocyte derivatives, wherein the megakaryocyte derivative comprises EGF concentration per unit total protein that is higher than an average EGF concentration per unit total protein measured in human platelets, platelet-rich plasma (PRP), or plasma rich in growth factors (PRGF), or in a range that is higher than corresponding EGF concentration in total protein concentration of PRGF. Further provided herein are method for producing megakaryocyte derivatives, wherein the composition further comprises a wound healing agent, a tissue regeneration agent, an antiapoptotic agent, an anti-inflammatory agent, a neurotropic agent, an anti-hormonal agent or an immunomodulatory agent or a combination thereof. Further provided herein are method for producing megakaryocyte derivatives, wherein the megakaryocyte derivative is free of red blood cells or hemoglobin content or white blood cells. Further provided herein are method for producing megakaryocyte derivatives, wherein the composition further comprises extracellular vesicles (EV). Further provided herein are method for producing megakaryocyte derivatives, wherein the composition is formulated for application to a site of injury or tissue damage for therapeutic use. Further provided herein are method for producing megakaryocyte derivatives, wherein the composition further comprises another therapeutic agent. Further provided herein are method for producing megakaryocyte derivatives, wherein the megakaryocyte derivative is made in a fluidic device or a bioreactor.

### EXAMPLES

### Example 1: Dry Eye Disease (DED) Studies in Mouse DS Model Showing Significant Improvement in DED Conditions

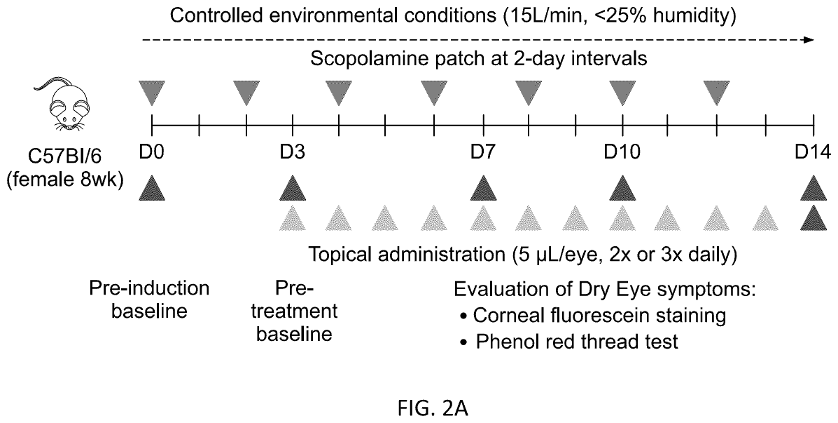

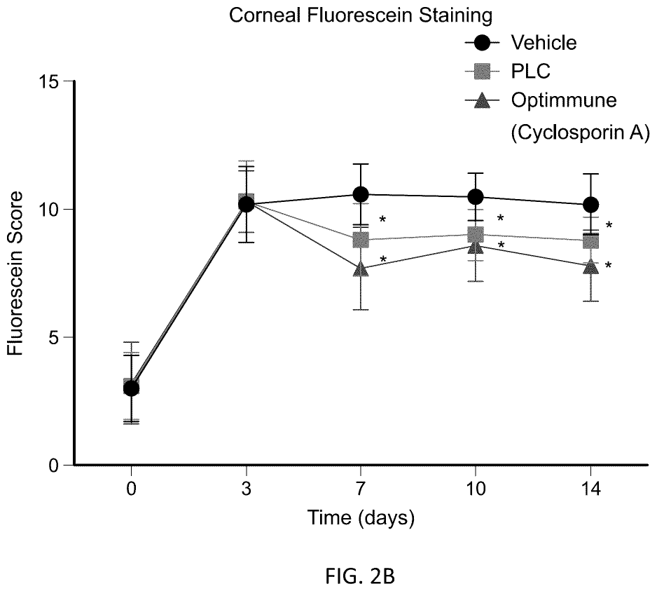

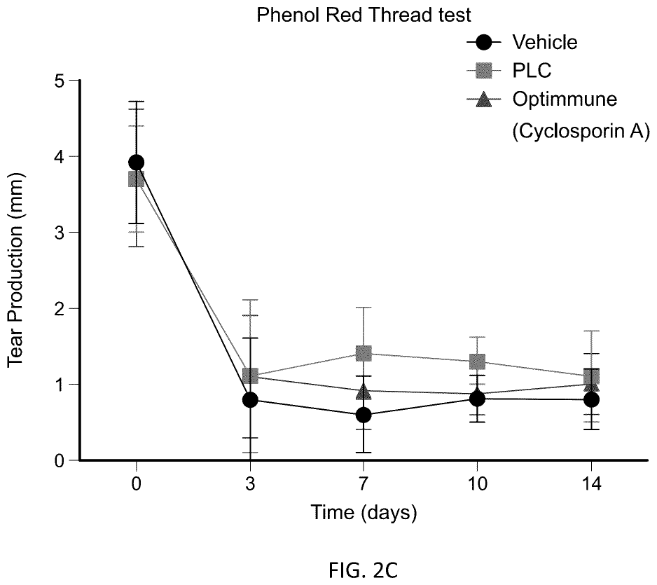

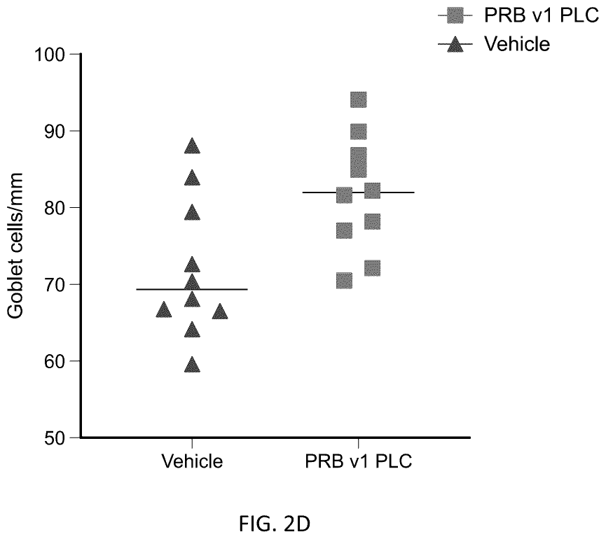

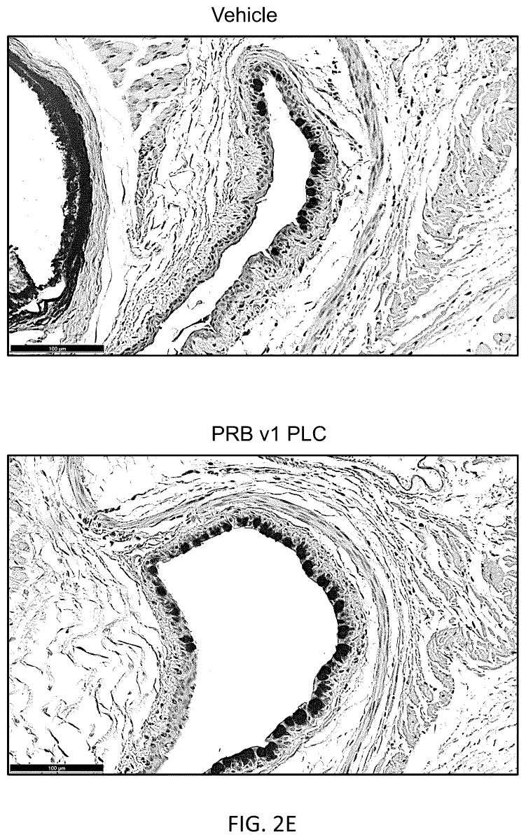

In vivo effects of megakaryocyte derivatives (e.g., PLCs, MLC lysate) in subjects with dry eye disease were measured. Briefly, a mouse desiccative stress (DS)-induced DED model was used in a first in vivo study as shown in FIG. 2A for testing PLCs. The DS model uses slow-release of scopolamine administration, which is an acetylcholine antagonist, to block tear production, in combination with low humidity environmental conditions, to induce DED in mice similar to human DED.

Groups of 30 female C57BI/6 mice aged 8 weeks were evaluated for DED symptoms starting on day 0, then a scopolamine patch was applied, and the mice were placed in a controlled environment (e.g., 15 L/min, <25% humidity). The scopolamine patch was replaced at 2-day intervals over the course of 2 weeks. After 3 days of DED induction, mice were evaluated again for DED symptoms and were then treated by topical administration of 5 μL/eye of either PLC in diluted Form E (n=10, 2× daily administration), a vehicle control (n=10, 2× daily administration), or a 2% Cyclosporin A (CsA) ointment (Optimmune®) (n=10, 3× daily administration). The vehicle was used as a negative control, and the CsA was used as a positive control. Follow-up evaluation of DED symptoms were performed at days 7, 10, and 14. At Day 14, the mice were sacrificed, and the eyeballs were dissected for histological evaluation. Form E is a freezing medium consisting of PlasmaLyte A and 250 mM trehalose. Upon thawing of Form E, it was diluted with sterile water to a final osmolarity of 280-300 mOsm and pH 6.7-7. Optimmune® was used as formulated.

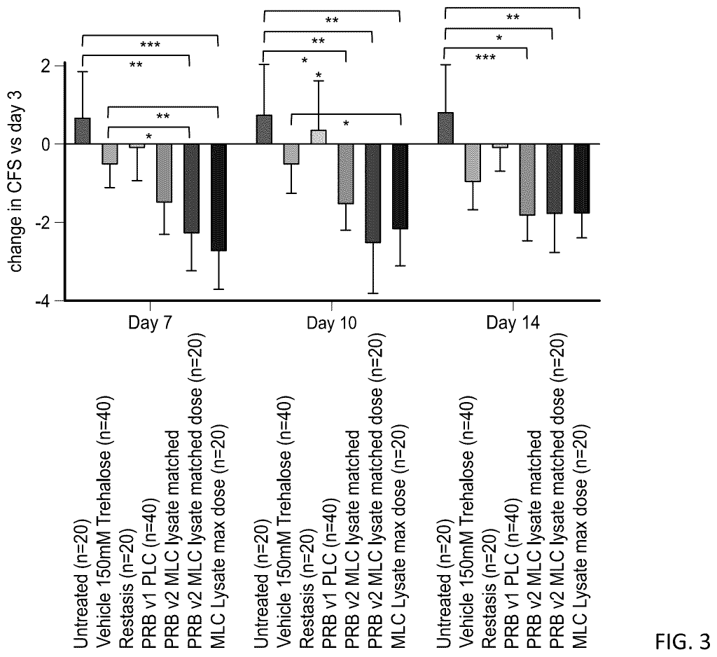

Corneal fluorescein staining and phenol red thread tests were used to assess the status of DED in these animals at days 0, 3, 7, 10 and 14. Corneal fluorescein was used as primary endpoint, the results of which are provided in FIG. 3. Here, corneal defects were examined by slit-lamp observation using blue light after instillation of 0.5 μL of 0.5% sodium fluorescein. Defects are assessed based on a measured degree of damage at the corneal surface, as indicated by staining for punctate keratitis. The scale is based on the National Eye Institute (NEI) grading system, such that the cornea is divided into five areas, and the amount of staining in each area is graded from 0 to 3 according to the punctate fluorescein staining. The maximum score is 15.

TABLE 1
provides the scale for the grading.

| SCORE | SCALE |
|---|---|
| 0 | No staining |
| 1 | Mild punctate staining |
| 2 | Moderate punctate staining |
| 3 | Severe punctate staining |

FIG. 2B provides results from a study using PLCs as a test treatment. CsA was used as a positive control. The * represents statistically significant difference from the vehicle (p<0.05), indicating a significant difference and therefore successful treatment with the PLCs at days 7, 10 and 14 as compared to the vehicle for reducing corneal damage. It should be noted that there is no significant difference between the PLC and CsA at any time point.

Further evaluation was achieved using a phenol red thread test, the results of which are provided in FIG. 2C. Here, tear production was measured with a cotton thread test in the lateral canthus of the conjunctival fornix for 30 seconds. FIG. 2C provides results of treatment with PLCs. CsA was used as a positive control. Again, the * represents statistically significant difference from the vehicle (p<0.05), indicating a significant difference and therefore successful treatment with the PLCs at days 7 and 10 as compared to the vehicle for increasing tear production. It should again be noted that there is no significant difference between the PLC and CsA at any time point.

In a follow-up second study for testing PLCs, mouse DS-induced DED model was used. The second study was performed using 60 female mice. Desiccative stress was induced identically to the first study, and test subjects were administered twice per day from day 3 through day 14. Cyclosporine A was administered in the form of Restasis® eye drops. In addition to the vehicle control, an untreated negative control arm was included. Test groups were administered with PLC and two different doses of MLC Lysate. MLC lysate matched dose and MLC lysate max dose. The MLC lysate matched dose comprised the same total protein content as the PLC dose used in both studies, while the MLC lysate max dose comprised approximately 7.3-fold higher total protein content than the PLC dose. Additional study parameters are as follows. The subjects were divided into six groups, which comprised: Untreated (n=10), Vehicle (n=10), PLC (n=10), MLC Lysate matched dose (n=10), MLC Lysate max dose (n=10), Restasis® 0.05% emulsion (n=10). All formulations were prepared using Form E (PlasmaLyte, 150 mM Trehalose).

FIGS. 2D and 2E illustrate effects of the DS-induced DED model on goblet cells. Goblet cells of the right eye counted in total around temporal, middle, and nasal areas of conjunctiva. Cells averaged per mm of tissue counted from palpebral to bulbar conjunctiva (including the fornix) for all 3 sections per mouse. Mice in vehicle cohort show marked reduction of goblet cells compared to PLC-treated animals (FIG. 2D). Without bound to a specific theory, an analysis of FIG. 2D suggests that amelioration of goblet cell loss can be a mechanism of action for the positive effects of PLC observed in the desiccative stress model of ocular surface disease. FIG. 2E illustrates representative histology results, showing middle-section by the periodic acid-Schiff (PAS) staining. An analysis of FIG. 2E suggests fewer goblet cells are present in a vehicle treated animal compared to a PLC-treated animal.

FIG. 3 illustrates combined data from the first study and the second study. Each eye was scored independently and compared to its own pretreatment baseline (day 3). Vehicle and PLC arms were pooled between two independent studies, while other arms were performed in the second study only. Error bars=95% confidence interval. Here, PRB v1 PLC refers to PLCs produced using Method 1 described below, PRB v2 MLC lysate refers to MLC lysate produced using Method 2 described below, and PRB v3 MLC lysate refers to MLC lysate produced using Method 3 described below. Statistical analysis was performed for each time point using Kruskal-Wallis ranked sum test, which revealed significant differences between the groups (p<0.0001) at all three time points. Post-hoc testing was performed via Dunn's multiple comparisons test. Multiplicity adjusted P-values were calculated between each group at each time point. Indicated with asterisks are the comparisons where test articles showed significantly improved CFS staining compared to Untreated or Vehicle treated mice. * p<0.05, ** p<0.01, *** p<0.001.

In sum, the analysis of the results indicates that megakaryocyte derivatives (e.g., PLCs, MLC lysate) can improve corneal fluorescein staining (CFS) in subjects with dry eye disease, possibly by ameliorating loss of goblet cells.

### Example 2: Models—TSP1 Knockout Mouse Model of Sjogren's Syndrome

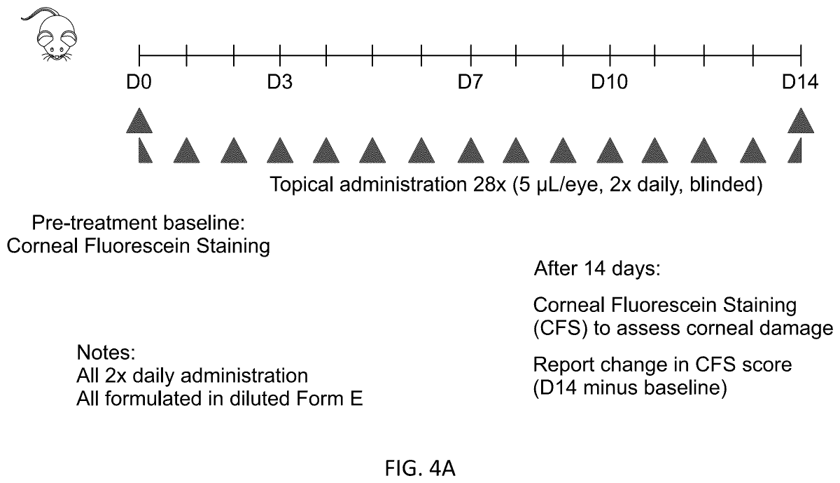

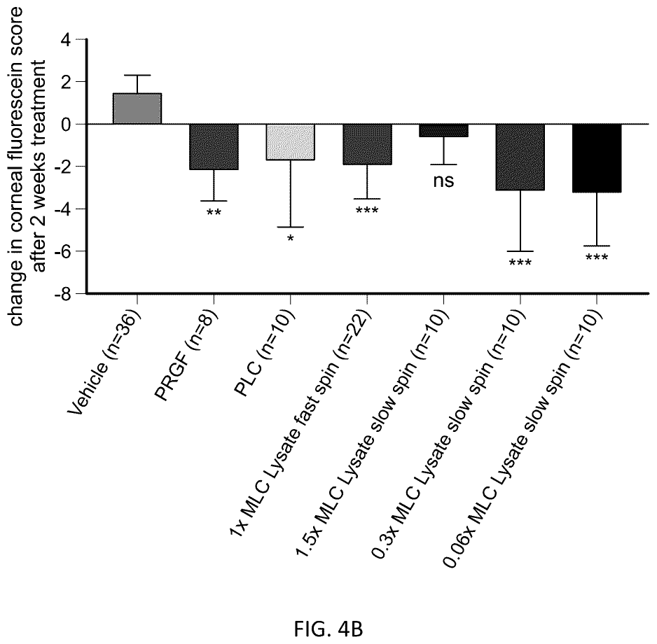

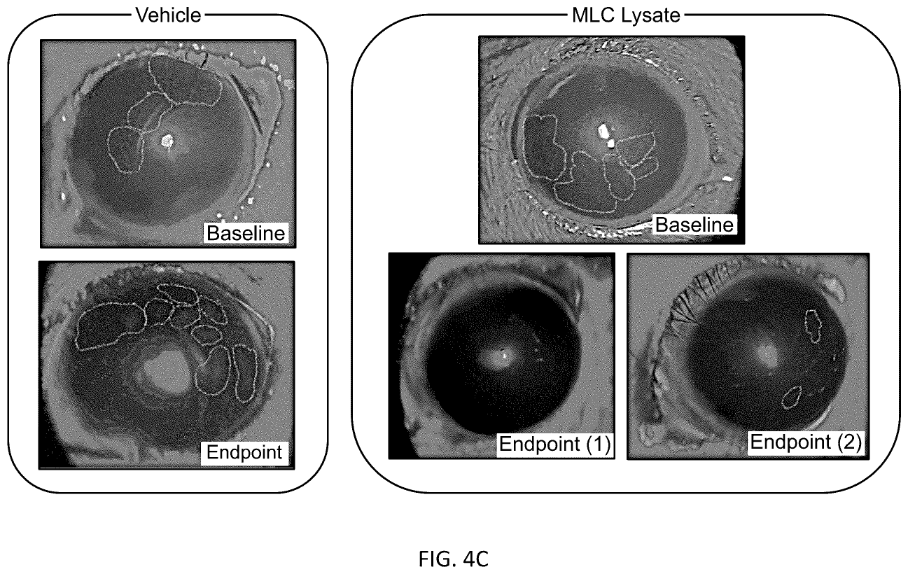

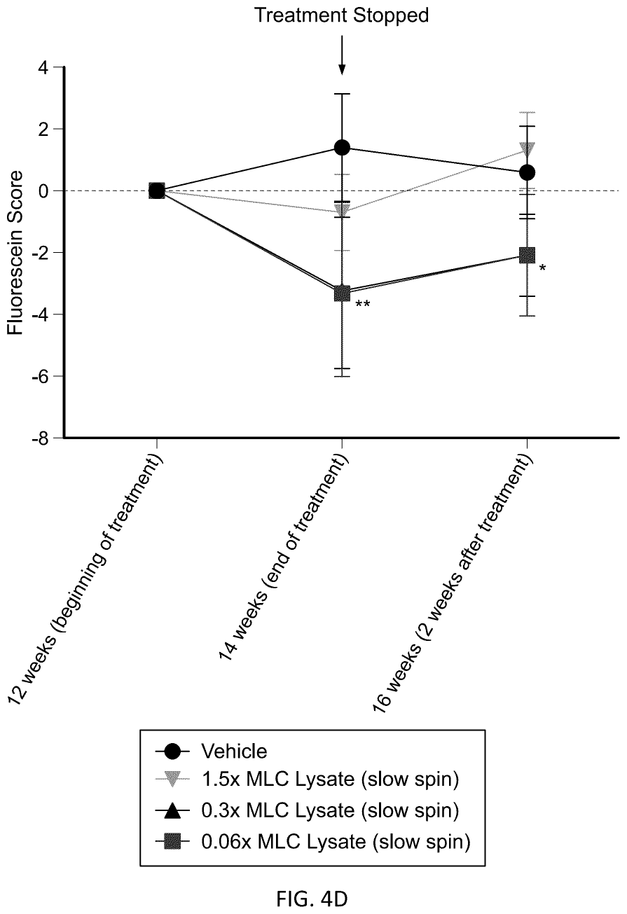

Effects of megakaryocyte derivatives (e.g., PLCs, MLC lysate) in subjects with Sjogren's Syndrome were measured. A TSP1 knockout mouse model of Sjogren's Syndrome was used in an in vivo study as shown in FIG. 4A for testing PLCs. Baseline corneal fluorescein staining (CFS) was performed for each eye on cohorts of 4-6 male TSP1 knockout mice at 12 weeks of age. Mice were treated 2× daily in both eyes for 14 days with PLCs or MLC lysates. PRGF was used as a positive control. Vehicle was used as a negative control. Subsequent to the final treatment, CFS was measured again in each eye and compared to the baseline CFS scores at day 12. Four comparison studies of Study 2 were performed, as outlined as follows. Study 2.1 (n=14 mice, 28 eyes) comprised the following groups: Vehicle (n=8), PLC (n=10) and MLC Lysate (Lot 1 at 21100 g) (n=10). Study 2.2A (n=11 mice, 22 eyes) comprised the following groups: Vehicle (n=10) and MLC Lysate (Lot 2 at 21100 g) (n=12). Study 2.2B (n=9 mice, 18 eyes) comprised the following groups: Vehicle (n=8) and PRGF (n=9). Study 2.3 (n=20 mice, 40 eyes) comprised the following groups: Vehicle (n=10), MLC Lysate (Lot 3 at 200 g) at 1.5× dose (n=10), MLC Lysate (Lot 3 at 200 g) at 0.3× dose (n=10), and MLC Lysate (Lot 3 at 200 g) at 0.06× dose (n=10). Study 2.3 included three doses of MLC Lysates (Lot 3 at 1.5×, 0.3×, and 0.06× the amount of total protein as in the PLC arms of Studies 2.1 and 2.2A) and extended the observation time for 2 additional weeks after the final dosing, to assess duration of effect.

FIG. 4B illustrates pooled results from Study 2. An analysis of FIG. 4B indicates that topical administration of various megakaryocyte derivatives can reverse progression of dry eye disease symptoms in TSP1 knockout mice. The highest dose of MLC lysate (Method 2 at 1.5× the amount of total protein as in the PLC) tested seemed to lose its effect, suggesting that too high concentration may not be ideal and that the optimal dose of MLC lysate is likely lower. ‘MLC Lysate fast spin’ represents pooled data from Method 1. ‘MLC Lysate slow spin’ is from Method 2. One-way ANOVA: p<0.0001 (indicates significant difference among means). Error bars=95% confidence interval. Dunnett's multiple comparisons test (vs Vehicle): *** p<0.001. ** p<0.01. * p<0.05. ns not significant.

FIG. 4C illustrates representative images with marked areas of corneal fluorescein staining from Study 2.2A. An analysis of FIG. 4C indicates that topically applied MLC lysate (twice daily for 2 weeks) significantly improves corneal barrier integrity as compared to vehicle-treated cornea.

FIG. 4D illustrates comparison of fluorescein score from Study 2.3 as a function of time. In Study 2.3, extension of the observation time for 2 additional weeks after the final dosing allowed a preliminary assessment of duration of effect. CFS scoring was performed at 14 weeks of age (i.e. at end of treatment) and again at 16 weeks of age (i.e. 2 weeks after cessation of treatment) and the score of each eye was compared to the baseline score measured at 12 weeks. The results indicate treatment with megakaryocyte derivatives can improve corneal barrier integrity for at least 2 weeks after cessation of treatment. Error bars—95% confidence intervals. *p<0.05, **p<0.01 compared to Vehicle (Mann-Whitney T-test).

Based on analysis of results, it was indicated that megakaryocyte derivatives (e.g., PLCs, MLC lysate) can improve corneal fluorescein staining (CFS) in subjects with Sjogren's Syndrome. It was further indicated that positive effects of the treatment may continue to exist even after the treatment is stopped. Accordingly, Sjogren's Syndrome can be treated with megakaryocyte derivatives (e.g., PLCs, MLC lysate) using a drug holiday treatment protocol.

### Example 3: Methods of Making Megakaryocyte Derivatives

Described herein are various methods for making megakaryocyte derivatives.

Method 1: The method was used to produce PLCs. Briefly, the method was performed in a microfluidic bioreactor (BioR). About 3.2e9 MLCs were thawed and centrifuged to remove DMSO. The MLCs were resuspended to a concentration of ˜3.33e7/ml with CO₂-independent media with 2% Pluronics F127. MLCs were then subjected to spin priming process by incubating 480 mL of resuspended MLCs into a PBS 0.5 vessel at 35 rpm for 18 hours. Spin-primed MLCs were seeded into 2×16-layer 3 μm pore platelet bioreactors. System was flushed with 375 mL of media to ensure complete collection of MLC derived products. Tangential flow filtration (TFF) was performed to concentrate and diafiltrate products. The product was diafiltrated 7 times into Plasmalyte, followed by addition of Trehalose to 250 mM for cryostorage. The product was aliquoted into single-use vials for each treatment and stored at −80° C. Each treatment vial was thawed at room temperature (RT) immediately before use.

Method 2: Also referred herein as MLC Lysate Lots 1 and 2, the method was used to produce MLC Lysate processed by a high speed (21100 g) centrifugation. Briefly, MLCs were thawed and centrifuged to remove DMSO. The MLCs were then resuspended with 150 mM Trehalose in a mixture of Plasmalyte and water at either 5e7/mL (for standard dose, both Lots 1 and 2) or 2e8/mL (for max dose, for Lot 1 only). Freeze-thaw lysis was performed by cycling incubation of the MLCs at −80° C. and RT 3 times. The MLC Lysate was vortexed between each freeze-thaw cycle. The MLC Lysate was clarified by centrifuging at high speed (21,100×g). MLC Lysate was collected as supernatant. BCA total protein analysis was run to determine the total protein concentration of the MLC Lysate and results were used to determine in vivo dosing. The MLC Lysate were aliquoted into single-use vials for each treatment and stored at −80° C. Each treatment vial was thawed at RT immediately before use.

Method 3: Also referred herein as MLC Lysate Lot 3, the method was used to produce MLC Lysate processed by a low speed (200 g) centrifugation. Briefly, MLCs were thawed and centrifuged to remove DMSO. The MLCs were then resuspended with 150 mM Trehalose in a mixture of Plasmalyte and water at either 5e7/mL (for standard dose) or 2e8/mL (for max dose). Freeze-thaw lysis was performed by cycling incubation of the MLCs at −80° C. and RT 3 times. The MLC Lysate was vortexed between each freeze-thaw cycle. The MLC Lysate was then clarified by centrifuging at slow speed (200×g). The clarified lysate was left at full strength or a portion further diluted 5× or 25× with 150 mM Trehalose in a mixture of Plasmalyte and water. The different non-diluted and diluted MLCs Lysates was aliquoted into single-use vials for each treatment and stored at −80° C. Each treatment vial was thawed at RT immediately before use.

Method 4: The method was used to produce MLC Lysate processed through a 0.65 um filter. Briefly, MLCs were thawed and centrifuged to remove DMSO. The MLCs were then resuspended with 150 mM Trehalose in a mixture of Plasmalyte and water at 5e7/mL. Freeze-thaw lysis was performed by cycling incubation of the MLCs at −80° C. and RT 3 times. The resulting product solution was clarified by being vacuum filtered through a 20 μm Überstrainer. The resulting product solution was further clarified by being pushed through a 0.65 μm polycarbonate track etch (PCTE) membrane in a filter cassette. Filtered product was then aliquoted and stored at −80° C.

Method 5: The method was used to produce MLC Lysate processed through a 5 μm filter. Briefly, MLCs were thawed and centrifuged to remove DMSO. The MLCs were then resuspended with water at 5e7/mL. Freeze-thaw lysis was performed by cycling incubation of the MLCs at −80° C. and RT 3 times. The resulting product solution was clarified by being pushed through a syringe filter cassette containing a 20 μm PCTE membrane. The resulting product solution was further purified by being pushed through a 5 μm PCTE membrane in a second filter cassette. Final product was formulated to contain 150 mM Trehalose and then aliquoted and stored at −80° C.

Method 6: The method was used to produce MLC Lysate processed through a 0.22 um filter. Briefly, MLCs were thawed and centrifuged to remove DMSO. The MLCs were then resuspended with water at 5e7/mL. Freeze-thaw lysis was performed by cycling incubation of the MLCs at −80° C. and RT 3 times. The resulting MLC Lysate was clarified by being pushed through a syringe filter cassette containing a 20 μm PCTE membrane. The MLC Lysate was further clarified by being pushed through a 5 μm PCTE membrane in a second filter cassette. As a final step, the resulting product solution was passed through a 0.22 μm PES filter. Final product was formulated to contain 150 mM Trehalose and then aliquoted and stored at −80° C.

### Example 4: Methods of Making Platelet Rich in Growth Factors (PRGF)

Described herein is a method of making platelet rich in growth factors (PRGF). Briefly, donor blood was collected and used for isolation of platelet rich plasma (PRP) and the further processed to make PRGF, following approved guidance and protocols. Donated whole blood was first centrifuged at 150×g to enable the harvest of plasma from red blood cells and buffy coat. Prostaglandin E1 (PGE1) was added to the plasma sample and was centrifuged at 460× g to concentrate platelets. Platelet-poor fraction (upper ⅔ portion) was removed and discarded after centrifugation. Pellet was resuspended with the remaining lower ⅓ portion of plasma. The resulting PRP was then activated by adding 228 mM CaCl₂) and incubating at 37° C. for at least 15 minutes or until clot formation was observed. Activated PRP was centrifuged at 16600×g. Supernatant was isolated and diluted to 20% (v/v) with saline. Final product was aliquoted into single-use vials and stored at −80° C. Single-use vials was thawed at RT immediately before use.

### Example 5: Profiling Growth Factors, Cytokines, and Other Relevant Proteins in MLCs and MLC Derivatives

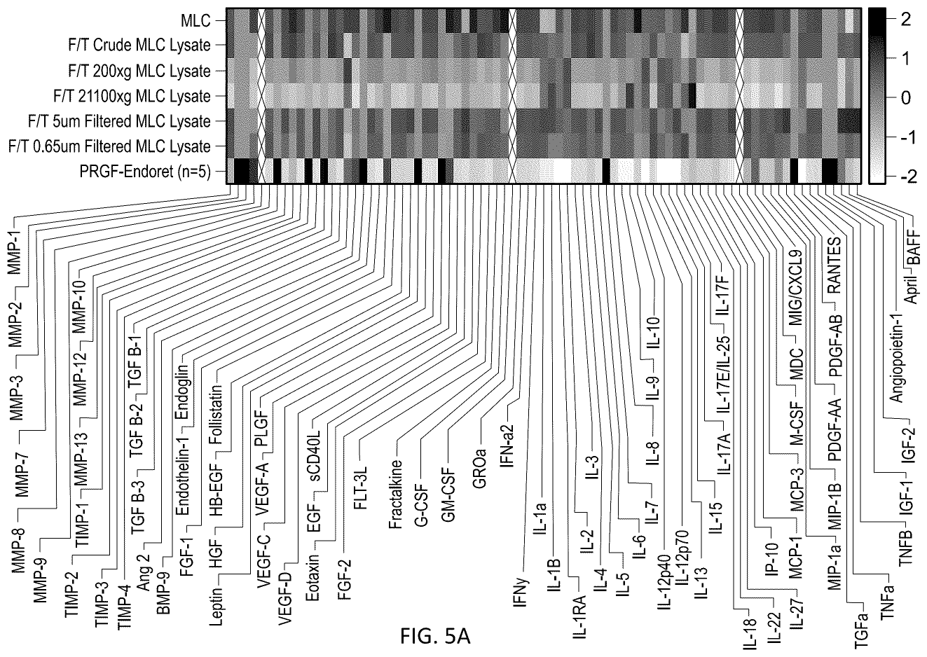

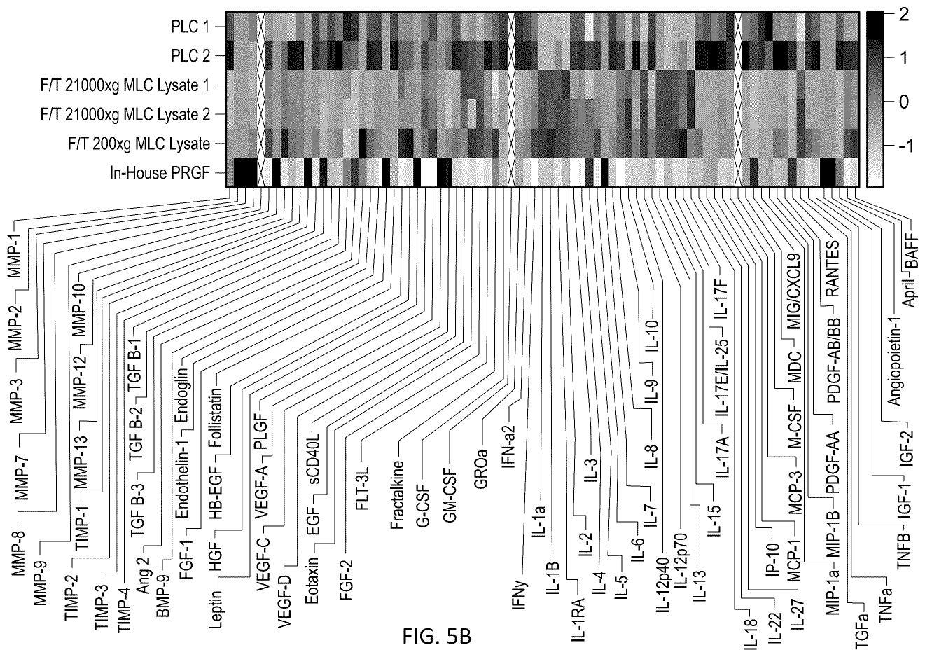

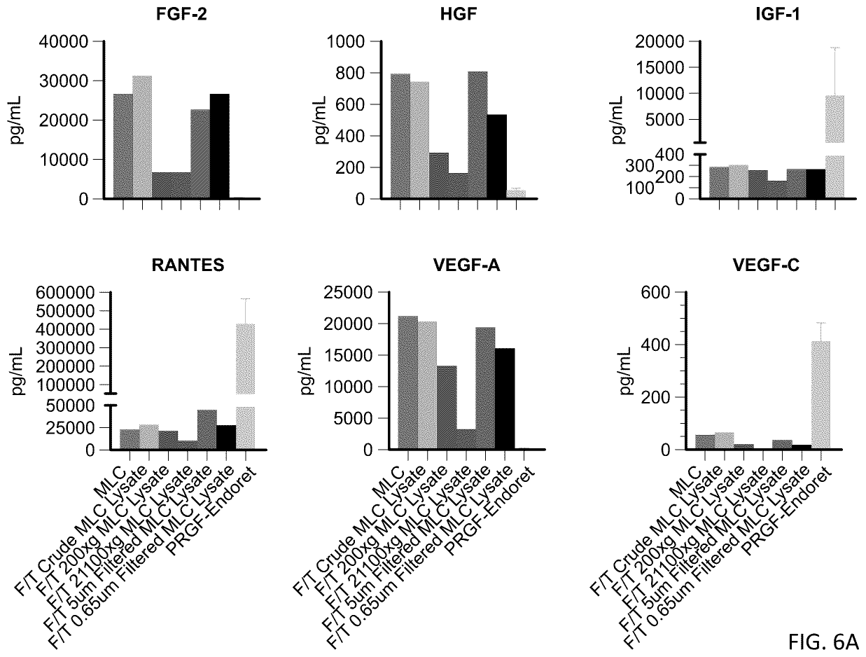

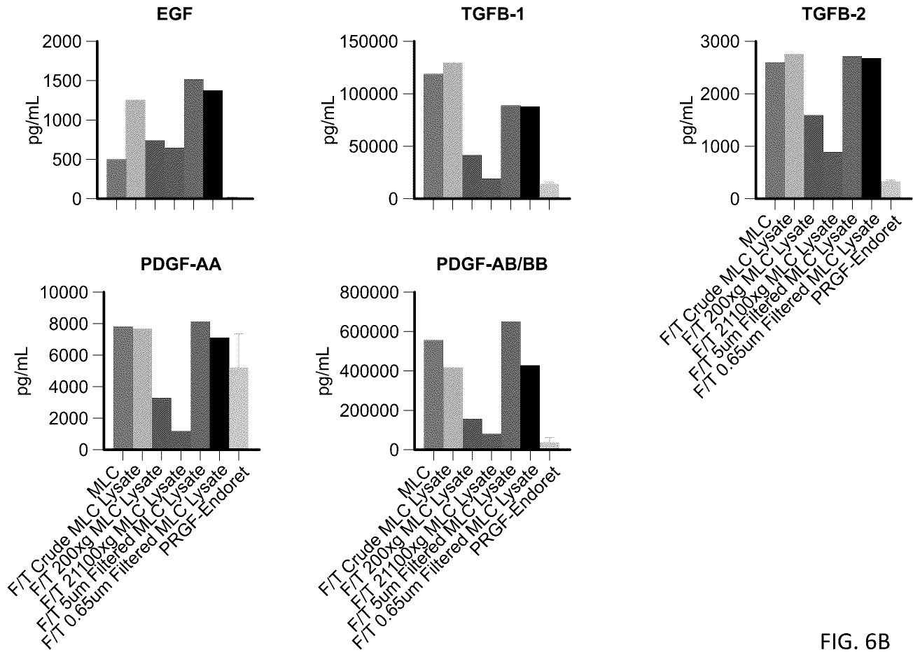

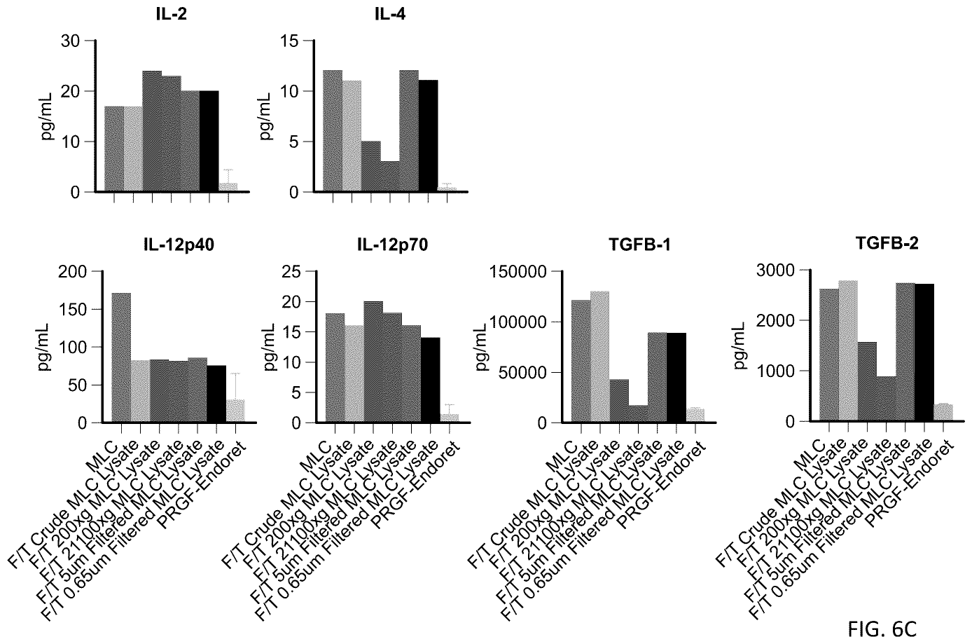

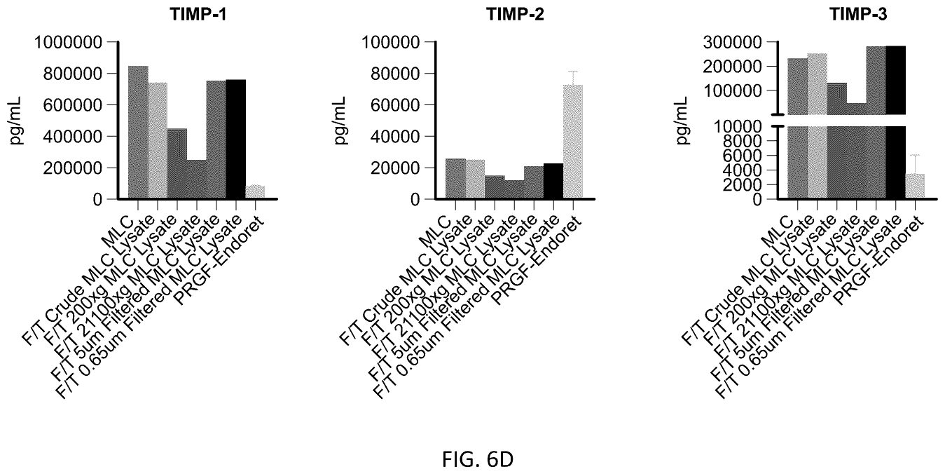

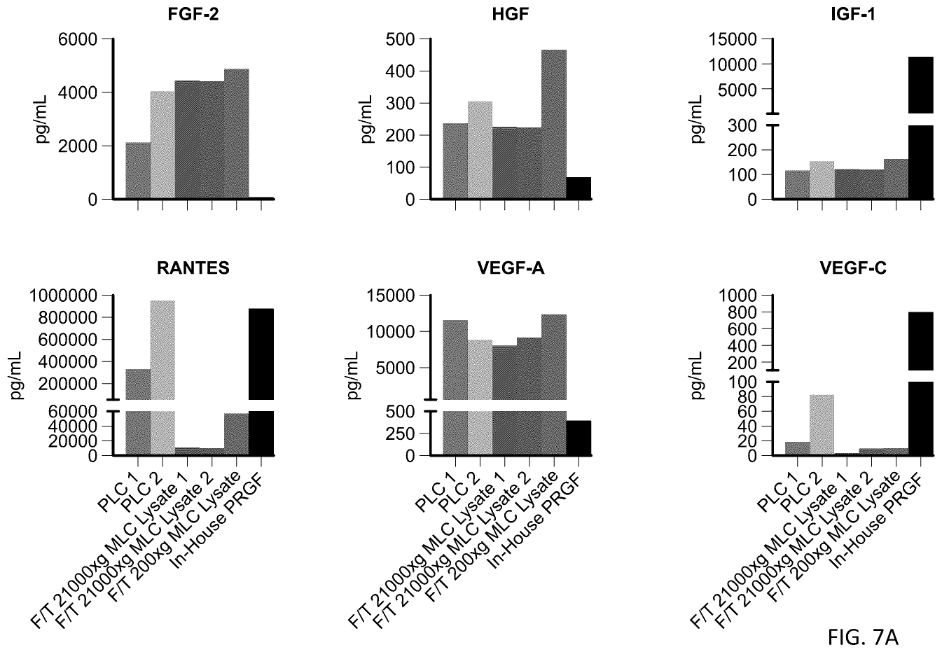

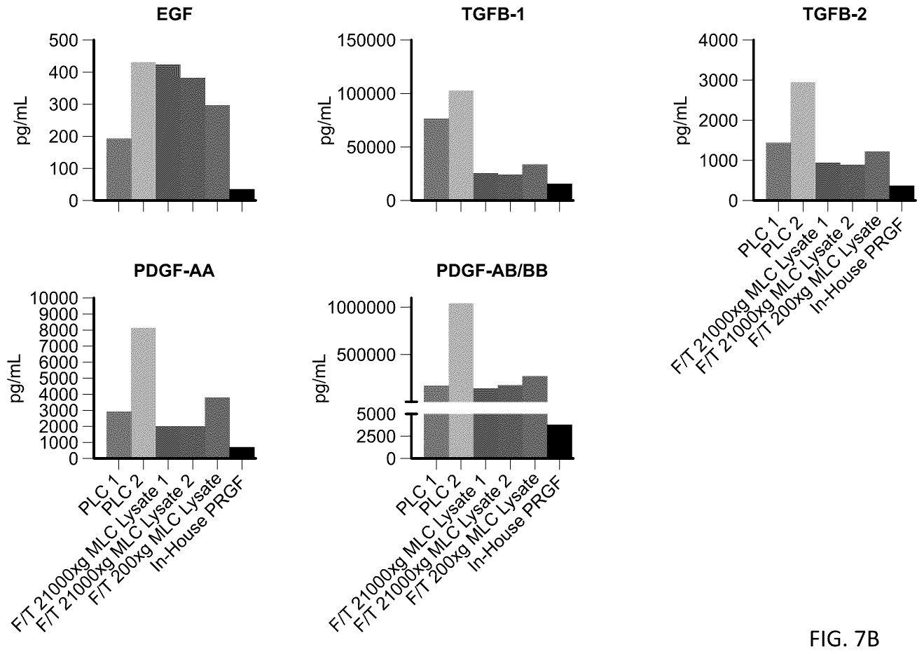

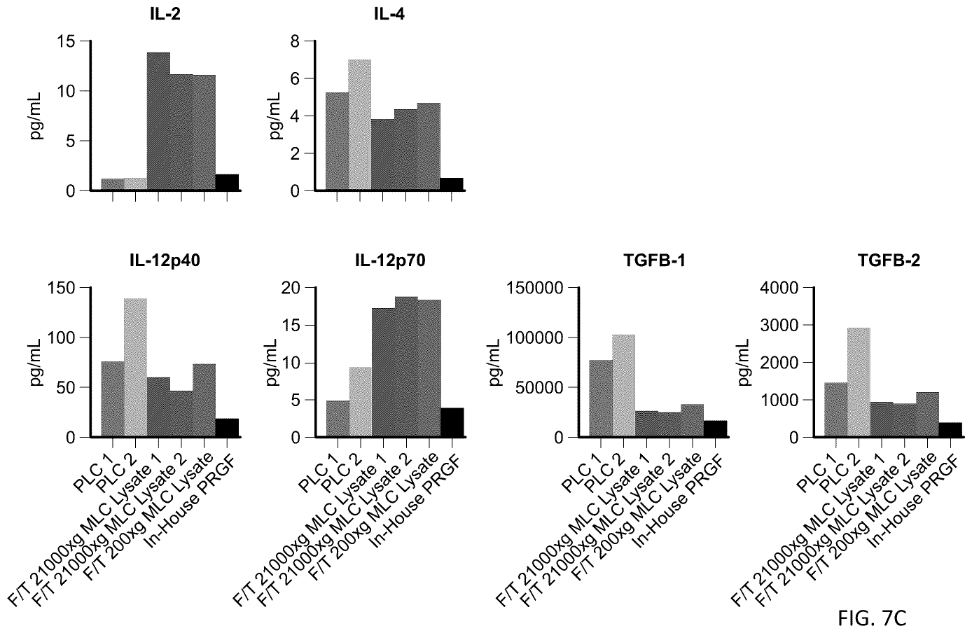

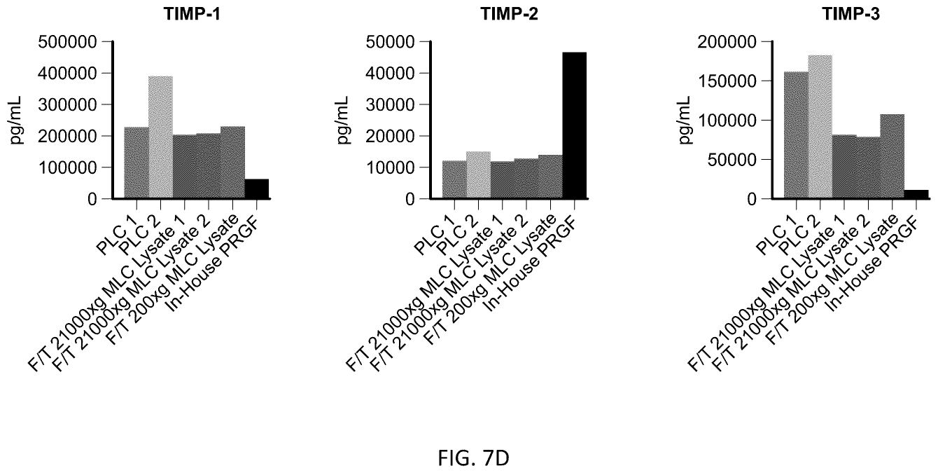

Profiling for growth factors, cytokines, and other relevant proteins was determined for MLCs, Crude MLC Lysate (i.e. without further processing), PLC, and MLC Lysates that were processed in different ways. PLC was generated using Method 1 as described in Example 3. MLCs were lysed and processed using Method 2, Method 3, Method 4 and Method 5, described in Example 3. ‘In-house’ PRGF (plasma rich in growth factors) was prepared using the method described in Example 4, while ‘Endoret-PRGF’ was obtained from the manufacturer who prepared it from human blood using the Endoret Eye Drop Kit. In preparation for biomarker profiling, each sample was incubated with RIPA Lysis Buffer (10×RIPA, 100×PI), followed by 3 cycles of 1 minute max-force vortex and 10 minutes incubation on ice, followed by clarification via high-speed centrifugation. Total Protein concentration of all the clarified lysates were measured using the Pierce BCA Assay. The samples were then diluted to a final concentration of 1 mg/mL total protein with Plasmalyte-A for analysis. Additional dilutions of up to 500-fold were applied to samples to accommodate range of human panel assays. Growth factor and cytokine profiling was used to simultaneously analyze multiple biomarkers (70+ cytokines, GFs, and metalloproteases) at once. PRGF (plasma rich in growth factors, a PRP releasate) was used as a control. FIG. 5 is a heat map representation of profiled cytokines and growth factors, illustrating the relative concentration of the cytokines and growth factors as measured by the profiling. The data presented represents the Z-scores of full-strength concentrations of growth factors and cytokines per volume of sample. FIGS. 6A-6D and 7A-7D show the actual concentrations of selected factors, and indicates that various types of MLC contain high concentrations of desirable factors (Epitheliotrophic and Platelet Associated Factors, TIMPS that inhibit MMP tissue degradation, Interleukins that polarize T-cells to secrete pro-resolutory cytokines) compared to PRGF from human blood. Accordingly, MLCs and lysates thereof are distinct products relative to PRGF.

In sum, the analysis of the results indicates that MLCs and derivatives thereof can offer unique therapeutic advantage relative to PRGF.

### Example 6: Biomarker Profiling of Megakaryocyte Derivatives Using Enzyme-Linked Immunosorbent Assay (ELISA)

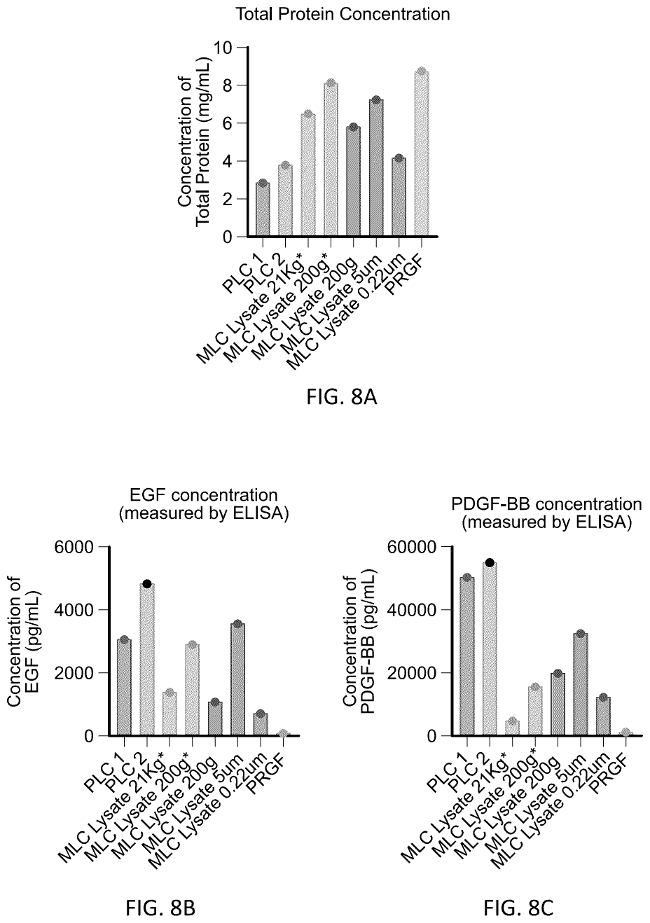

Biomarker profiling was determined for megakaryocyte derivatives (e.g., MLC lysates and PLCs) using enzyme-linked immunosorbent assay (ELISA) for samples prepared in examples 5 and 6. Briefly, total protein concentration for each sample was determined using a total protein analysis (BCA). FIG. 8A illustrates results of the BCA for all the samples that were tested. The results indicate that megakaryocyte derivatives contain different overall protein levels depending on how they were produced/processed. * Denotes a representative pair of samples that were produced with the same process as the methods for the in vivo study.

FIGS. 8B and 8C provide ELISA-based quantification of EGF and PDGF-BB, respectively, in various megakaryocyte derivatives. An analysis of FIG. 8B indicates that EGF profiling of samples show similar trends as observed in examples 5 and 6. * Denotes representative samples that were produced and processed with the same process as Method 2 (21100 g) and Method 3 (200 g). Grey bar is Method 3 (200 g).

Based on analysis of ELISA results, it was further confirmed that MLC lysates and PLCs comprise unique biomarker profile relative to PRGF. Accordingly, MLC lysates and PLCs can offer unique therapeutic advantage relative to PRGF.

### Example 7: Particle Size Analysis of Megakaryocyte Derivatives

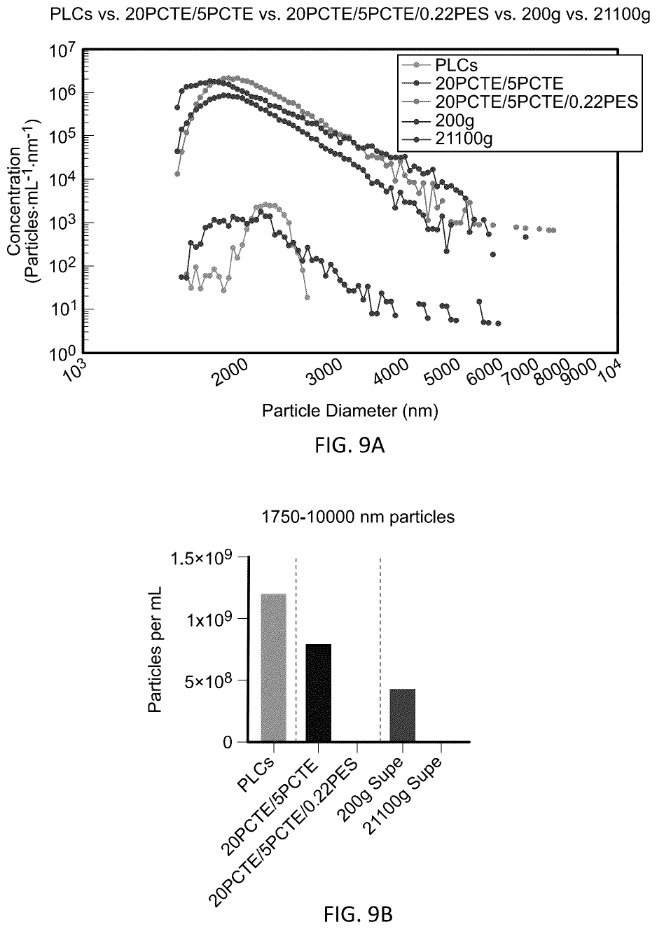

Particle size analysis was performed for different megakaryocyte derivatives (e.g., MLC lysates and PLCs). Briefly, particle size distributions was measured by an nCS1 particle analyzer (Spectradyne, Signal Hill, CA) of PLCs and crude MLC lysate filtered through a 20 μm PCTE/5 μm PCTE membrane or a 20 μm PCTE/5 μm PCTE/0.22 μm PES membrane, and the supernatant of MLC lysate centrifuged at 200 g or 21,100 g. Samples were diluted 5- to 200-fold in a 0.22 μm-filtered diluent (1×PBS with 1% Tween-20). Diluted samples were then loaded into a C-10k nCS1 analysis cartridge. Data were acquired for N>6000 unless indicated otherwise. Raw data were then processed in Viewer™ software (Spectradyne). FIG. 9A illustrates results of the particle size distribution of 5 samples that were tested. FIG. 9B illustrates concentrations of particles (1750-10000 run) in each sample represented as bar graphs. Virtually all micro-sized particles are absent from 21100 g centrifuged and 0.22 μm filtered megakaryocyte derivatives. This shows that depending on processing steps, megakaryocyte derivatives may be generated that are predominantly particulate in nature (PLC), predominantly soluble in nature (21100 g supernatant and 0.2 μm filtered), or a combination of both (200 g supernatant and 5 μm filtered). Thus, the megakaryocyte derivatives prep may be tailorable to match the properties desired.

### Example 8: Effects of Megakaryocyte Derivatives on Human Corneal Epithelial (HCE-T) Cell Proliferation

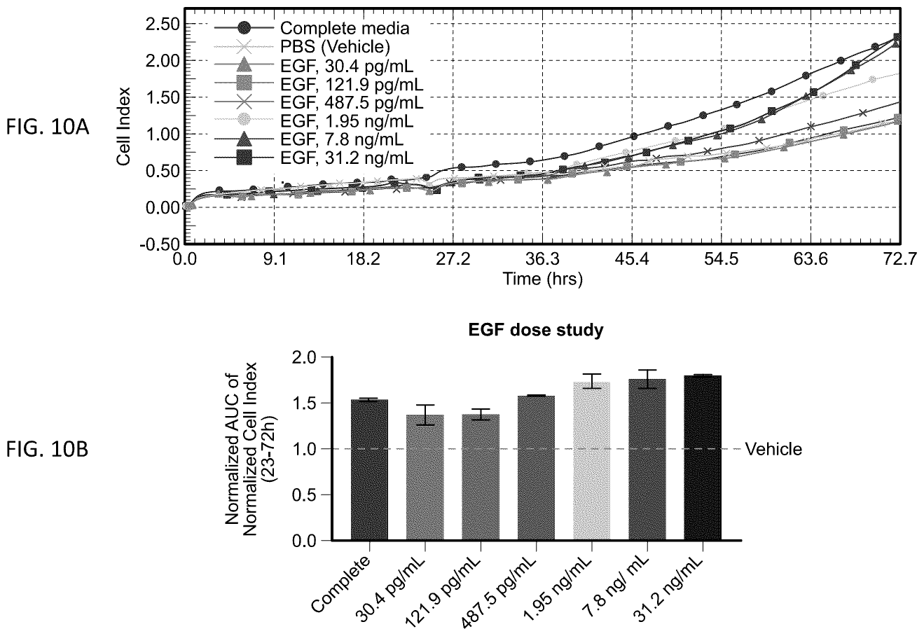

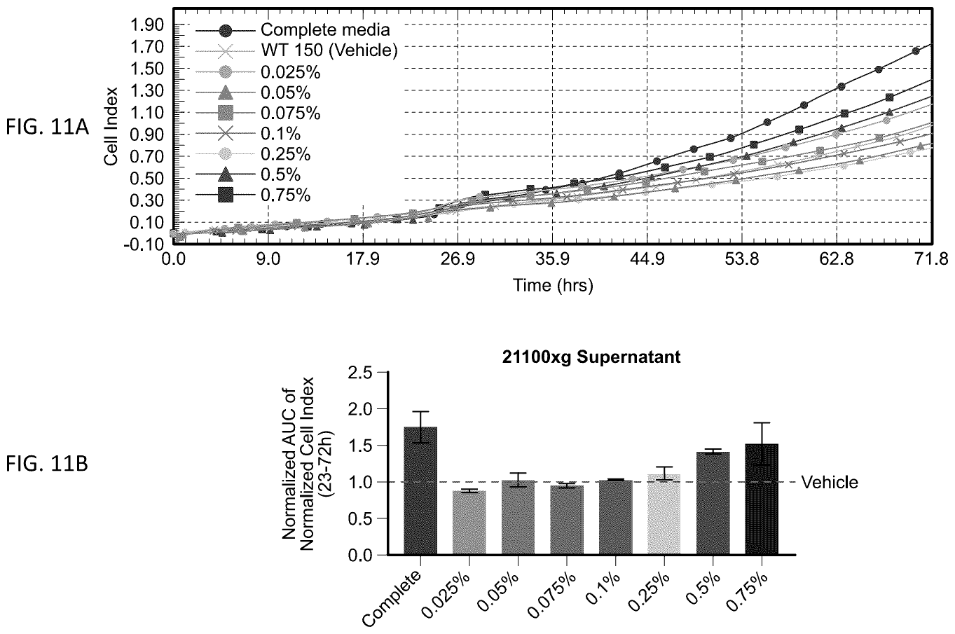

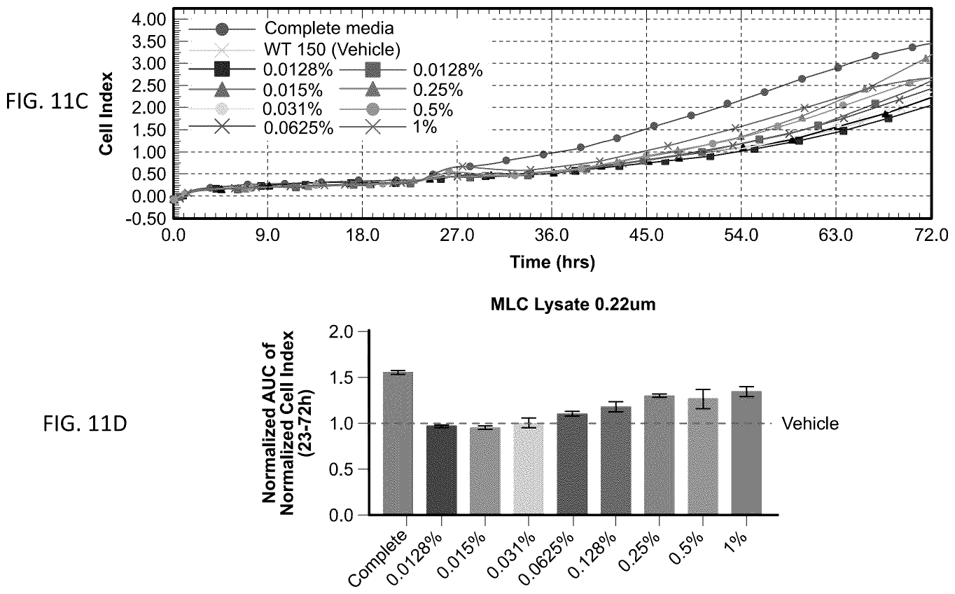

Effect of MLC lysate on proliferation of human corneal epithelial (HCE-T) cells was measured. Briefly, HCE-T cells were plated on to E-Plate 96 (Agilent Technologies, Santa Clara, CA) at 2,500 cells per well in minimal KGM-Gold medium (Lonza, Walkersville, MD). Minimal KGM-Gold medium contains all KGM-Gold BulletKit components except for EGF (positive control), MLC lysates, and/or bovine pituitary extract. For negative control vehicle only, phosphate buffer solution (PBS) was used. After 24 hours, cells were treated with indicated concentrations of MLC lysates, EGF (SinoBiological Inc., Beijing, China) or PBS. The E-plate was then returned to the xCELLigence RTCA instrument for data collection for an additional 48 hours. Area under the curve (AUC) of normalized Cell Index was calculated using RTCA Software Pro and then normalized to vehicle. FIG. 10A illustrates results of a 72-hour time course study of effects of EGF on proliferation of HCE-T cells at a concentration ranging from 30.4 pg/ml to 31.2 ng/ml. FIG. 10B is an alternate representation of FIG. 10A showing normalized area under curved of normalized cell index for each concentration. An analysis of FIGS. 10A-10B indicate that EGF promotes corneal epithelial cell proliferation in a dose-dependent manner. FIG. 11A illustrates results of a 72-hour time course study of effects of MLC Lysates processed with high speed centrifugation on the proliferation of HCE-T cells at a dose ranging from 0.025% (v/v) to 0.75% (v/v). FIG. 11B is an alternate representation of FIG. 11A showing normalized area under the curve of normalized cell index for each concentration. FIG. 11C illustrates results of a 72-hour time course study of effects of MLC Lysates processed via 5 μm filtration followed by 0.22 μm filtration on the proliferation of HCE-T cells at a dose ranging from 0.0128% (v/v) to 0.1% (v/v). FIG. 11D is an alternate representation of FIG. 11C showing normalized area under the curve of normalized cell index for each concentration. An analysis of FIGS. 11A-11D indicates that the proliferation of HCE-T cells can be enhanced by addition of MLC lysates, validating their biological activity and demonstrating a potential mechanism of action contributing to alleviation of ocular surface disease.

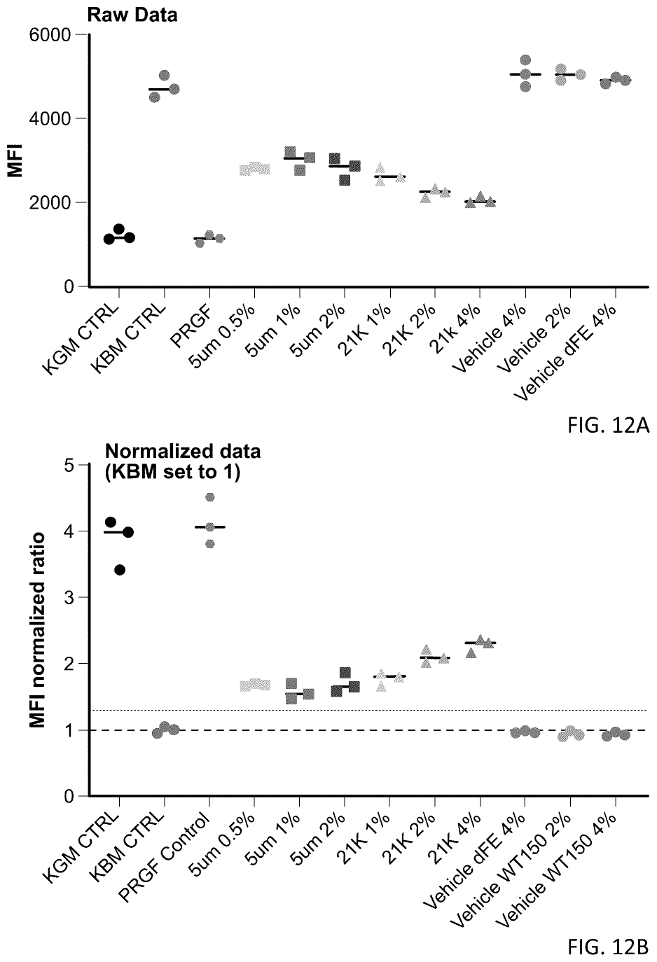

To further confirm proliferative effects of megakaryocyte derivatives on HCE-T cells, an orthogonal assay, carboxyfluorescein diacetate succinimidyl ester (CFSE)-based flow cytometry assay, was performed. Briefly, HCE-T cells were labeled with CFSE (Invitrogen Cell Trace™), plated at 10,000 cells per well and grown in complete media (KGM-Gold, Lonza). Media was changed to basal media (KBM-Gold, Lonza), MLC lysates produced through use of 5 μm filter (5 μm 0.5%-2%), 21,100× g centrifugation (21 k 2%-4%), or corresponding vehicles were added to cells and incubated for an additional 60-72 hours. Assay controls include complete media, basal media and PRGF, and were all incubated for the same period of time as test articles. Miltenyi MACSquant flow cytometer was used for recording geometric mean fluorescence intensity (MFI) for CFSE labelled cells. FIG. 12A shows MFI observed for each tested condition, proliferation is inversely related to MFI value. FIG. 12B is an alternate representation of FIG. 12A showing MFI normalized to the KBM control and inversed, now showing direct correlation between increased proliferation and normalized MFI value. Samples with values of 1-1.3 (dotted line) show no increase in proliferation. Samples above 1.3 (dotted line) indicate increased proliferation when compared to negative control for each tested condition. An analysis of FIGS. 12A-12B indicates that the proliferation of HCE-T cells can be enhanced by treatment with megakaryocyte derivatives, validating its biological activity and demonstrating a potential mechanism of action contributing to alleviation of ocular surface disease symptoms.

In sum, the analysis of the results indicates that unique biomarker composition of MLC lysates advantageously helps alleviated ocular surface disease symptoms by promoting proliferation of HCE-T cells.

### Example 9: Osteoarthritis Model for In Vivo Efficacy Study

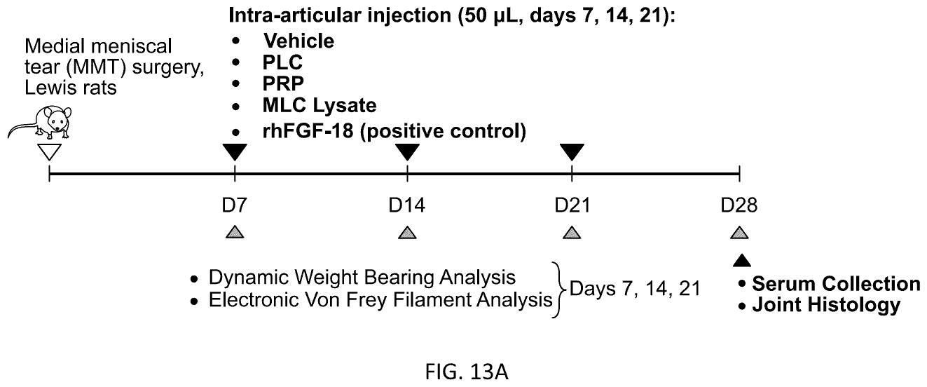

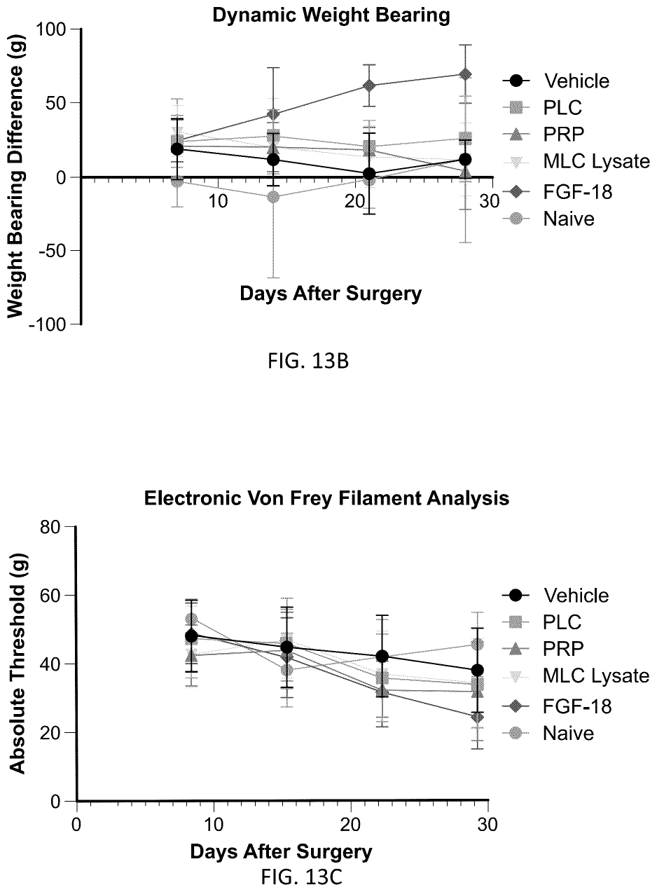

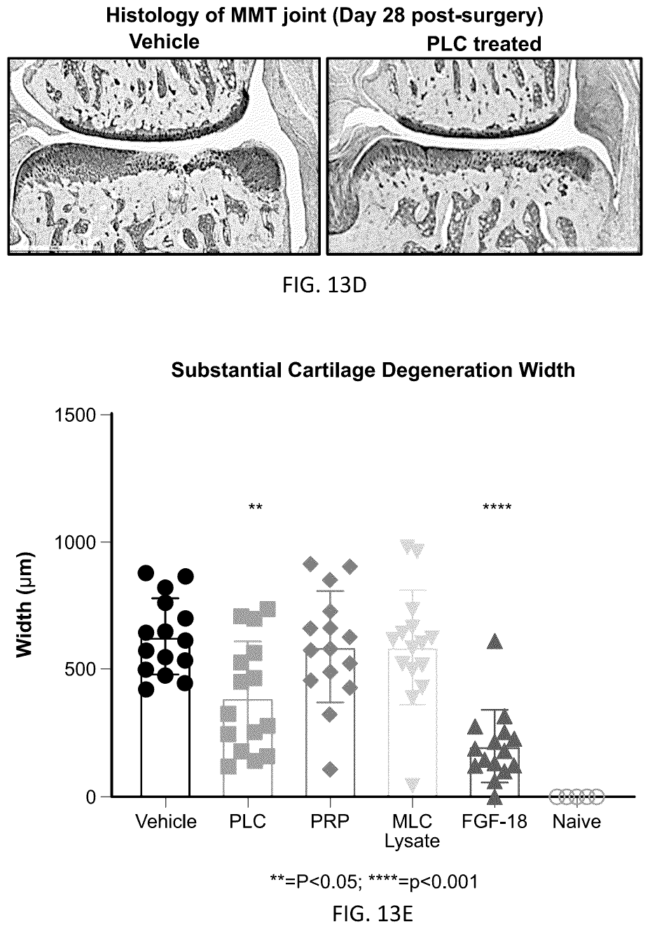

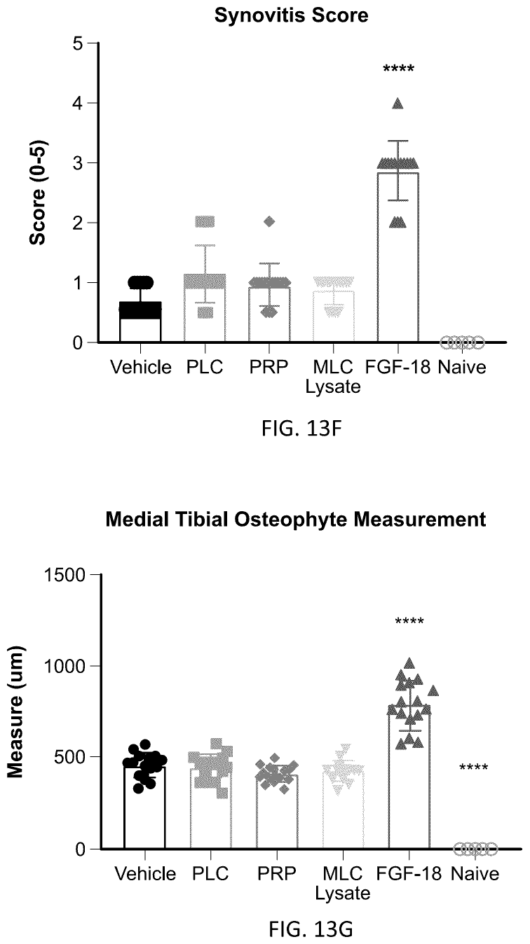

In vivo effect of MLC lysate and PLC in a subject having osteoarthritis was measured. A rat medial meniscal tear (MMT) model of osteoarthritis (OA) was used in an in vivo study for testing efficacy of megakaryocyte derivatives. FIG. 13A shows an outline of the study design. As described in FIG. 13A, Lewis rats were subjected to meniscal tear surgery on day 0. Dynamic weight bearing and electronic Von Frey Filament analyses were conducted at 7, 14, 21 and 28 days following surgery. Subjects were administered with MLC lysates or PLCs by intraarticular injection after DWB and EVFF analysis on days 7, 14 and 21 following surgery. On day 28 following surgery, serum was collected, and animals were sacrificed to allow for joint histology. PRP and recombinant human FGF-18 were used as positive controls. For negative control vehicle only was used. In sum, the study comprised the following groups: No surgery (n=5), MMT Surgery+Vehicle (n=15), MMT Surgery+Platelet-like cells (n=15), MMT Surgery+Platelet-rich plasma (n=15), MMT Surgery+rFGF-18 (n=15), and MMT Surgery+MLC Lysate (n=15).

FIGS. 13B and 13C illustrate efficacy results of MLC lysate and PLC in the rat medial meniscal tear model of osteoarthritis using dynamic weight bearing test and electronic Von Frey Filament test, respectively. As shown in FIG. 13B, MLC lysate, PLCs, and PRP show an improved pain profile as compared to recombinant human FGF-18, which shows a progressive increasing signal over time in dynamic weight bearing testing as compared to vehicle. In electronic von Frey filament analysis, as shown in FIG. 13C, PLC and MLC lysate treatment resulted in thresholds comparable to vehicle treatment while recombinant human FGF-18 showed a significant decrease in thresholds indicating higher pain sensitivity.

As outlined in FIG. 13A, a histological analysis of test subjects was performed 28 days following the surgery. FIG. 13D shows representative H&E-stained histological images of the vehicle and PLC-treated treated joints. A disruption in articular cartilage was observed in the vehicle treated joint (left) while the articular cartilage layer was intact on the PLC treated joint (right). FIG. 13E shows quantitative representation of cartilage degeneration observed in the histological analysis of in each subject. As illustrated in FIG. 13E, a statistically significant reduction in substantial cartilage degeneration width is observed with PLC treated (p=<0.05) compared to vehicle. Treatment with recombinant human FGF-18 also shows a significant reduction compared to vehicle treatment (p<0.001). Additionally, synovitis score wad medial tibial osteophyte measurement was performed based on analysis of histological data. FIG. 13F shows assessments of synovitis within the joints of treated subjects. An analysis of FIG. 13F indicates that PLC, PRP and MLC lysate did not show an appreciable difference compared to vehicle treatment, while recombinant human FGF-18 showed a significant increase in synovitis score (****=p, 0.0001). FIG. 13G shows result of quantification of osteophytes within the treated joints of subjects. An analysis of FIG. 13G indicates that PLC, PRP and MLC lysate did not show an appreciable difference from vehicle treatment in medial tibial osteophyte measurement, while recombinant human FGF-18 treatment resulted in a significant increase (****=p<0.0001). Naïve mice were observed to have a significant decrease compared to vehicle treated mice (****=p<0.0001) in medial tibial osteophyte measurement.

In sum, the analysis of the results indicates that unique biomarker composition of PLCs advantageously helps in preserving intact articular cartilage and/or improving/reversing cartilage degeneration condition in osteoarthritis subjects.

### Example 10: Effects of PLCs on Human Umbilical Vein Endothelial Cells (HUVEC) Migration

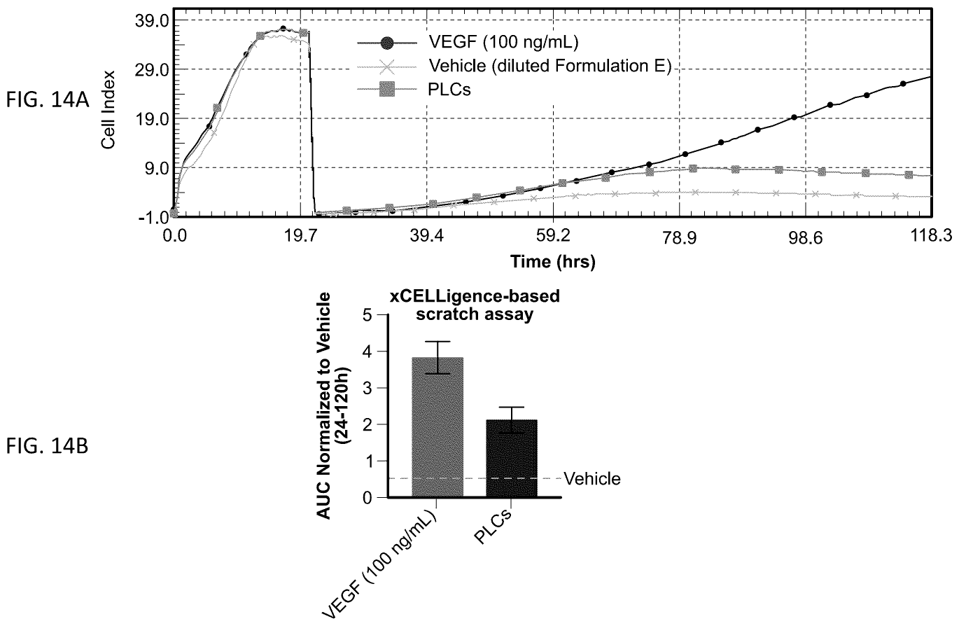

In vivo effect of PLCs on human umbilical vein endothelial cells (HUVEC) migration was measured. Briefly, a rat medial meniscal Scratch assay was performed to evaluate effects of PLCs on HUVEC migration. HUVECs (10,000 cells per well) were plated on to E-Plate Wound 96 (Agilent Technologies, Santa Clara, CA) in Complete EGM-2 medium (Lonza, Walkersville, MD) and incubated for 24 hours in an xCELLigence Real-Time Cell Analysis instrument (Agilent Technologies). A scratch was then generated across each well using an AccuWound 96 Scratch Tool (Agilent Technologies). Wells were rinsed with PBS and replaced with minimal media (EGM-2 without VEGF, EGF, or IGF and containing 0.1% FBS and 1 ng/mL bFGF). An E-plate insert (Agilent Technologies) was placed on the wells and the following treatments were applied on the upper chamber of the insert: vehicle, recombinant VEGF (100 ng/mL), or PLCs*. Recombinant VEGF used as a positive control. PLC samples were produced with a similar process as described in Method 1 of Example 3. The E-plate was then returned to the xCELLigence RTCA instrument for data collection for an additional 96 hours. FIG. 14A illustrates the time course data (120 hours) of the resulting cell index, and FIG. 14B illustrates AUC data normalized to the vehicle. This data shows that PLCs can promote HUVEC migration in a scratch assay, validating the biological activity of megakaryocyte derivatives and demonstrating a potential mechanism of action contributing to wound healing.

In sum, the analysis of the results indicates that unique biomarker composition of PLCs advantageously helps in wound healing of subjects by promoting HUVEC migration.

### Example 11: In Vivo Efficacy of Megakaryocyte Derivatives in Wound Healing

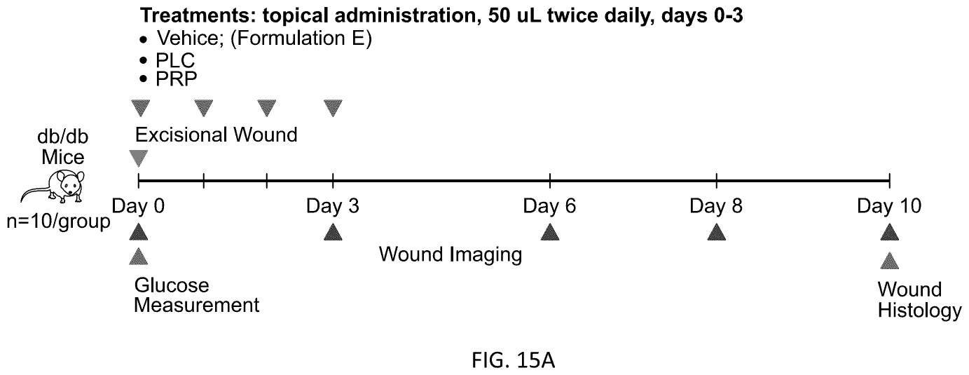

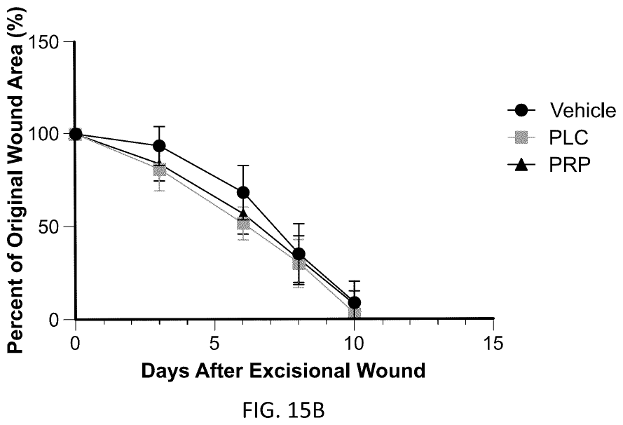

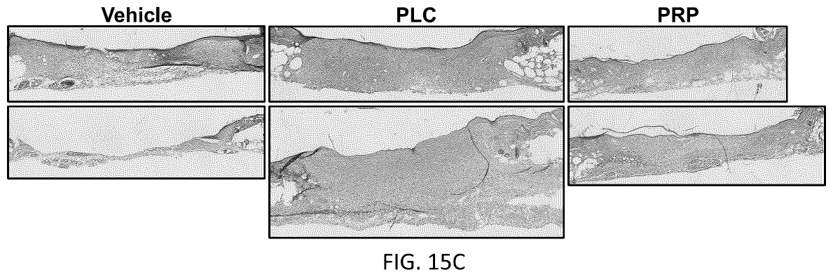

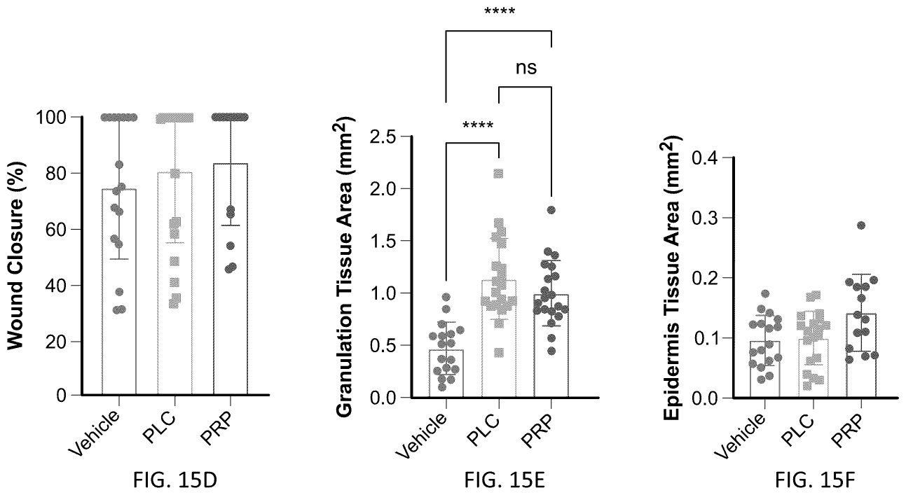

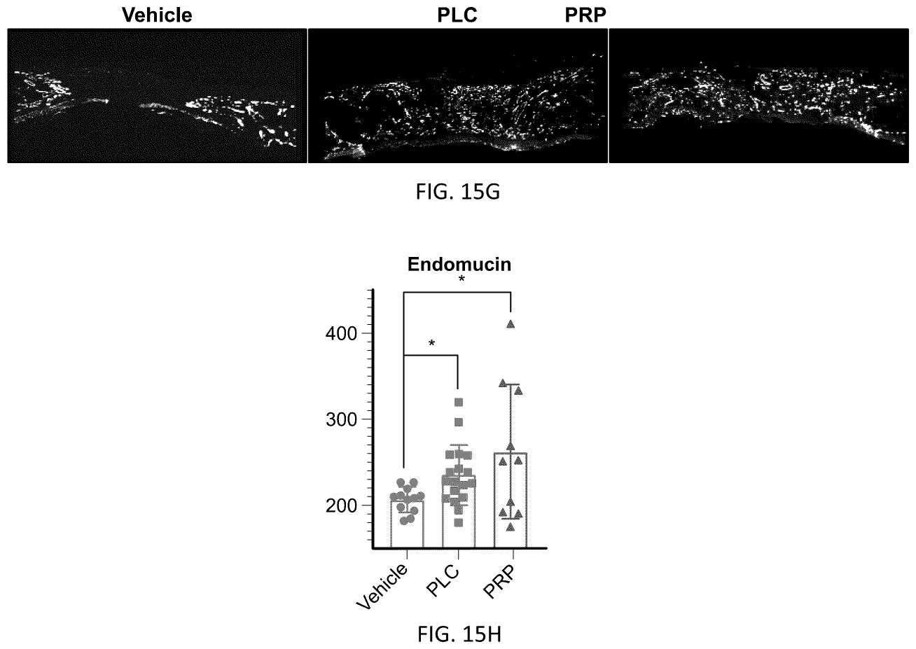

In vivo effect of PLCs on wound healing in diabetic subjects was measured. Briefly, db/db diabetic mouse model was used in an in vivo study for evaluating efficacy of megakaryocyte derivatives (PLCs and MLC Lysates) in wound healing. FIG. 15A shows an outline of the study design. As described in FIG. 15A, on day 0, glucose measurements were performed to ensure mice were in a diabetic state. Mice then underwent surgery to generate two identical excisional wounds via biopsy punch on their backs. Immediately following surgery and for three subsequent days, 50 μL of PLCs was topically administered twice daily for three days. PRP was used as a positive control. For negative control, vehicle only was used. Digital imaging was used to capture wound images on the day of surgery and days 3, 6, 8 and 10 following surgery. On day 10 following in life measurements, animals were sacrificed, and wounds were excised for histological analyses.

Digital imaging and subsequent measurement of wound area was performed over the course of 10 days following test sample treatment. FIG. 15B illustrates results of wound closure kinetics for each test sample treated subject. An analysis of FIG. 15B indicates that treatment with PLC and PRP were observed to have faster wound closure kinetics on days 3 and 6 compared to vehicle treatment (*=p<0.05).

FIG. 15C shows histology results of diabetic mouse wounds. Representative histology sections (two representative histology images per condition) of wounds stained with H&E highlight treatment-related differences in wound healing including formation of granulation tissue. Treatment with PLC and PRP both result in an improved granulation tissue area compared to vehicle treatment.

FIGS. 15D-15F provide quantitative analysis of wound closure (FIG. 15D), granulation tissue area (FIG. 15E) and epidermis tissue area (FIG. 15F) based on analysis of would histology images. A significant difference compared to vehicle was observed in granulation tissue area in wounds treated with PLC and PRP (****=p<0.0001). PLC and PRP treatment show trends of increased wound closure and epidermis tissue area.

Histological sections were also treated with a fluorescent endomucin antibody as a proxy for blood vessels within the healing wounds. FIG. 15G shows endomucin-based histological visualization of vasculature for vehicle, PLC (middle) and PRP (right) treated subjects. FIG. 15H shows quantification of the endomucin fluorescent signal, which indicates that PLC and PRP have a statistically significant increase in endomucin compared to vehicle treatment (*=p<0.05).

In sum, the analysis of the results indicates that unique biomarker composition of PLCs advantageously helps in wound healing of diabetic subjects. The wound healing could be associated with increased endomucin concentration in PLCs treated subject.

### Example 12: Making of MLCs

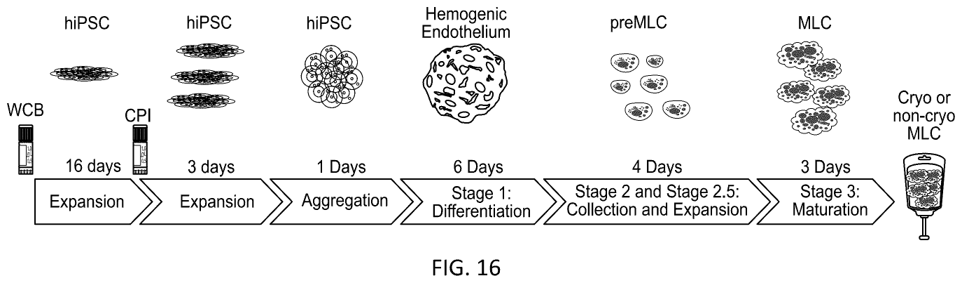

Described herein is a method for making MLCs for use according to any of the embodiments described herein. Briefly, FIG. 16A illustrates a schematic of the differentiation process. Media with specified ingredients were used for each stage to direct cell differentiation into megakaryocyte-like cells (MLC). One vial of the human induced pluripotent stem cell working cell bank (hiPSC WCB) can be expanded to create multiple vials of cryopreserved intermediate (CPI) cells. CPI vials were first thawed to start the differentiation process. After 2D expansion of the hiPSCs from the CPI vials for about 3 days, cells were then harvested and transferred into a vertical rotation device, such as, for example, a vertical wheel bioreactor, to enable 3D aggregation of the hiPSC from a single cell suspension, generally for about 1 day. In Stage 1, aggregates were transferred into spinner flask(s) to start differentiation into hemogenic endothelium for about 6 days. In Stage 2, MLC precursor cells (preMLCs) were released from the aggregated hemogenic endothelia and would be harvested each day into a separate vessel for Stage 2.5 expansion, for about 4 days. Stage 3 was the final maturation process from preMLCs into MLCs, a duration for which was about 3 days. The MLCs were then harvested for cryopreservation and/or megakaryocyte derivative production. Freshly harvested MLCs can be used to optimize processing time and efficiency.

FIG. 16B illustrates further detail regarding Stages 2.5 and 3 (enlarged image of stages 2.5 and 3 differentiation process as shown in FIG. 16A). In particular, FIG. 16B describes the harvest strategy for batch preMLC to MLC maturation culture. Stage 2 to Stage 2.5 is where preMLC optionally released from hemogenic endothelium aggregates. The released cells were collected from the spinner flasks each day and transferred into G-Rex vessels for further expansion and maturation. All G-Rex vessels were harvested as a single batch at the end of the process.

### Example 13: Making of PLCs

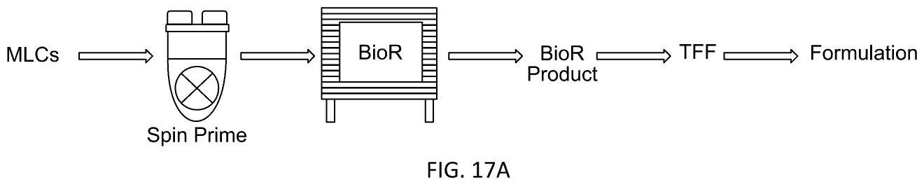

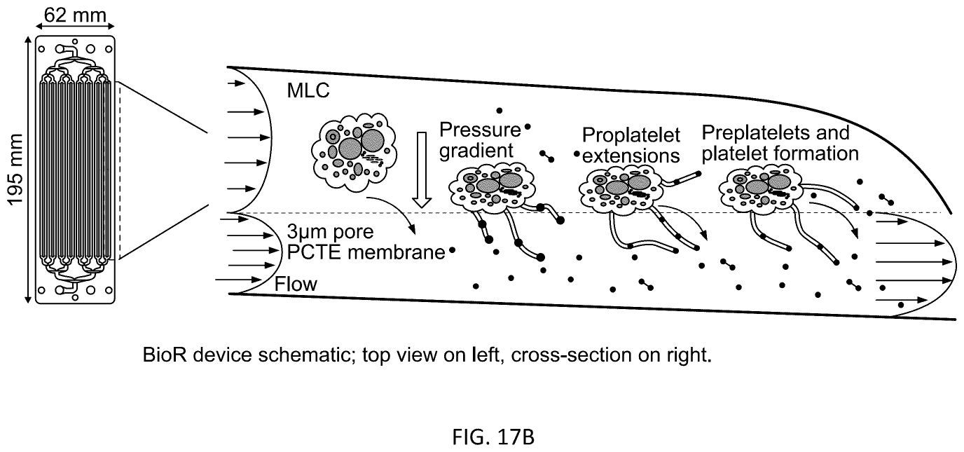

Described herein is a method for making PLCs for use according to any of the embodiments described herein. Briefly, FIG. 17A illustrates a schematic of the PLC production process. MLCs were thawed and centrifuged to remove DMSO and to wash cells. MLCs were then resuspended with BioR media. MLC suspension was spin primed in a ‘PBS’ spinner. MLCs were processed through a biomimetic, microfluidic platelet bioreactor (BioR). BioR product was harvested and processed through TFF˜300-400× concentration and diafiltration with Plasmalyte A. TFF product was analyzed for OD600 and formulated to a target dose of 70 FAU/mL. DP was aliquoted, frozen down and stored at −80° C.

FIG. 17B illustrates an exemplary microfluidic bioreactor (BioR) device for processing the MLCs. The BioR device comprised a 3 μm pore PCTE (polycarbonate track etch) membrane. MLCs were seeded above the membrane, and a pressure gradient was generated to stimulate the MLCs to produce PLCs.

From the foregoing description, it will be apparent that variations and modifications may be made to the embodiments of the present disclosure to adopt it to various usages and conditions. Such embodiments are also within the scope of the following claims. The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1.-44. (canceled)

45. A method of treating a condition comprising:
differentiating an ex-vivo culturing of a population of induced pluripotent stem cells (iPSCs) into mature megakaryocyte-like cells (MLCs);
lysing the mature MLCs to produce lysates;
processing the lysates to form a therapeutic composition, wherein the therapeutic composition comprises one or more of: epidermal growth factor (EGF)>10% higher or interleukin (IL)-10>10% higher as compared to plasma rich in growth factors (PRGF), or EGF>10% higher, IL-10>10% higher as compared to crude lysates; and
administering to a subject suffering from a dry eye disease an effective amount of the therapeutic composition, thereby treating a dry eye disease.

46. The method of claim 45, wherein the lysates further comprise one or more of: EGF>50% higher or IL-10>50% higher as compared to PRGF.

47. The method of claim 45, wherein the lysates further comprise one or more of: matrix metalloproteinase (MMP)-1>10% higher, MMP-9>10% higher, MMP-12, tissue inhibitors of metalloproteinases (TIMP)-1>10% higher, TIMP-3>10% higher, transforming growth factors (TGF)-B1>10% higher, TGF-B2>10% higher, Endoglin>10% higher, Endothelin-3>10% higher, Follistatin>10% higher, heparin-binding (HB)-EGF>10% higher, hepatocyte growth factor (HGF)>10% higher, placental growth factor (PLGF)>10% higher, vascular endothelial growth factor (VEGF)-A>10% higher, soluble CD40L (sCD40L)>10% higher, Eotaxin>10% higher, fibroblast growth factors (FGF)-2>10% higher, FLT-3L>10% higher, Fractalkine>10% higher, granulocyte colony stimulating factor (G-CSF)>10% higher, growth-regulated oncogene a (GROa)>10% higher, interferon (IFN)-a2>10% higher, IFNγ>10% higher, IL-1a>10% higher, IL-1B>10% higher, IL-1RA>10% higher, IL-2>10% higher, IL-3>10% higher, IL-4>10% higher, IL-5>10% higher, IL-6>10% higher, IL-8>10% higher, IL-9>10% higher, IL-10>10% higher, IL-12p40>10% higher, IL-12p70>10% higher, IL-13>10% higher, IL-15>10% higher, IL-17A>10% higher, IL-17E>10% higher, IL-25>10% higher, IL-17F>10% higher, IL-22>10% higher, IL-27>10% higher, interferon gamma-induced protein (IP)-10>10% higher, monocyte chemoattractant protein (MCP)-1>10% higher, MCP-3>10% higher, macrophage-derived chemokine (MDC)>10% higher, macrophage inflammatory protein (MIP)-1a>10% higher, MIP-1b>10% higher, platelet-derived growth factor (PDGF)-AA>10% higher, transforming growth factor (TGF) a>10% higher, tumor necrosis factor (TNF) a>10% higher, TNFB>10% higher, a proliferation-inducing ligand (APRIL)>10% higher, B-cell activating factor (BAFF)>10% higher, regulated upon activation, normal T cell expressed and secreted (RANTES)>10% lower, IL-7>10% lower, VEGF-D>10% lower, VEGF-C>10% lower, Leptin>10% lower, angiopoietin (Ang) 2>10% lower, transforming growth factor (TGF) B-3>10% lower, MMP-10>10% lower, MMP-7>10% lower, MMP-3>10% lower, or MMP-2>10% lower as compared to PRGF as compared to PRGF.

48. The method of claim 45, wherein the lysates further comprise one or more of: MMP-7>10% higher, TIMP-1>10% higher, TIMP-3>10% higher, IL-2>10% higher, IL-15>10% higher, IL-17F>10% higher, granulocyte colony stimulating factor (G-CSF)>10% higher, MMP-13>10% lower, TGFB-1>10% lower, IL-5>10% lower, APRIL>10% lower, or BAFF>10% lower as compared to crude lysates.

49. The method of claim 45, wherein the method improves one or more signs and/or symptoms of dry eye disease.

50. The method of claim 45, wherein the dry eye disease is caused by Sjogren's syndrome or non-Sjogren's syndrome.

51. The method of claim 45, wherein the therapeutic composition is diluted to a physiological concentration in a carrier, wherein the carrier comprises a diluent or an excipient.

52. The method of claim 45, wherein the therapeutic composition further comprises a wound healing agent, a tissue regeneration agent, an antiapoptotic agent, an anti-inflammatory agent, a neurotropic agent, an anti-hormonal agent or an immunomodulatory agent or a combination thereof.

53. The method of claim 52, wherein the tissue regeneration agent is one or more of (a) a growth factor selected from one or more of transforming growth factors (TGF), fibroblast growth factors (FGF), platelet-derived growth factors (PDGF), epidermal growth factors (EGF), vascular endothelial growth factors (VEGF), insulin-like growth factors (IGF), platelet-derived endothelial growth factors (PDEGF), platelet-derived angiogenesis factors (PDAF), platelet factors 4 (PF-4), hepatocyte growth factors (HGF) or combinations thereof; and (b) a cytokine selected from one or more of IL-1B, IL-5, IL-6, IL-8, IL-10, IL-12, IL-13, IL-17A, IL-23, TNF alpha or combinations thereof.

54. The method of claim 45, wherein the population of cells comprises cells derived from immortalization, reprogramming or differentiation of a somatic cell, a progenitor cell or a stem cell.

55. The method of claim 45, wherein the therapeutic composition further comprises lysates, serum, plasma, plasma rich in growth factors (PRGF), platelet rich plasma (PRP), or any other blood derivative derived from the subject.

56. The method of claim 45, wherein the therapeutic composition in formulated in a buffer, diluent, or excipient or a combination thereof.

57. The method of claim 45, wherein the therapeutic composition comprises: a) between 0.01 and 100 wt % of the lysates, b) between 0 and 90 wt. % of a bulking agent, and/or c) between 0 and 90 wt. % of at least one excipient or carrier and optionally d) platelet-rich plasma (PRP), PRGF, plasma, serum, or other blood derivative derived from the subject.

58. The method of claim 45, wherein the therapeutic composition is lyophilized.

59. The method of claim 45, wherein the administering comprises locally administering the therapeutic composition at one or more of a site of or near an injury or a disease,
wherein the therapeutic composition is administered by any one of route of administrations selected from topical, transdermal, or systemic route of administration, or
wherein the therapeutic composition is administered by any one of route of administrations selected from subconjunctival, sub-Tenon's, intravitreal, intracameral, intravenous, intraarterial, intramuscular, subcutaneous, inhalation, rectal, buccal, vaginal, intraperitoneal, intraarticular, intraglandular into a lacrimal gland, topical, otic, or oral route of administration.

60. A method of treating a condition comprising: administering to a subject suffering from a dry eye disease an effective amount of a therapeutic composition, the therapeutic composition comprising lysates of megakaryocyte-like cells (MLCs), wherein, after processing, the lysates comprise one or more of: epidermal growth factor (EGF)>10% higher or interleukin (IL)-10>10% higher as compared to plasma rich in growth factors (PRGF), or EGF>10% higher, IL-10>10% higher as compared to crude lysates.

61. A method for producing a therapeutic composition comprising an effective amount of processed lysates of MLCs, the method comprising:
an ex-vivo culturing of a population of iPSCs for a duration to differentiate the iPSCs to mature MLCs;
concentrating the MLCs;
lysing the MLCs; and
processing the lysed MLCs to form the therapeutic composition;
wherein, after processing, the therapeutic composition comprises one or more of: epidermal growth factor (EGF)>10% higher or interleukin (IL)-10>10% higher as compared to plasma rich in growth factors (PRGF), or EGF>10% higher, IL-10>10% higher as compared to crude lysates.

62. The method of claim 61, wherein the processing comprises filtering the lysed MLCs.

63. The method of claim 61, wherein the processing comprises centrifuging the lysed MLCs.

64. A therapeutic composition for treating a dry eye disease in a subject, the therapeutic composition comprising an effective amount of an MLC lysate, wherein the MLC lysate comprise one or more of: epidermal growth factor (EGF)>50% higher or interleukin (IL)-10>50% higher as compared to plasma rich in growth factors (PRGF), or EGF>50% higher, IL-10>50% higher as compared to crude lysates.

65. The therapeutic composition of claim 64, wherein the MLC lysate further comprises one or more of: fibroblast growth factor-2 (FGF-2); hepatocyte growth factor (HGF); insulin-like growth factor 1 (IGF-1), regulated upon activation, normal T cell expressed and secreted (RANTES); nerve growth factor (NGF); vascular endothelial growth factor (VEGF-A); vascular endothelial growth factor (VEGF-C); epidermal growth factor (EGF); transforming growth factor-β1 (TGF-β1); transforming growth factor-β2 (TGF-β1); platelet-derived growth factor-AA (PDGF-AA); platelet-derived growth factor-BB (PDGF-BB); platelet-derived growth factor-AA/BB (PDGF-AA/BB); interleukin 2 (IL-2); interleukin 4 (IL-4); interleukin 12p40 (IL-12p40); interleukin 12p70 (IL-12p70); tissue inhibitors of metalloproteinases-1 (TIMP-1); tissue inhibitors of metalloproteinases-2 (TIMP-2); or tissue inhibitors of metalloproteinases-3 (TIMP-3).

66. The therapeutic composition of claim 64, wherein the therapeutic composition further comprises another therapeutic agent.
